(19) United States
(12) Patent Application Publication
(10) Pub. No.: US 2015/0216993 A1
(43) Pub. Date: Aug. 6, 2015

(54) MULTIFUNCTIONAL SMALL MOLECULES

(71) Applicant: The Regents of the University of Michigan, Ann Arbor, MI (US)

(72) Inventors: James R. Baker, JR., Ann Arbor, MI (US); Seok Ki Choi, Ann Arbor, MI (US); Abraham F. L. Van Der Spek, Ann Arbor, MI (US); Pascale Leroueil, Ann Arbor, MI (US)

(21) Appl. No.: 13/862,951

(22) Filed: Apr. 15, 2013

Related U.S. Application Data
(60) Provisional application No. 61/625,911, filed on Apr. 18, 2012.

(51) Int. Cl. A61K 47/48; C12N 9/18; A61K 45/06; C08G 73/02; A61K 31/4425; A61K 31/16

(52) U.S. Cl. CPC A61K 47/48207; A61K 31/4425; A61K 31/16; A61K 47/48215; A61K 45/06; C08G 73/028; C12N 9/18; C12Y 301/01007

(57) ABSTRACT

The present invention relates to novel therapeutic dendrimer conjugates configured for the treatment and/or prevention of organophosphate poisoning. In particular, the present invention is directed to dendrimers complexed with organophosphate poisoning antidotes (e.g., pralidoxime (2-PAM) (4-PAM), obidoxime, trimedoxime, asoxime (HI-6), hydroxamate, and related analogs, salts and derivatives thereof), compositions comprising such dendrimer conjugates, related methods of synthesizing such dendrimer conjugates, as well as systems and methods utilizing such dendrimer conjugates (e.g., in diagnostic and/or therapeutic settings (e.g., for the delivery of therapeutics, imaging, and/or targeting agents (e.g., in the treatment and/or prevention of organophosphate poisoning)).

### STATEMENT REGARDING FEDERALLY SPONSORED RESEARCH OR DEVELOPMENT

This invention was made with government support under Grant No. W911NF-07-1-0437 awarded by the U.S. Army Research Office. The government has certain rights in the invention.

### FIELD OF THE INVENTION

The present invention relates to novel therapeutic dendrimer conjugates configured for the treatment and/or prevention of organophosphate poisoning. In particular, the present invention is directed to dendrimers complexed with organophosphate poisoning antidotes (e.g., pralidoxime (2-PAM) (4-PAM), obidoxime, trimedoxime, asoxime (HI-6), hydroxamate, and related analogs, salts and derivatives thereof), compositions comprising such dendrimer conjugates, related methods of synthesizing such dendrimer conjugates, as well as systems and methods utilizing such dendrimer conjugates (e.g., in diagnostic and/or therapeutic settings (e.g., for the delivery of therapeutics, imaging, and/or targeting agents (e.g., in the treatment and/or prevention of organophosphate poisoning)).

### BACKGROUND OF THE INVENTION

Reactive organophosphates are a class of phosphate-based neurotoxic agents. These compounds cause life threatening symptoms by inhibiting acetylcholine esterase (AChE) and pose serious threats to both the armed forces and civilian population. Examples of organophosphates include weaponized nerve agents such as Sarin and VX as well as a number of insecticides commonly used in the agriculture industry. Clinically approved antidotes for organophosphate poisoning include pralidoxime and obidoxime. Unfortunately, these therapeutics are only effective for a short window of time (about 10 minutes) due to their rapid clearance from the body. As such, improved techniques for treating organophosphate poisoning are needed.

### SUMMARY OF THE INVENTION

Experiments conducted during the course of developing embodiments for the present invention developed a multifunctional nanoscale particle derived from a dendrimer (e.g., polyamidoamine (PAMAM), Baker-Huang dendrimer), wherein the dendrimer is complexed with an OP antidote (e.g., pralidoxime (2-PAM) (4-PAM), obidoxime, trimedoxime, asoxime (HI-6), hydroxamate, and related analogs, salts and derivatives thereof) (see, e.g., FIG. 17). Such experiments demonstrated that the nanoparticle is rationally designed to have three distinct functions. First, the nanoparticle serves as a drug carrier by providing drug-binding cavities for OP antidotes (e.g., 2-PAM molecules) and enables to extend the duration of drug action through a sustained release mechanism. Second, the nanoparticle itself displays built-in therapeutic activity as an OP scavenger and the AChE reactivator (see, e.g., FIG. 2). Third, drug release within the nanopaticle is triggered by a feedback-regulated mechanism where the dendrimer drug carrier releases the OP antidote (e.g., 2-PAM) payloads in response to its OP scavenging action. Indeed, it was determined that the sustained release of the OP antidote in combination with the feedback release mechanism is a substantial improvement for the treatment of acute exposures to neurotoxic agents. In addition, such nanoparticles represent a suitable prophylactic option against OP poisoning.

Accordingly, the present invention provides novel therapeutic dendrimer conjugates configured for the treatment and/or prevention of organophosphate poisoning. In particular, the present invention provides dendrimers complexed with organophosphate poisoning antidotes (e.g., pralidoxime (2-PAM) (4-PAM), obidoxime, trimedoxime, asoxime (HI-6), hydroxamate, and related analogs, salts and derivatives thereof), compositions comprising such dendrimer conjugates, related methods of synthesizing such dendrimer conjugates, as well as systems and methods utilizing such dendrimer conjugates (e.g., in diagnostic and/or therapeutic settings (e.g., for the delivery of therapeutics, imaging, and/or targeting agents (e.g., in the treatment and/or prevention of organophosphate poisoning)).

In certain embodiments, the present invention provides methods for treating a subject having organophosphate poisoning. In certain embodiments, the present invention provides methods for preventing a subject from developing organophosphate poisoning. In some embodiments, the methods involve, for example, administering to the subject an effective amount of one or more dendrimers conjugated with one or more therapeutic agents, wherein the one or more therapeutic agents comprises one or more organophosphate poisoning antidote agents.

The methods are not limited to particular dendrimer molecules. In some embodiments, the dendrimer is a classical PAMAM dendrimer. In some embodiments, the dendrimer is a Baker-Huang dendrimer. In some embodiments, the dendrimer has a generation between 0 and 5.

The methods are not limited to a particular organophosphate. For example, in some embodiments the organophosphate is parathion, paraoxon, sarin, and/or VX.

The methods are not limited to particular organophosphate antidotes. For example, in some embodiments, the organophosphate antidote is pralidoxime (2-PAM) (4-PAM), obidoxime, trimedoxime, hydroxamate, and/or asoxime (HI-6).

The methods are not limited to a particular manner of conjugation between the dendrimer and the organophosphate poisoning antidote. In some embodiments, the organophosphate poisoning antidote covalently binds directly with the dendrimer. In some embodiments, the organophosphate poisoning antidote is conjugated with the dendrimer via a spacing agent. In some embodiments, the spacing agent comprises a oligoethyleneglycol linear chain.

The methods are not limited to a particular manner or treating and/or preventing organophosphate poisoning. In some embodiments, administration of the dendrimer to the subject results in hydrolysis of organophosphate molecules. In some embodiments, administration of the dendrimer to the subject results in reactivation of inhibited acetylcholine esterase.

In some embodiments, the dendrimer is co-administered with one or more additional agents known to be effective in treating organophosphate poisoning, wherein the additional agents are selected from the group consisting of oxime agents, anticholinergic agents, and benzodiazepine agents.

In certain embodiments, the present invention provides compositions comprising one or more dendrimers conjugated with one or more therapeutic agents, wherein the one or more therapeutic agents comprises one or more organophosphate poisoning antidote agents.

In some embodiments, the dendrimer is a classical PAMAM dendrimer. In some embodiments, the dendrimer is a Baker-Huang dendrimer. In some embodiments, the dendrimer has a generation between 0 and 5.

The methods are not limited to particular organophosphate antidotes. For example, in some embodiments, the organophosphate antidote is pralidoxime (2-PAM) (4-PAM), obidoxime, trimedoxime, hydroxamate, and/or asoxime (HI-6).

The methods are not limited to a particular manner of conjugation between the dendrimer and the organophosphate poisoning antidote. In some embodiments, the organophosphate poisoning antidote covalently binds directly with the dendrimer. In some embodiments, the organophosphate poisoning antidote is conjugated with the dendrimer via a spacing agent. In some embodiments, the spacing agent comprises a oligoethyleneglycol linear chain.

In certain embodiments, the present invention provides a dendrimer conjugate comprising both oxime-based therapeutic molecules and auxiliary groups such as metal chelators (FIG. 56). The therapeutic benefit for attaching such auxiliary groups is illustrated in the proposed mechanism of OP (PDX) hydrolysis where the auxiliary group plays a significant role by facilitating the catalytic reaction mediated by the oxime or hydroxamate of the attached drug molecule. Examples of those metal chelating auxiliary groups are based, for example, on the amine, imidazole, pyridine, and carboxylate groups, and include Tren, PDA, and PCA, but not limited here. Metal ions to be chelated include, but are not limited to, zinc, copper and other physiologic cations that are able to chelate to the P═O of the OP molecule and to make the phosphorous bond more susceptible for the hydrolytic cleavage.

Additional embodiments will be apparent to persons skilled in the relevant art based on the teachings contained herein.

### BRIEF DESCRIPTION OF THE DRAWINGS

FIG. 1 shows a complex formation between a generation 5 (G5) PAMAM dendrimer and 2-PAM.

FIG. 2 displays built-in therapeutic activity as an OP scavenger and the AChE reactivator for the dendrimer conjugates of the present invention.

FIG. 3 shows the structure of one embodiment of a generation 1 (G1) Baker-Huang NH₂-terminated PAMAM dendrimer as compared to the structure of a generation 1 (G1) Tomalia NH₂-terminated PAMAM dendrimer. Notably, the central region of the Baker-Huang dendrimer embodiment includes additional NH— and ═O groups, which provide potential points of attachment for functional ligands (e.g., targeting agents, therapeutic agents, imaging agents, trigger agents).

FIG. 4 shows an embodiment of an AB₂ branch unit of the present invention.

FIG. 5 shows a diagram of a dendrimer conjugate provided in some embodiments of the present invention.

FIG. 6 shows a diagram of a dendrimer conjugate provided in some embodiments of the present invention.

FIG. 7 shows a diagram of a dendrimer conjugate provided in some embodiments of the present invention.

FIG. 8 shows a diagram of a dendrimer conjugate provided in some embodiments of the present invention.

FIG. 9 shows a diagram of a dendrimer conjugate provided in some embodiments of the present invention.

FIG. 10 shows the release of a therapeutic compound from a dendrimer conjugate in one embodiment of the invention.

FIG. 11 shows the release of a therapeutic compound from a dendrimer conjugate in one embodiment of the invention.

FIG. 12 depicts a dendrimer conjugate comprising a cyclization based linker in some embodiments of the present invention.

FIG. 13 depicts cyclization based linkers in some embodiments of the invention.

FIGS. 14A and B depicts dendrimer conjugates provided in some embodiments of the present invention.

FIG. 15 shows a dendrimer comprising a simple ester (top portion of figure) and a dendrimer conjugate comprising an elimination linker (e.g., a 1, 6, elimination linker/spacer as shown in the bottom portion).

FIG. 16 shows structures for the reactivators of acetylcholine esterase (AChE): pralidoxime, obidoxime, trimedoxime, and HI-6.

FIG. 17 shows representative examples of dendrimer drug conjugates, each covalently linked with hydroxamate, 2-PAM or 4-PAM molecules.

FIG. 18A shows a general synthesis scheme for G5-glutaryl hydroxamate (G5-GHA).

FIG. 18B shows synthetic scheme for the G5 dendrimers conjugated with glutaric hydroxamate (G5-GHA; n=number of hydroxamate=19, 66).

FIG. 19 shows HPLC traces for three related G5 dendrimers, unmodified G5-NH₂, G5-GA, and G5-GHA (n=66); each analyzed at 1 mg/mL.

FIG. 20 shows comparison of ¹H NMR spectra of G5-GA and G5-GHA (n=number of hydroxamate=66), each acquired in D₂O.

FIG. 21 shows MALDI TOF spectra for G5-GHA (n=19), and G5-GA.

FIG. 22 shows synthetic scheme for G5-GHAcp, the G5 dendrimer conjugated with cyclopentane-fused glutaryl hydroxamate.

FIG. 23 shows HPLC traces for G5-GAcp, and G5-GHAcp; each analyzed at 1 mg/mL.

FIG. 24 shows synthesis of 4-PAM linker-NH₂.

FIG. 25 shows synthesis of 2-PAM linker-NH₂.

FIG. 26 shows synthesis of PAM linker-CO₂H.

FIG. 27 shows synthesis of G5-(2PAM)ₙ (n=13).

FIG. 28 shows characterization of G5-(2PAM)ₙ (n=13) by UV/vis spectrometry (A), HPLC (B), and MALDI TOF mass spectrometry (C).

FIG. 29 shows synthesis of G5-(4PAM)ₙ (n=19).

FIG. 30 shows characterization of G5-(4PAM)ₙ (n=19) by UV/vis spectrometry (A), HPLC traces (B), ¹H NMR (D₂O) (C), and MALDI TOF mass spectrometry (D).

FIG. 31 shows synthesis of G5-EG-(Hydroxamate)-(4PAM).

FIG. 32 shows characterization of G5-EG as compared to G5-GA: GPC traces (A), HPLC traces (B), and ¹H NMR spectroscopy (D₂O; C)

FIG. 33 shows HPLC characterization of G5-EG-(Hydroxamate).

FIG. 34 shows characterization of G5-EG-(4PAM) by HPLC (A), and ¹H NMR spectroscopy (D₂O; B).

FIG. 35 shows characterization of G5-EG-(Hydroxamate)-(4PAM) by HPLC (A), and UV/vis spectrometry (B).

FIG. 36A shows observed rate constant (k_{obsd}) for the paraoxon (PDX; 0.01 mM) hydrolysis catalyzed by 2-PAM (0.5 mM), obidoxime (0.5 mM), and two dendrimer hydroxamates (G5-GHA, 0.028 mM), each having different number of hydroxamate branches (n=16; n=66). The PDX hydrolysis was evaluated at rt and at two different pH conditions (pH=7.4, and 9.0).

FIG. 36B displays mass spectrometric evidence for the formation of oxime-paraoxon adduct.

FIG. 36C shows the rate constant (k₁) for PDX hydrolysis catalyzed by obidoxime.

FIG. 37 shows observed rate constant (k_{obsd}) for the PDX (0.03 mM) hydrolysis catalyzed by PAM linker molecules, each tested at 1.5 mM at rt and in PBS (pH 7.4).

FIG. 38 shows ¹H NMR spectroscopy for monitoring the kinetics of paraoxon (PDX; 0.5 mM) hydrolysis catalyzed by 2-PAM (0.5 mM) in deuterated PBS (pH 7.4). The PDX hydrolysis was performed at rt as a function of time, and the ¹H NMR spectra taken at t=0 hr (A), 12 hr (B), 191 hr (C) are shown. Those reference spectra are shown further below that include 2-PAM alone and PDX alone, each taken at the identical condition.

FIG. 39 shows summary for the PDX scavenging activity of 2-PAM and G5-GHA (n=19; χ=0.17) as determined by ¹H NMR spectroscopy. In this illustrative study, paraoxon (PDX; 4.5 mM) was dissolved in deuterated PBS (pH 7.4), and 2-PAM or each dendrimer conjugate was tested at the concentration as indicated in the plot.

FIG. 40A shows rates of PDX (0.48 mM) hydrolysis catalyzed by G5-GHA (0.049 mM) alone or G5-GHA/2-PAM complexes.

FIG. 40B shows ¹H NMR spectroscopy for determining the kinetics of paraoxon (PDX; 0.5 mM) hydrolysis catalyzed by G5-GHA (n=66; 0.05 mM) in deuterated PBS (pH 7.4). The PDX hydrolysis was studied at rt. Note that 4-NP (4-nitrophenol), and PA (diethylphosphoric acid) are the two degradation products of PDX.

FIG. 41 shows ¹H NMR titration experiments for a G5 PAMAM dendrimer and pralidoxime (2-PAM) in D₂O. ¹H NMR spectral regions for the unmodified, amine-terminated dendrimer (A) and 2-PAM (B). G5 dendrimer alone ([D]=6.23×10⁻⁴ M) (i) and dendrimer-drug complexes prepared at [2-PAM]/[D]=1 (ii), 10 (iii), 21 (iv), 42 (v), 63 (vi), 84 (vii), 125 (viii), and 2-PAM alone (ix). (C) Changes in chemical shift values (e.g., Δ=(δ_{c,viii}−δ_{c,i}), ppm) for dendrimer protons plotted against [2-PAM]/[D] ratio.

FIG. 42 shows models proposed for complexation of oxime drugs to G5 PAMAM dendrimer. The mean number (114) of terminal amines per dendrimer is determined by potentiometric titration where the molar amount of the dendrimer sample is calculated on the basis of its MALDI molecular weight (27 600 g mol⁻¹).

FIG. 43 shows 1H NMR spectral signals for pralidoxime chloride acquired in D2O, the drug alone (60 mM, A), in the same solvent containing 1:1 molar equivalents of triethylamine (B), or ethanolamine (C), and in complex with G5 PAMAM dendrimer (6.23×10-4 M) (D) where the molar ratio of pralidoxime to the dendrimer is 10. Note: Figure (C) is 1H NMR spectrum of an equimolar mixture comprised of 2-PAM and ethanolamine. The signals at ˜3.8 and ˜3.0 ppm are all from enthanolamine. In Figure (B), those signals at ˜3.1 and ˜1.3 ppm are from Et3N, and in Figure (D), the dendrimer signals appear at 3.4-2.3 ppm.

FIG. 44 shows 1H NMR spectra obtained from titration experiments. (A) G5 PAMAM/pralidoxime (2-PAM) in deuterated PBS, pH 7.4; (B) G5 PAMAM/pralidoxime in D2O; (C) G5 PAMAM/Obidoxime in D2O; (D) G5 PAMAM/N-methylpyridinium chloride (MPC) in D2O. The concentration of the PAMAM dendrimer used for each experiment is 6.23×10-4M except for (B) where it is 6.04×10-5M.

FIG. 45 shows change in the chemical shift values (Δ, ppm) for the protons of G5 PAMAM dendrimer upon complexation with pralidoxime (2-PAM) in PBS pH 7.4 (A), and with obidoxime in D2O (B). The Δ value for each set of the protons refers to the difference in the chemical shift values observed before and after the complexation with the oxime drug. For example, the value for the e signal for a complex ([2-PAM]/[D]=85) is equal to the difference, ( ) e,85 e,0 δ−δ. It is plotted as the function of the molar ratio of the drug to the dendrimer. The concentration of the PAMAM dendrimer used for each experiment is 6.23×10-4M.

FIG. 46 shows two dimensional (2D) proton NMR spectra for G5 PAMAM dendrimer in complex with pralidoxime in PBS, pH 7.4 ([D]=6.23×10-4M; [2-PAM]/[D]=146). (A) 1H-1H COSY spectrum: cross peaks in the dashed rectangles indicate the scalar coupling between the marked protons from either pralidoxime or the dendrimer. (B) NOESY spectrum: cross peaks shown in the dashed rectangles indicate the through-space intramolecular correlation, and those in the dashed circle indicate the intermolecular correlation between the pralidoxime proton (H1) and the dendrimer protons (e, c, d).

FIG. 47 shows two dimensional proton NMR spectra for G5 PAMAM dendrimer in complex with obidoxime in D2O ([D]=6.23×10-4M; [Obidoxime]/[D]=80). (A) 1H-1H COSY spectrum. (B) 1H-1H NOESY spectrum.

FIG. 48 shows (A) A representative pseudo-2D DOSY plot for G5 PAMAM dendrimer in complex with 2-PAM ([D]=6.04×10⁻⁵ M; [2-PAM]/[D]=123.5). (B, C) Diffusion coefficients (D, m² s⁻¹), and hydrodynamic radii (Rₕ, nm) for G5/2-PAM complexes, each plotted as a function of [2-PAM]/[D] ratio. Diffusion coefficient determined for each complex in (B) refers to a mean value obtained from at least two independent sets of measurements, and the error represents the standard deviation from the mean value.

FIG. 49 shows representative spectra from Diffusion Ordered Spectroscopy (DOSY) experiments for G5 PAMAM dendrimer, and its complexes with pralidoxime (2-PAM) prepared at the variable molar ratio of the oxime to dendrimer. Note that the operation principle of DOSY experiments is to acquire a series of 1D 1H NMR spectra as a function of G (gradient field strength; G0 equals to the parameter set to acquire a standard 1H NMR spectrum). Each series of 1H NMR spectra shown in Figure (A) to (D) comprises of the typical 1H NMR spectrum at G0, and followed by those spectra recorded as G is varied. This systematic G variation leads to the decrease of the peak intensity. The bottom spectrum (G0) in Figure (A) is the typical 1H NMR spectrum taken for G5 PAMAM dendrimer in D2O. The bottom spectrum in (B) is the 1H NMR spectrum for dendrimer-PAM complex ([PAM]/[D]=41). Thus, there are changes in chemical shifts in both PAM and dendrimer region in accordance with the dendrimer-PAM complexation. This principle applies equally to those DOSY spectra in FIGS. (C) and (D).

FIG. 50 shows quantitative analysis for the complexation of G5 dendrimer with two oxime drugs. (A) Number of bound molecules and (B) fraction of occupied binding sites (θ), plotted against the ratio [oxime]/[D]. (C) Scatchard plots for dendrimer-drug complexation in D₂O. (D) Steady-state dissociation constants (K_{D}) plotted as a function of θ.

FIG. 51 shows Hill plots for the complexation of G5 PAMAM dendrimer with pralidoxime, and obidoxime in D2O. The value of Hill coefficient (n) for the dendrimer complexation is 0.58 (pralidoxime), and 0.49 (obidoxime). It was determined from the slope of the plot according to the Hill equation: log(θ/1−θ)=nlog[Oxime]−log Kd. The parameter θ refers to the fraction of occupied binding sites.

FIG. 52 shows the UV/vis spectrometry for 4-Nitrophenol production from 2-PAM and PDX.

FIGS. 53A and B show additional data related to hydrolysis of paraoxon catalyzed by 2-PAM.

FIG. 54 shows that 2-PAM derivatives are catalytically active for PDX hydrolysis.

FIG. 55 shows kinetics of PDX hydrolysis.

FIG. 56 shows (A) Structures of the G5-(oxime antidote) conjugates, each conjugated with auxiliary metal chelating groups in addition to the pyridiniumaldoxime or hydroxamate; (B) and (C) The proposed mechanism of OP (PDX) hydrolysis that illustrates the assistant role played by the auxiliary group such as Tren-Zn or PDA-Zn.

FIG. 57 shows representative synthesis of G5-PAM-(X) or G5-GHA-(X) where X refers to an auxiliary Zn chelator.

FIG. 58 summarizes the hydrolysis (%) of paraoxon catalyzed by 2-PAM or G5-(GHA)ₙ₌₆₆, each tested in guinea pig plasma.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having organophosphate poisoning” refers to a subject that presents one or more symptoms indicative of organophosphate poisoning. Symtpoms indicative of organophosphate poisoning include, but are not limited to, excessive acetylcholine (ACh) present at different nerves and receptors in the body, overstimulation of nicotinic expression at the neuromuscular junction, muscle weakness, fatigue, muscle cramps, fasciculation, paralysis, tachycardia, hypertension, hypoglycemia, anxiety, headache, convulsions, ataxia, depression of respiration and circulation, tremor, general weakness, visual disturbances, tightness in chest, wheezing due to bronchoconstriction, increased bronchial secretions, increased salivation, lacrimation, sweating, and/or peristalsis (see, e.g., Leibson T, Lifshitz M (2008) J Toxicology 10: 767-7704; Eskenazi B, Bradman A, Castorina R (1999) J Environmental Health Perspectives 107: 409-419; each herein incorporated by reference in its entirety).

As used herein, the term, “subject at risk for organophosphate poisoning” refers to a subject that is at risk for exposure to any type of organophosphate.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “drug” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “purified” or “to purify” or “compositional purity” refers to the removal of components (e.g., contaminants) from a sample or the level of components (e.g., contaminants) within a sample. For example, unreacted moieties, degradation products, excess reactants, or byproducts are removed from a sample following a synthesis reaction or preparative method.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using screening methods known in the art.

As used herein, the term “nanodevice” or “nanodevices” refer, generally, to compositions comprising dendrimers of the present invention. As such, a nanodevice may refer to a composition comprising a dendrimer of the present invention that may contain one or more ligands, linkers, and/or functional groups (e.g., a therapeutic agent, a targeting agent, a trigger agent, an imaging agent) conjugated to the dendrimer.

As used herein, the term “degradable linkage,” when used in reference to a polymer refers to a conjugate that comprises a physiologically cleavable linkage (e.g., a linkage that can be hydrolyzed (e.g., in vivo) or otherwise reversed (e.g., via enzymatic cleavage). Such physiologically cleavable linkages include, but are not limited to, ester, carbonate ester, carbamate, sulfate, phosphate, acyloxyalkyl ether, acetal, and ketal linkages (See, e.g., U.S. Pat. No. 6,838,076; herein incorporated by reference in its entirety). Similarly, the conjugate may comprise a cleavable linkage present in the linkage between the dendrimer and functional group, or, may comprise a cleavable linkage present in the polymer itself (See, e.g., U.S. Pat. App. Nos. 20050158273 and 20050181449, each of which is herein incorporated by reference in its entirety).

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond (e.g., typically a covalent bond) that is substantially stable in water (i.e., does not undergo hydrolysis under physiological conditions to any appreciable extent over an extended period of time). Examples of hydrolytically stable linkages include, but are not limited to, carbon-carbon bonds (e.g., in aliphatic chains), ethers, amides, urethanes, and the like.

As used herein, the term “NAALADase inhibitor” refers to any one of a multitude of inhibitors for the neuropeptidase NAALADase (N-acetylated-alpha linked acidic dipeptidase). Such inhibitors of NAALADase have been well characterizied. For example, an inhibitor can be selected from the group comprising, but not limited to, those found in U.S. Pat. No. 6,011,021, herein incorporated by reference in its entirety.

As used herein, the term “Baker-Huang dendrimer” or “Baker-Huang PAMAM dendrimer” refers to a dendrimer comprised of branching units of structure:

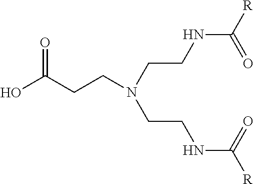

wherein R comprises a carbon-containing functional group (e.g., CF₃). In some embodiments, the branching unit is activated to its FINS ester. In some embodiments, such activation is achieved using TSTU. In some embodiments, EDA is added. In some embodiments, the dendrimer is further treated to replace, e.g., CF₃ functional groups with NH₂ functional groups; for example, in some embodiments, a CF₃-containing version of the dendrimer is treated with K₂CO₃ to yield a dendrimer with terminal NH₂ groups (for example, as shown in Scheme 2). In some embodiments, terminal groups of a Baker-Huang dendrimer are further derivatized and/or further conjugated with other moieties. For example, one or more functional ligands (e.g., for therapeutic, targeting, imaging, or drug delivery function(s)) may be conjugated to a Baker-Huang dendrimer, either via direct conjugation to terminal branches or indirectly (e.g., through linkers, through other functional groups (e.g., through an OH— functional group)). In some embodiments, the order of iterative repeats from core to surface is amide bonds first, followed by tertiary amines, with ethylene groups intervening between the amide bond and tertiary amines. In preferred embodiments, a Baker-Huang dendrimer is synthesized by convergent synthesis methods.

As used herein, the term “scaffold” refers to a compound to which other moieties are attached (e.g., conjugated). In some embodiments, a scaffold is conjugated to bioactive functional conjugates (e.g., a therapeutic agent, a targeting agent, a trigger agent, an imaging agent). In some embodiments, a scaffold is conjugated to a dendrimer (e.g., a PAMAM dendrimer). In some embodiments, conjugation of a scaffold to a dendrimer and/or a functional conjugate(s) is direct, while in other embodiments conjugation of a scaffold to a dendrimer and/or a functional conjugate(s) is indirect, e.g., an intervening linker is present between the scaffold compound and the dendrimer, and/or the scaffold and the functional conjugate(s).

As used herein, the term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, conjugation between a dendrimer (e.g., a terminal arm of a dendrimer) and a functional ligand is accomplished during a “one-pot” reaction. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino) pyridine) (see, e.g., International Patent Application No. PCT/US2010/042556, herein incorporated by reference in its entirety).

As used herein, the term “solvent” refers to a medium in which a reaction is conducted. Solvents may be liquid but are not limited to liquid form. Solvent categories include but are not limited to nonpolar, polar, protic, and aprotic.

As used herein, the term “dialysis” refers to a purification method in which the solution surrounding a substance is exchanged over time with another solution. Dialysis is generally performed in liquid phase by placing a sample in a chamber, tubing, or other device with a selectively permeable membrane. In some embodiments, the selectively permeable membrane is cellulose membrane. In some embodiments, dialysis is performed for the purpose of buffer exchange. In some embodiments, dialysis may achieve concentration of the original sample volume. In some embodiments, dialysis may achieve dilution of the original sample volume.

As used herein, the term “precipitation” refers to purification of a substance by causing it to take solid form, usually within a liquid context. Precipitation may then allow collection of the purified substance by physical handling, e.g. centrifugation or filtration.

As used herein, an “ester coupling agent” refers to a reagent that can facilitate the formation of an ester bond between two reactants. The present invention is not limited to any particular coupling agent or agents. Examples of coupling agents include but are not limited to 2-chloro-1-methylpyridium iodide and 4-(dimethylamino) pyridine, or dicyclohexylcarbodiimide and 4-(dimethylamino) pyridine or diethyl azodicarboxylate and triphenylphosphine or other carbodiimide coupling agent and 4-(dimethylamino)pyridine.

As used herein, the term “glycidolate” refers to the addition of a 2,3-dihydroxylpropyl group to a reagent using glycidol as a reactant. In some embodiments, the reagent to which the 2,3-dihydroxylpropyl groups are added is a dendrimer. In some embodiments, the dendrimer is a PAMAM dendrimer. Glycidolation may be used generally to add terminal hydroxyl functional groups to a reagent.

As used herein, the term “ligand” refers to any moiety covalently attached (e.g., conjugated) to a dendrimer branch; in preferred embodiments, such conjugation is indirect (e.g., an intervening moiety exists between the dendrimer branch and the ligand) rather than direct (e.g., no intervening moiety exists between the dendrimer branch and the ligand). Indirect attachment of a ligand to a dendrimer may exist where a scaffold compound intervenes. In preferred embodiments, ligands have functional utility for specific applications, e.g., for therapeutic, targeting, imaging, or drug delivery function(s). The terms “ligand”, “conjugate”, and “functional group” may be used interchangeably.

### DETAILED DESCRIPTION OF THE INVENTION

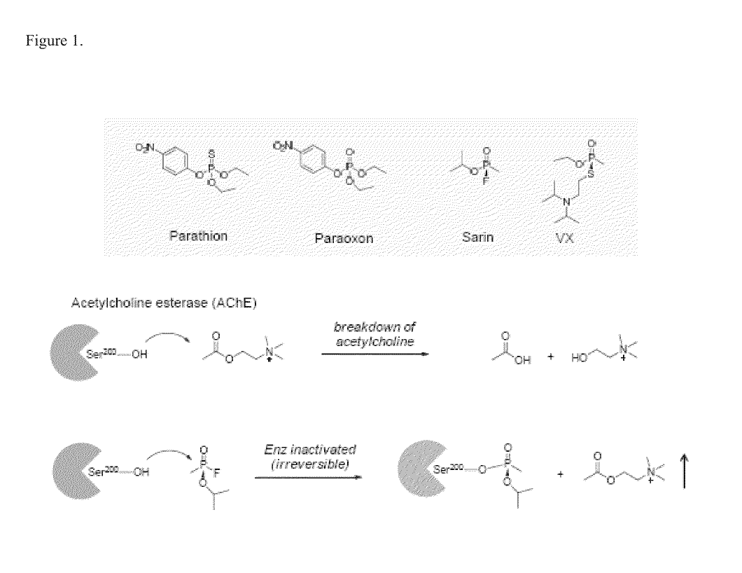

Reactive organophosphates (OP) collectively refer to a class of phosphate-based neurotoxic agents. They cause life threatening symptoms by covalently inhibiting acetylcholine esterase (AChE) (see, e.g., FIG. 1), and pose serious chemical threats to public health. Examples of such OP include nerve agents and those used commonly in civilian sector such as insecticides. While there are clinically approved antidotes for OP poisoning including pralidoxime (2-PAM) and obidoxime, the current therapy suffers from a major drawback because each drug affords only a very short duration of action primarily due to its rapid excretion.

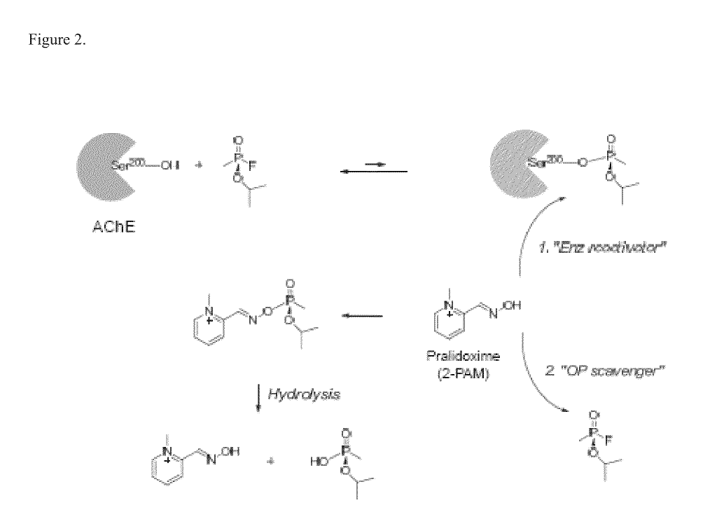

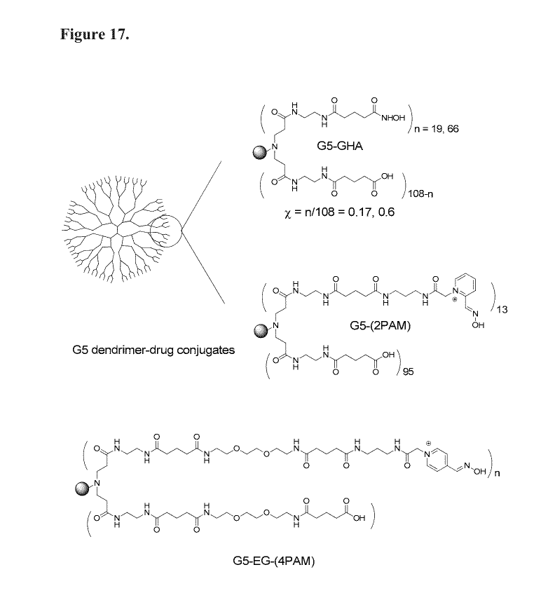

The present invention provides an improved nanotechnology based platform nanotechnology for the effective treatment of OP poisoning. In particular, experiments conducted during the course of developing embodiments for the present invention developed a multifunctional nanoscale particle derived from a dendrimer (e.g., polyamidoamine (PAMAM), Baker-Huang dendrimer), wherein the dendrimer is complexed with an OP antidote (e.g., pralidoxime (2-PAM) (4-PAM), obidoxime, trimedoxime, asoxime (HI-6), hydroxamate, and related analogs, salts and derivatives thereof) (see, e.g., FIG. 17). Such experiments demonstrated that the nanoparticle is rationally designed to have three distinct functions. First, the nanoparticle serves as a drug carrier by providing drug-binding cavities for OP antidotes (e.g., 2-PAM molecules) and enables to extend the duration of drug action through a sustained release mechanism. Second, the nanoparticle itself displays built-in therapeutic activity as an OP scavenger and the AChE reactivator (see, e.g., FIG. 2). Third, drug release within the nanopaticle is triggered by a feedback-regulated mechanism where the dendrimer drug carrier releases the OP antidote (e.g., 2-PAM) payloads in response to its OP scavenging action. Indeed, it was determined that the sustained release of the OP antidote in combination with the feedback release mechanism is a substantial improvement for the treatment of acute exposures to neurotoxic agents. In addition, such nanoparticles represent a suitable prophylactic option against OP poisoning.

Accordingly, the present invention provides novel therapeutic dendrimer conjugates configured for the treatment and/or prevention of organophosphate poisoning. In particular, the present invention provides dendrimers complexed with organophosphate poisoning antidotes (e.g., pralidoxime (2-PAM) (4-PAM), obidoxime, trimedoxime, asoxime (HI-6), hydroxamate, and related analogs, salts and derivatives thereof), compositions comprising such dendrimer conjugates, related methods of synthesizing such dendrimer conjugates, as well as systems and methods utilizing such dendrimer conjugates (e.g., in diagnostic and/or therapeutic settings (e.g., for the delivery of therapeutics, imaging, and/or targeting agents (e.g., in the treatment and/or prevention of organophosphate poisoning)).

The present invention is not limited to a particular manner of treating organophosphate poisoning with dendrimer conjugates. In some embodiments, the methods comprise administering to a subject (e.g., a mammal (e.g., human)) suffering from or susceptible to suffering from organophosphate poisoning a therapeutically effective amount of a composition comprising a dendrimer conjugate conjugated with an organophosphate poisoning antidote as described herein. The dendrimer conjugates of the present invention are not limited to a particular manner of treating organophosphate poisoning. In some embodiments, the dendrimer conjugates simultaneously serve as organophosphate scavengers (e.g., through locating, binding and hydrolyzing organophosphates) and reactivating AChE activity through disrupting organophosphates bound with AChE (see, e.g., FIG. 2). The methods for treating organophosphate poisoning are not limited to treating a particulate type of organophosphate and/or a particular type of exposure. Examples of organophosphates include, but are not limited to, insecticides (e.g., malathion, parathion, diazinon, fenthion, dichlorvos, chlorpyrifos, and ethion), nerve gases (e.g., soman, sarin, tabu, VX), ophthalmic agents (e.g., echothiophate, isoflurophate), antihelmintics (e.g., trichlorfon), and herbicides (e.g., tribufos (DEF). Exposure to any one of the above listed organophosphates can occur through, for example, inhalation, absorption, and ingestion, most commonly of food that has been treated with an organophosphate herbicide or insecticide. Exposure to these chemicals can occur, for example, at public buildings, war-zones, schools, residential areas, and in agricultural areas.

In some embodiments, the dendrimer conjugates configured for treating organophosphate poisoning are co-administered with one or more additional agents known to be effective in treating organophosphate poisoning. In some embodiments, the additional agent is an oxime (e.g., pralidoxime (2-PAM) (4-PAM), obidoxime, trimedoxime, asoxime (HI-6), hydroxamate, and related analogs, salts and/or derivatives thereof). In some embodiments, the additional agent is an anticholinergic agent (e.g., atropine, glycopyrrolate). In some embodiments, the additional agent is a benzodiazepine (e.g., diazepam).

The present invention also provides methods for prophylactically preventing organophosphate poisoning through administering to a subject at risk for organophosphate poisoning a therapeutically effective amount of a composition comprising a dendrimer conjugate conjugated with a therapeutic agent (e.g., an organophosphate poisoning antidote) as described herein. The dendrimer conjugates of the present invention are not limited to a particular manner of prophylactically preventing organophosphate poisoning. In some embodiments, the dendrimer conjugates serve as organophosphate scavengers (e.g., through locating, binding and hydrolyzing organophosphates) thereby prevening AChE inhibition.

The present invention is not limited to the use of particular types and/or kinds of dendrimers in developing/utilizing the dendrimer conjugates of the present invention. (e.g., a dendrimer conjugated with at least one functional group). Indeed, dendrimeric polymers have been described extensively (See, e.g., Tomalia, Advanced Materials 6:529 (1994); Angew, Chem. Int. Ed. Engl., 29:138 (1990); incorporated herein by reference in their entireties). Dendrimer polymers are synthesized as defined spherical structures typically ranging from 1 to 20 nanometers in diameter. Methods for manufacturing a G5 PAMAM dendrimer with a protected core are known (e.g., the protected core diamine is NH₂—CH₂—CH₂—NHPG) (U.S. patent application Ser. No. 12/403,179; herein incorporated by reference in its entirety). Molecular weight and the number of terminal groups increase exponentially as a function of generation (the number of layers) of the polymer. In some embodiments of the present invention, half generation PAMAM dendrimers are used. For example, when an ethylenediamine (EDA) core is used for dendrimer synthesis, alkylation of this core through Michael addition results in a half-generation molecule with ester terminal groups; amidation of such ester groups with excess EDA results in creation of a full-generation, amine-terminated dendrimer (Majoros et al., Eds. (2008) Dendrimer-based Nanomedicine, Pan Stanford Publishing Pte. Ltd., Singapore, p. 42). Different types of dendrimers can be synthesized based on the core structure that initiates the polymerization process.

The dendrimer core structures dictate several characteristics of the molecule such as the overall shape, density and surface functionality (See, e.g., Tomalia et al., Chem. Int. Ed. Engl., 29:5305 (1990)). Spherical dendrimers can have ammonia as a trivalent initiator core or ethylenediamine (EDA) as a tetravalent initiator core. Recently described rod-shaped dendrimers (See, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for GMP applications and in vivo usage.

Numerous U.S. Patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describes methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

PAMAM dendrimers are highly branched, narrowly dispersed synthetic macromolecules with well-defined chemical structures. PAMAM dendrimers can be easily modified and conjugated with multiple functionalities such as targeting molecules, imaging agents, and drugs (Thomas et al. (2007) Poly(amidoamine) Dendrimer-based Multifunctional Nanoparticles, in Nanobiotechnology: Concepts, Methods and Perspectives, Merkin, Ed., Wiley-VCH; herein incorporated by reference in its entirety). They are water soluble, biocompatible, and cleared from the blood through the kidneys (Peer et al. (2007) Nat. Nanotechnol. 2:751-760; herein incorporated by reference in its entirety) which eliminates the need for biodegradability. Because of these desirable properties, PAMAM dendrimers have been widely investigated for drug delivery (Esfand et al. (2001) Drug Discov. Today 6:427-436; Patri et al. (2002) Curr. Opin. Chem. Biol. 6:466-471; Kukowska-Latallo et al. (2005) Cancer Res. 65:5317-5324; Quintana et al. (2002) Pharmaceutical Res. 19:1310-1316; Thomas et al. (2005) J. Med. Chem. 48:3729-3735; each herein incorporated by reference in its entirety), gene therapy (KukowskaLatallo et al. (1996) PNAS 93:4897-4902; Eichman et al. (2000) Pharm. Sci. Technolo. Today 3:232-245; Luo et al. (2002) Macromol. 35:3456-3462; each herein incorporated by reference in its entirety), and imaging applications (Kobayashi et al. (2003) Bioconj. Chem. 14:388-394; herein incorporated by reference in its entirety).

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polyeation non-covalently coupled to the polynucleotide.

Classical preparation of PAMAM dendrimers is performed according to a typical divergent (building up the macromolecule from an initiator core) synthesis. It involves a two-step growth sequence that includes of a Michael addition of amino groups to the double bond of methyl acrylate (MA) followed by the amidation of the resulting terminal carbomethoxy, —(CO₂ CH₃) group, with ethylenediamine (EDA).

In the first step of this process, ammonia is allowed to react under an inert nitrogen atmosphere with MA (molar ratio: 1:4.25) at 47° C. for 48 hours. The resulting compound is referred to as generation=0, the star-branched PAMAM tri-ester. The next step involves reacting the tri-ester with an excess of EDA to produce the star-branched PAMAM tri-amine (G=0). This reaction is performed under an inert atmosphere (nitrogen) in methanol and requires 48 hours at 0° C. for completion. Reiteration of this Michael addition and amidation sequence produces generation=1.

Preparation of this tri-amine completes the first full cycle of the divergent synthesis of PAMAM dendrimers. Repetition of this reaction sequence results in the synthesis of larger generation (G=1-5) dendrimers (i.e., ester- and amine-terminated molecules, respectively). For example, the second iteration of this sequence produces generation 1, with an hexa-ester and hexa-amine surface, respectively. The same reactions are performed in the same way as for all subsequent generations from 1 to 9, building up layers of branch cells giving a core-shell architecture with precise molecular weights and numbers of terminal groups as shown above. Carboxylate-surfaced dendrimers can be produced by hydrolysis of ester-terminated PAMAM dendrimers, or reaction of succinic anhydride with amine-surfaced dendrimers (e.g., full generation PAMAM, POPAM or POPAM-PAMAM hybrid dendrimers).

Various dendrimers can be synthesized based on the core structure that initiates the polymerization process. These core structures dictate several important characteristics of the dendrimer molecule such as the overall shape, density, and surface functionality (See, e.g., Tomalia et al., Angew. Chem. Int. Ed. Engl., 29:5305 (1990)). Spherical dendrimers derived from ammonia possess trivalent initiator cores, whereas EDA is a tetra-valent initiator core. Recently, rod-shaped dendrimers have been reported which are based upon linear poly(ethyleneimine) cores of varying lengths the longer the core, the longer the rod (See, e.g., Yin et al., J. Am. Chem. Soc., 120:2678 (1998)).

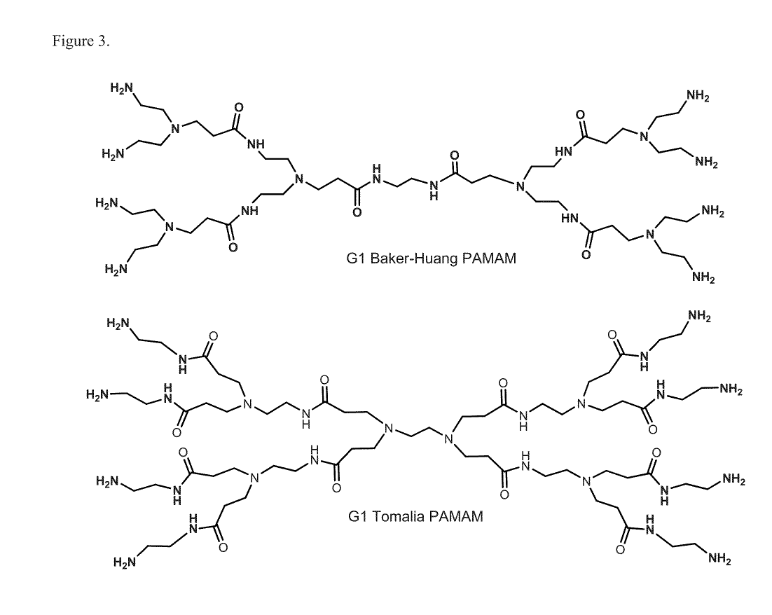

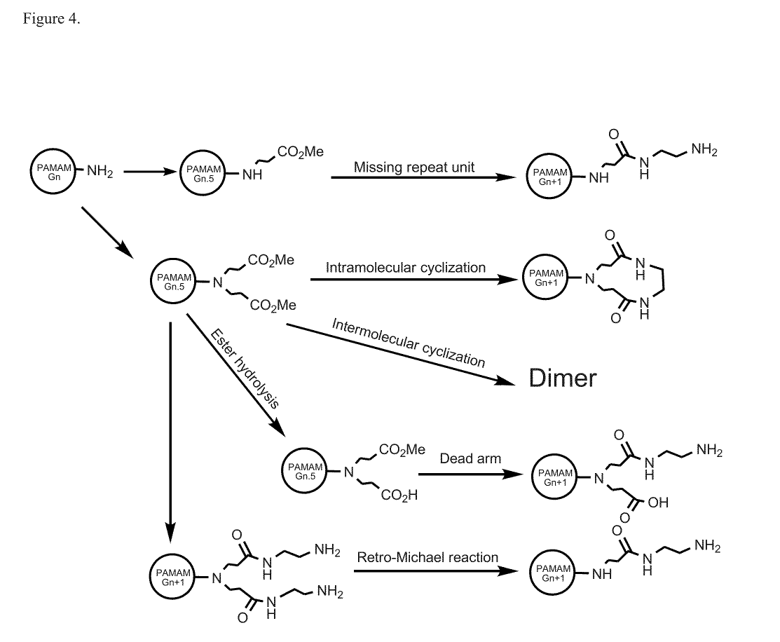

In some embodiments, the dendrimers are “Baker-Huang dendrimers” (see, e.g., U.S. Provisional Patent Application No. 61/251,244 and International Patent Application No. PCT/US2010/051835; each herein incorporated by reference in its entirety). Baker-Huang dendrimers are structurally distinct from classical PAMAM dendrimers (e.g., Tomalia PAMAM dendrimers). FIG. 3 shows a comparison of an embodiment of a generation 1 (G1) Baker-Huang PAMAM dendrimer and a classical Tomalia G1 PAMAM dendrimer. While both dendrimers are poly-amido-amine (PAMAM) dendrimers, they are structurally distinct. The Tomalia PAMAM dendrimer structure includes (listed in order from the core to the surface) iterative repeats of tertiary amines followed by amide bonds with ethylene groups intervening between the tertiary amines and amide bonds. In contrast, with Baker-Huang PAMAM dendrimers the order of iterative repeats from core to surface changes to amide bonds first, followed by tertiary amines, again with ethylene groups intervening between the amide bond and tertiary amines. While structural similarities exist between classical PAMAM dendrimers and Baker-Huang PAMAM dendrimers, there are also structural distinctions. Notably, Baker-Huang PAMAM dendrimers have fewer amide bonds and a less crowded interior (core) (see, e.g., FIG. 3). In particular, the interior core of Baker-Huang dendrimers permit increased interior space and less steric hindrance, which finds use, e.g., for encapsulation of agents or attachment of additional functional ligands (e.g., therapeutic agents, imaging agents, trigger agents, targeting agents).

The present invention is not limited to a particular method for synthesizing Baker-Huang PAMAM dendrimers. In certain embodiments, the present invention provides novel dendrimer branching units for generating Baker-Huang PAMAM dendrimers. FIG. 4 shows one embodiment of an AB₂ branch unit. In the terminology used herein regarding branch units, A may comprise a carboxylic acid, and B may comprise a protected amine. In some synthesis method embodiments, amide bond formation is utilized for generation growth of dendrimers constructed using AB₂ branch unit embodiments of the present invention. For example, for a EDA core, the AB₂ branch unit embodiment shown in FIG. 4 reacts at both end of the EDA molecule, thereby forming a G0 dendrimer (e.g., Baker-Huang PAMAM G0 dendrimer). In the embodiment shown in FIG. 4, the selection of trifluoroacetamide as a protection group for the primary amine has several advantages. For example, trifluoroacetamide is very stable under acidic conditions; therefore, the solubility of the branch unit embodiment in organic solvent is desirable because the coupling reactions may be performed in organic solvent. Additionally, trifluoroacetamide can be removed under mild conditions (see, e.g., U.S. Provisional Patent Application No. 61/251,244 and International Patent Application No. PCT/US2010/051835; each herein incorporated by reference in its entirety).

As recited above, the present invention provides dendrimers complexed with organophosphate poisoning, compositions comprising such dendrimer conjugates, related methods of synthesizing such dendrimer conjugates, as well as systems and methods utilizing such dendrimer conjugates (e.g., in diagnostic and/or therapeutic settings (e.g., for the delivery of therapeutics, imaging, and/or targeting agents (e.g., in the treatment of organophosphate poisoning)).

In some embodiments, the dendrimer is conjugated with one or more therapeutic agents. The present invention is not limited to particular therapeutic agent. In some embomdiments, the therapeutic agent includes organophosphate poisoning antidotes. In some embodiments, the organophosphate poisoning antidotes include, but are not limited to, an oxime (e.g., pralidoxime (2-PAM) (4-PAM), obidoxime, trimedoxime, asoxime (HI-6), hydroxamate, and related analogs, salts and/or derivatives thereof). In some embodiments, the therapeutic agent is an anticholinergic agent (e.g., atropine, glycopyrrolate). In some embodiments, the therapeutic agent is a benzodiazepine (e.g., diazepam). In some embodiments, the therapeutic agent may be any agent selected from the group comprising, but not limited to, a pain relief agent, a pain relief agent antagonist, a chemotherapeutic agent, an anti-oncogenic agent, an anti-angiogenic agent, a tumor suppressor agent, an anti-microbial agent, or an expression construct comprising a nucleic acid encoding a therapeutic protein (see., U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Serial Nos. 61/562,767, 61/568,521, 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/054202, PCT/US2010/050893, PCT/U52010/050893, PCT/US2010/042556, PCT/US2001/015204, PCT/US2005/030278, PCT/US2009/069257, PCT/US2009/036992, PCT/US2009/059071, PCT/US2007/015976, and PCT/US2008/061023, each herein incorporated by reference in their entireties).

In some embodiments, the dendrimer is conjugated with one or more targeting agents. In some embodiments, targeting agents are conjugated to the dendrimers (e.g., Baker-Huang PAMAM dendrimers) for delivery of the dendrimers to desired body regions (e.g., to the central nervous system (CNS)) (e.g., to an organophosphate). The targeting agents are not limited to targeting specific body regions. In some embodiments, the targeting agents target the central nervous system (CNS). In some embodiments, where the targeting agent is specific for the CNS, the targeting agent is transferrin (see, e.g., Daniels, T. R., et al., Clinical Immunology, 2006. 121(2): p. 159-176; Daniels, T. R., et al., Clinical Immunology, 2006. 121(2): p. 144-158; each herein incorporated by reference in their entireties). Transferrin has been utilized as a targeting vector to transport, for example, drugs, liposomes and proteins across the BBB by receptor mediated transcytosis (see, e.g., Smith, M. W. and M. Gumbleton, Journal of Drug Targeting, 2006. 14(4): p. 191-214; herein incorporated by reference in its entirety). In some embodiments, the targeting agents target neurons within the central nervous system (CNS). In some embodiments, where the targeting agent is specific for neurons within the CNS, the targeting agent is a synthetic tetanus toxin fragment (e.g., a 12 amino acid peptide (Tet 1) (HLNILSTLWKYR)) (see, e.g., Liu, J. K., et al., Neurobiology of Disease, 2005. 19(3): p. 407-418; herein incorporated by reference in its entirety).

In some embodiments, the targeting agent is a moiety that has affinity for a tumor associated factor. For example, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)). In some embodiments, the targeting agent is an antibody, receptor ligand, hormone, vitamin, or antigen. However, the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, or VEGFR. In some embodiments, the receptor ligand is folic acid.

The present invention is not limited to cancer and/or tumor targeting agents. Indeed, dendrimers of the present invention can be targeted (e.g., via a linker conjugated to the dendrimer wherein the linker comprises a targeting agent) to a variety of target cells or tissues (e.g., to a biologically relevant environment) via conjugation to an appropriate targeting agent. For example, in some embodiments, the targeting agent is a moiety that has affinity for an inflammatory factor (e.g., a cytokine or a cytokine receptor moiety (e.g., TNF-α receptor)). In some embodiments, the targeting agent is a sugar, peptide, antibody or antibody fragment, hormone, hormone receptor, or the like.

In some embodiments of the present invention, the targeting agent includes, but is not limited to an antibody, receptor ligand, hormone, vitamin, and antigen, however, the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

In some embodiments, the dendrimer is conjugated with one or more imaging agents. The dendrimer conjugates are not limited to particular imaging agents. In some embodiments, the imaging agent is selected from the group consisting of a fluorescing entity (e.g., fluorescein isothiocyanate (FITC)), 6-TAMARA, acridine orange, and cis-parinaric acid. In some embodiments, the imaging agent comprises a radioactive label including, but not limited to ¹⁴C, ³⁶Cl, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb.

In some embodiments, conjugation of a functional group (e.g., imaging agents, targeting agents, therapeutic agents, locking agents, etc.) with the dendrimer is accomplished via a covalent attachment with the dendrimer (e.g., via a stable amide linkage with the dendrimer) (e.g., via a stable amine linkage with the dendrimer).

In some embodiments, conjugation of a functional group (e.g., imaging agents, targeting agents, therapeutic agents, locking agents, etc.) with the dendrimer is accomplished with a linker and/or a trigger agent. The present invention is not limited to particular manner of conjugation of a functional group with a dendrimer via a linker and/or trigger agent.

In some embodiments, a dendrimer (e.g., a Baker-Huang PAMAM dendrimer) conjugated to a linker that is conjugated to a functional group (e.g., therapeutic agent, imaging agent, targeting agent, triggering agent) decreases the number of conjugation steps required to form a dendrimer conjugate (e.g., a dendrimer conjugated to a targeting agent, imaging agent, therapeutic agent and/or triggering agent). For example, in some embodiments, the present invention provides a customizable dendrimer (e.g., a Baker-Huang PAMAM dendrimer) wherein one or a plurality of linkers (e.g. attached to one or a plurality of targeting agents, triggering agents and/or therapeutic agents) are conjugated to the dendrimer, thereby decreasing the number of conjugation steps used to form a dendrimer conjugate (e.g., versus a dendrimer that is conjugated to a targeting moiety in one step and that is separately conjugated to a linker (e.g., comprising a therapeutic agent, imaging agent, triggering agent or other moiety) in an additional conjugation step). In some embodiments, a linker conjugated to one or more agents (e.g., therapeutic agents, imaging agents, targeting agents, triggering agents) is conjugated to one or more additional moieties including, but not limited to, a therapeutic agent, a triggering agent, an imaging agent, a triggering agent, etc. Thus, in some embodiments, the present invention provides a dendrimer with increased load capacity (e.g., increased load of therapeutic, imaging agent, etc. on the dendrimer). In some embodiments, two or more linkers (e.g., conjugated to one or a plurality of therapeutic agents) are conjugated to a dendrimer via the same or different linkage (e.g., covalent linkage).

Several different schemes have been evaluated for generating dendrimer conjugates wherein a dendrimer is conjugated to one or more linkers that comprise multiple sites for binding (e.g., covalent binding) moieties. For example, in one embodiment, a linker may comprise a chemical structure that allows, for example, conjugation of a targeting moiety and a therapeutic compound to the linker Thus, in some embodiments, a dendrimer conjugate of the present invention (e.g., a Baker-Huang PAMAM dendrimer conjugate) permits control of the stoichiometry between targeting agent and therapeutic compound (e.g., generation of one to one ratio, two to one ratio, one to two ratio, one to three ratio etc. between targeting and therapeutic moieties).

In some embodiments, a dendrimer (e.g., a Baker-Huang dendrimer) conjugated to a linker that is conjugated to a functional group (e.g., targeting agent and/or therapeutic agent) comprises a linker that is configured to be irreversibly degraded (e.g., that is non-reversible (e.g., that permits drug delivery at the correct time and/or at the correct place)).

In some embodiments, the present invention provides dendrimer molecules conjuguated to one or more therapeutic agents configured for controlled and/or sustained release of the therapeutic agents (e.g., through use of targeting agents, linking agents, and/or trigger agents conjugated to the dendrimer and/or therapeutic agent). In some embodiments, the therapeutic agent conjugated to the dendrimer is active upon administration to a subject. In some embodiments, sustained release (e.g., slow release over a period of 24-48 hours) of the therapeutic agent is accomplished through conjugating the therapeutic agent to the dendrimer through, for example, a linkage agent connected to a trigger agent that slowly degrades in a biological system (e.g., amide linkage, ester linkage, ether linkage). In some embodiments, constitutively active release of the therapeutic agent is accomplished through conjugating the therapeutic agent to the dendrimer through, for example, a linkage agent connected to a trigger agent that renders the therapeutic agent constitutively active in a biological system (e.g., amide linkage, ether linkage). In some embodiments, the dendrimers conjugated to one or more therapeutic agents are simultaneously configured for sustained release (e.g., a slow release mechanism that achieves therapeutic concentrations over a period of, for example, 24-48 hours) of the therapeutic agent.

In some embodiments, the dendrimer conjugates comprise i) a targeting agent that enables the conjugate to cross the blood-brain-barrier (BBB) and target neurons, ii) a locking agent (e.g., a re-dox locking module) to prevent the dendrimer conjugate from diffusing back across the BBB, and iii) a therapeutic agent (e.g., an organophosphate poisoning antidote). The dendrimer conjugates are not limited to particular targeting agents. In some embodiments, the targeting agent for CNS targeting through crossing the BBB is transferrin (see, e.g., Daniels, T. R., et al., Clinical Immunology, 2006. 121(2): p. 159-176; Daniels, T. R., et al., Clinical Immunology, 2006. 121(2): p. 144-158; each herein incorporated by reference in their entireties). In some embodiments, the targeting agent for neuron targeting is a 12 amino acid peptide (Tet 1) (see, e.g., Liu, J. K., et al., Neurobiology of Disease, 2005. 19(3): p. 407-418; herein incorporated by reference in its entirety). The dendrimer conjugates are not limited to particular locking agents. In some embodiments, the locking agent for locking the dendrimer conjugate within the CNS is the 1,4-dihydrotrigonelline [symbol] trigonelline (coffearine) re-dox system where the lipophilic 1,4-dihydro form (L) is converted in vivo to the hydrophilic quaternary form (L⁺) by oxidation to prevent the dendrimer conjugate from diffusing back into the circulation (see, e.g., Bodor, N. and P. Buchwald, Drug Discovery Today, 2002. 7(14): p. 766-774; herein incorporated by reference in its entirety). In some embodiments, the dendrimer conjugate device is eliminated from the CNS (e.g., because of acquired hydrophilicity due to loss of the quaternary form).

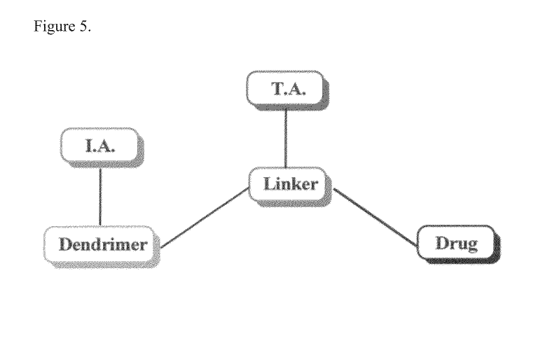

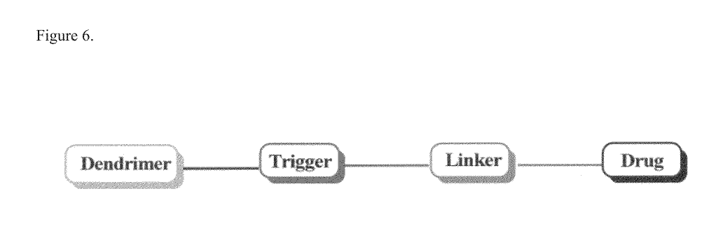

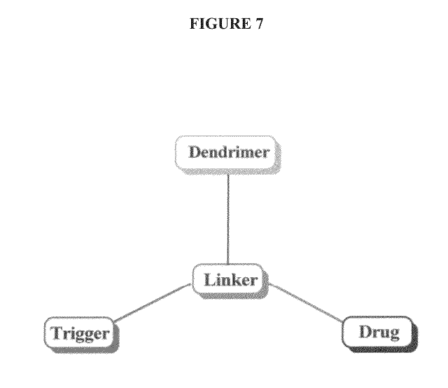

In some embodiments, the present invention provides a dendrimer conjugate as shown in FIG. 5. For example, FIG. 5 shows a targeting agent (T.A.) conjugated to a linker that is also conjugated to a drug, wherein the linker conjugated to a drug and targeting agent is conjugated to a dendrimer conjugated to an imaging agent (I.A.). In some embodiments, the present invention provides a dendrimer conjugate as shown in FIGS. 6 and 7. In particular, a dendrimer conjugate as shown in FIG. 6 comprises a dendrimer (e.g., a G5 PAMAM dendrimer conjugated to an imaging agent (e.g., FITC) and/or targeting agent) conjugated to a trigger molecule that is conjugated to a linker that is conjugated to a therapeutic. A dendrimer conjugate as shown in FIG. 7 comprises a dendrimer (e.g., a G5 PAMAM dendrimer conjugated to an imaging agent (e.g., FITC) and/or targeting agent) conjugated to a linker that is conjugated to a trigger and to a therapeutic moiety. The conjugates of FIGS. 6 and 7 are configured to be non-toxic to normal cells. For example, the conjugates are configured in such a way so as to release their therapeutic agent only at a specific, targeted site (e.g., through activation of a trigger molecule that in to leads to release of the therapeutic agent) For example, once a conjugate arrives at a target site in a subject (e.g., a tumor, or a site of inflammation), components in the target site (e.g., a tumor associated factor, or an inflammatory or pain associated factor) interacts with the trigger moiety thereby initiating cleavage of this unit from the linker. In some embodiments, once the trigger is cleaved from the linker (e.g., by a target associated moiety) the linker proceeds through spontaneous chemical breakdown thereby releasing the therapeutic agent at the target site (e.g., in its active form). The present invention is not limited to any particular target associated moiety (e.g., that interacts with and initiates cleavage of a trigger). In some embodiments, the target associated moiety is a tumor associated factor (e.g., an enzyme (e.g., glucuronidase and/or plasmin), a cathepsin, a matrix metalloproteinase, a hormone receptor (e.g., integrin receptor, hyaluronic acid receptor, luteinizing hormone-releasing hormone receptor, etc.), cancer and/or tumor specific DNA sequence), an inflammatory associated factor (e.g., chemokine, cytokine, etc.) or other moiety.

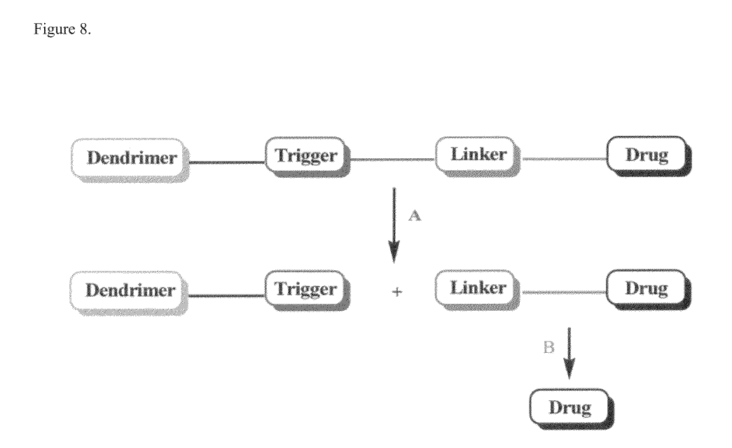

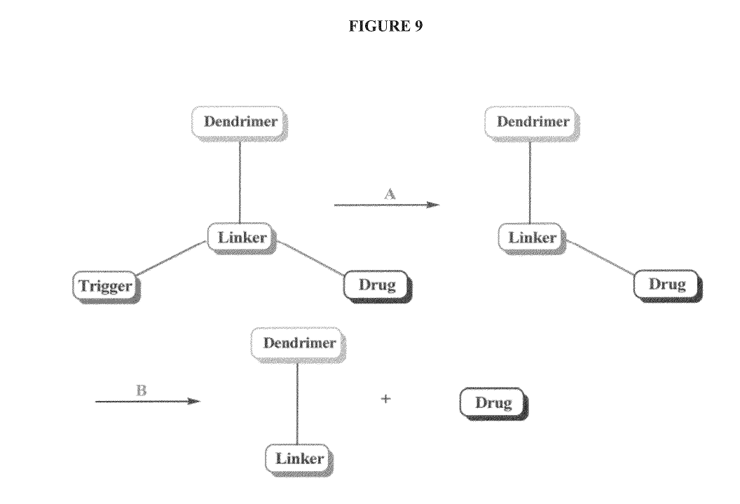

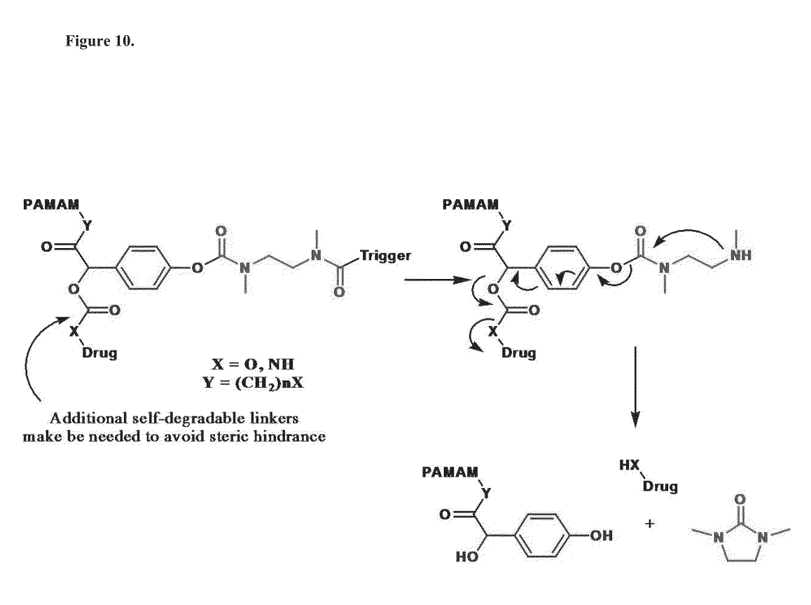

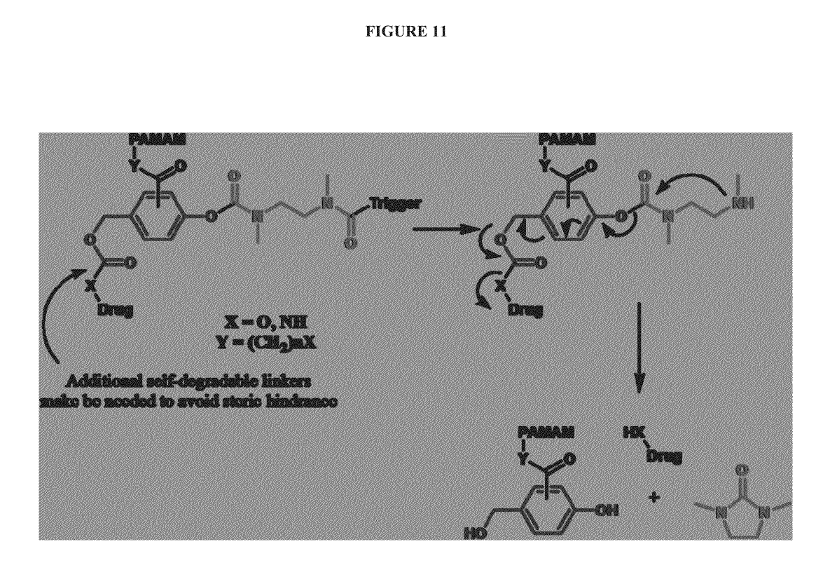

Although an understanding of a mechanism of action is not necessary to practice the present invention, and the present invention is not limited to any particular mechanism of action, in some embodiments, a dendrimer conjugate as described in FIGS. 6 and 7 provides a therapeutic to a site by a mechanism as shown in FIGS. 8 and 9. For example, as shown in FIG. 8, a dendrimer conjugate comprising a dendrimer (e.g., a G5 PAMAM dendrimer (e.g., a Baker-Huang PAMAM dendrimer) conjugated to an imaging agent (e.g., FITC) and/or targeting agent) conjugated to a trigger molecule that is conjugated to a linker that is conjugated to a therapeutic (A) interacts with a target associated moiety thereby activating the trigger and initiating cleavage of same, releasing the linker therapeutic drug conjugate. Once cleavage of the trigger occurs, the linker (B) proceeds through a spontaneous chemical breakdown at the target site, releasing (e.g., irreversibly releasing) the therapeutic drug at the target site. In some embodiments, as shown in FIG. 9, a dendrimer conjugate comprising a dendrimer (e.g., a G5 PAMAM dendrimer (e.g., Baker-Huang dendrimer) conjugated to an imaging agent (e.g., FITC) and/or targeting agent) conjugated to a linker that is conjugated to a trigger and to a therapeutic moiety (A) interacts with a target associated moiety thereby activating the trigger and initiating cleavage of same, releasing a dendrimer-linker-therapeutic moiety from the trigger. Once cleavage of the trigger occurs, the linker (B) proceeds through a spontaneous chemical breakdown (e.g., to a point where the therapeutic drug is released from the dendrimer linker conjugate) at the target site, releasing (e.g., irreversibly releasing) the therapeutic drug at the target site. In some embodiments, cleavage of the trigger and subsequent linker breakdown is not necessary to deliver the therapeutic drug to the target site. Several design processes for generating a dendrimer conjugate comprising a trigger are shown in FIGS. 10, 11, 12, and 13. In some embodiments, one or more amino groups present on the dendrimer are linked (e.g., through a covalent bond) to one or more targeting agents (e.g., folic acid) and/or imaging agents (e.g., FITC) (e.g., as described in U.S. Pat. Nos. 6,471,968 and 7,078,461; U.S. Patent Pub. Nos. 20020165179 and 20070041934 and WO 06/033766, each of which is hereby incorporated by reference in its entirety for all purposes).

The dendrimer conjugates of the present invention are not limited to uses within particular settings. Indeed, the dendrimer conjugates of the present invention may be used in any setting requiring treatment (e.g., battlefield, ambulance, hospital, clinic, rescue, etc.). In addition, the present invention contemplates dendrimer conjugates comprising one or more theapeutic agent prodrugs and/or therapeutic agent antagonist prodrugs developed for site specific conversion to drug based on tumor associated factors (e.g., hypoxia and pH, tumor-associated enzymes, and/or receptors). In some embodiments, dendrimer conjugates of the present invention are configured such that a prodrug (e.g., therapeutic agent prodrug, therapeutic agent antagonist prodrug) is conjugated to a linker that is further conjugated to a targeting moiety (e.g., that targets the conjugate to a particular body region (e.g., CNS)). Although an understanding of the mechanism is not necessary for the present invention, and the present invention is not limited to any particular mechanism of action, in some embodiments, a trigger component serves as a precursor for site-specific activation. For example, in some embodiments, once the trigger recognizes a particular condition (e.g., hypoxia), cleavage and/or processing of the trigger is induced, thereby releasing the therapeutic agent and/or therapeutic antagonist.

The present invention is not limited to a particular trigger agent or to any particular cleavage and/or processing of the trigger agent. In some embodiments, the present invention provides therapeutic agents and/or therapeutic agent antagonists coupled to dendrimers with a linkage agent connected to a trigger agent that slowly degrades in a biological system (e.g., amide linkage, ester linkage, ether linkage).

In some embodiments, the present invention provides a dendrimer conjugate comprising a trigger agent that is sensitive to (e.g., is cleaved by) hypoxia. Hypoxia is a feature of several disease states, including cancer, inflammation and rheumatoid arthritis, as well as an indicator of respiratory depression (e.g., resulting from analgesic drugs). Advances in the chemistry of bioreductive drug activation have led to the design of various hypoxia-selective drug delivery systems in which the pharmacophores of drugs are masked by reductively cleaved groups. In some embodiments, a dendrimer conjugate of the present invention (e.g., a Baker-Huang PAMAM dendrimer conjugate) utilizes a quinone, N-oxide and/or (hetero)aromatic nitro groups. For example, a quinone present in a dendrimer conjugate of the present invention is reduced to phenol under hypoxia conditions, with spontaneous formation of lactone that serves as a driving force for drug release. In some embodiments, a heteroaromatic nitro compound present in a dendrimer conjugate of the present invention is reduced to either an amine or a hydroxylamine, thereby triggering the spontaneous release of a therapeutic agent/drug. In some embodiments, the present invention provides therapeutic agents and/or therapeutic agent antagonists coupled to dendrimers with a linkage agent connected to a trigger agent that degrades upon detection of reduced pO2 concentrations (e.g., through use of a re-dox linker).

The concept of prodrug systems in which the pharmacophores of drugs are masked by reductively cleavable groups has been widely explored by many research groups and pharmaceutical companies (see, e.g., Beall, H. D., et al., Journal of Medicinal Chemistry, 1998. 41(24): p. 4755-4766; Ferrer, S., D. P. Naughton, and M. D. Threadgill, Tetrahedron, 2003. 59(19): p. 3445-3454; Naylor, M. A., et al., Journal of Medicinal Chemistry, 1997. 40(15): p. 2335-2346; Phillips, R. M., et al., Journal of Medicinal Chemistry, 1999. 42(20): p. 4071-4080; Zhang, Z., et al., Organic & Biomolecular Chemistry, 2005. 3(10): p. 1905-1910; each of which are herein incorporated by reference in their entireties). Several such hypoxia activated prodrugs have been advanced to clinical investigations, and work in relevant oxygen concentrations to prevent cerebral damage. The present invention is not limited to particular hypoxia activated trigger agents. In some embodiments, the hypoxia activated trigger agents include, but are not limited to, indoquinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770; each herein incorporated by reference in their entireties).

In some embodiments, the present invention provides a dendrimer conjugate (e.g., a Baker-Huang dendrimer conjugate) comprising a trigger agent that is sensitive to (e.g., is cleaved by) and/or that associates with a tumor associated enzyme. In some embodiments, the present invention provides a dendrimer conjugate comprising a trigger that is sensitive to (e.g., is cleaved by) and/or that associates with a glucuronidase. Glucuronic acid can be attached to several anticancer drugs via various linkers. These anticancer drugs include, but are not limited to, doxorubicin, paclitaxel, docetaxel, 5-fluorouracil, 9-aminocamtothecin, as well as other drugs under development. These prodrugs are generally stable at physiological pH and are significantly less toxic than the parent drugs. In some embodiments, dendrimer conjugates comprising anticancer prodrugs find use for treating necrotic tumors (e.g., that liberate β-glucuronidase) or for ADEPT with antibodies that can deliver β-glucuronidase to target tumor cells.

In some embodiments, the present invention provides a dendrimer conjugate comprising a trigger agent that is sensitive to (e.g., is cleaved by) and/or that associates with brain enzymes. For example, trigger agents such as indolequinone are reduced by brain enzymes such as, for example, diaphorase (see, e.g., Damen, E. W. P., et al., Bioorganic & Medicinal Chemistry, 2002. 10(1): p. 71-77; herein incorporated by reference in its entirety). For example, in such embodiments, the antagonist is only active when released during hypoxia to prevent respiratory failure.

In some embodiments, the present invention provides a dendrimer conjugate comprising a trigger agent that is sensitive to (e.g., is cleaved by) and/or that associates with a protease. The present invention is not limited to any particular protease. In some embodiments, the protease is a cathepsin. In some embodiments, a trigger comprises a Lys-Phe-PABC moiety (e.g., that acts as a trigger). In some embodiments, a Lys-Phe-PABC moiety linked to doxorubicin, mitomycin C, and paclitaxel are utilized as a trigger-therapeutic conjugate in a dendrimer conjugate provided herein (e.g., that serve as substrates for lysosomal cathepsin B or other proteases expressed (e.g., overexpressed) in tumor cells. In some embodiments, utilization of a 1,6-elimination spacer/linker is utilized (e.g., to permit release of therapeutic drug post activation of trigger).

In some embodiments, the present invention provides a dendrimer conjugate comprising a trigger agent that is sensitive to (e.g., is cleaved by) and/or that associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the present invention provides a dendrimer conjugate comprising a trigger agent that is sensitive to (e.g., is cleaved by) and/or that associates with a matrix metalloproteases (MMPs). In some embodiments, the present invention provides a dendrimer conjugate comprising a trigger that is sensitive to (e.g., is cleaved by) and/or that associates with β-Lactamase (e.g., a β-Lactamase activated cephalosporin-based prodrug).

In some embodiments, the present invention provides a dendrimer conjugate comprising a trigger agent that is sensitive to (e.g., is cleaved by) and/or activated by a receptor (e.g., expressed on a target cell (e.g., a tumor cell)). Thus, in some embodiments, a dendrimer conjugate comprises a receptor binding motif conjugated to a therapeutic agent (e.g., cytotoxic drug) thereby providing target specificity. Examples include, but are not limited to, a dendrimer conjugate comprising a prodrug (e.g., of doxorubicin and/or paclitaxel) targeting integrin receptor, a hyaluronic acid receptor, and/or a hormone receptor.

In some embodiments, the present invention provides a dendrimer conjugate comprising a trigger agent that is sensitive to (e.g., is cleaved by) and/or activated by a nucleic acid. Nucleic acid triggered catalytic drug release can be utilized in the design of chemotherapeutic agents. Thus, in some embodiments, disease specific nucleic acid sequence is utilized as a drug releasing enzyme-like catalyst (e.g., via complex formation with a complimentary catalyst-bearing nucleic acid and/or analog). In some embodiments, the release of a therapeutic agent is facilitated by the therapeutic component being attached to a labile protecting group, such as, for example, cisplatin or methotrexate being attached to a photolabile protecting group that becomes released by laser light directed at cells emitting a color of fluorescence (e.g., in addition to and/or in place of target activated activation of a trigger component of a dendrimer conjugate). In some embodiments, the therapeutic device also may have a component to monitor the response of the tumor to therapy. For example, where a therapeutic agent of the dendrimer induces apoptosis of a target cell (e.g., a cancer cell (e.g., a prostate cancer cell)), the caspase activity of the cells may be used to activate a green fluorescence. This allows apoptotic cells to turn orange, (combination of red and green) while residual cells remain red. Any normal cells that are induced to undergo apoptosis in collateral damage fluoresce green.

In some embodiments, the present invention provides a dendrimer conjugate comprising a linker that connects to a therapeutic compound. In some embodiments, the linker is configured such that its decomposition leads to the liberation (e.g., non-reversible liberation) of the therapeutic agent (e.g., at the target site (e.g., site of tumor, CNS, and/or inflammatory site)). The linker may influence multiple characteristics of a dendrimer conjugate including, but not limited to, properties of the therapeutic agent (e.g., stability, pharmacokinetic, organ distribution, bioavailability, and/or enzyme recognition (e.g., when the therapeutic agent (e.g., prodrug)) is enzymatically activated)).

In some embodiments, the linker is an elimination linker. For example, in some embodiments, in a dendrimer conjugate of the present invention (e.g., a Baker-Huang PAMAM dendrimer conjugate), when a trigger is cleaved (e.g., enzymatically and/or chemically), a phenol or an aniline promotes a facile 1,4 or 1,6 elimination, followed by release of a CO₂ molecule and the unmasked therapeutic agent (e.g., drug). In some embodiments, a dendrimer conjugate of the present invention utilizes this configuration and/or strategy to mask one or more hydroxyl groups and/or amino groups of the therapeutic agents. In some embodiments, a linker present within a dendrimer conjugate of the present invention is fine tuned (e.g., to optimize stability and/or drug release from the conjugate). For example, the sizes of the aromatic substituents can be altered (e.g., increased or decreased) and/or alkyl substitutions at the benzylic position may be made to alter (e.g., increase or decrease) degradation of the linker and/or release of the therapeutic agent (e.g., prodrug). In some embodiments, elongated analogs (e.g., double spacers) are used (e.g., to decrease steric hindrance (e.g., for large therapeutic agents)). In some embodiments, a dendrimer conjugate of the present invention comprises an enol based linker (e.g., that undergoes an elimination reaction to release therapeutic agent (e.g., prodrug)).

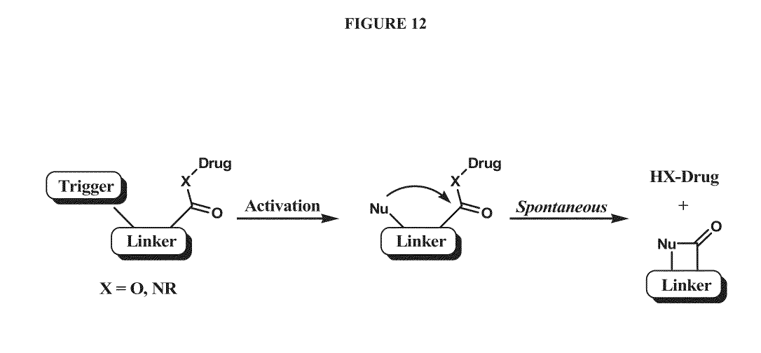

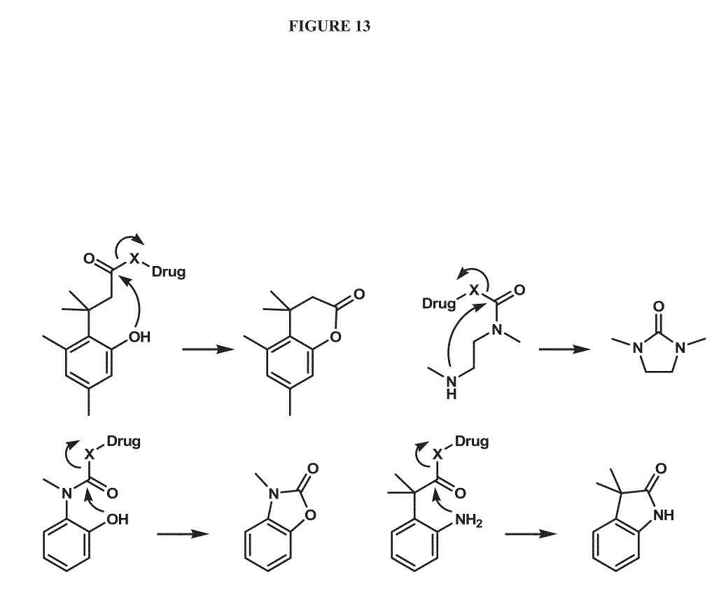

In some embodiments, the linker is a cyclization based linker. For example, one configuration for this approach is shown in FIG. 12. A nucleophilic group (e.g., OH or NHR) that becomes available once the trigger is cleaved attacks the carbonyl of the C(O)X-Therapeutic agent/drug (e.g., thereby leading to release of therapeutic agent-XH) and thereby to quickly release the Drug-XH. In some embodiments, a driving force that permits the reaction to reach completion is the stability of the cyclic product. In some embodiments, a cyclization based linker of a dendrimer conjugate of the present invention include, but are not limited to, those shown in FIG. 13.

In some embodiments, a dendrimer conjugate (e.g., a Baker-Huang PAMAM dendrimer conjugate) of the present invention comprises a combination of one or more linkers. For example, in some embodiments, a dendrimer conjugate comprises a combination of two or more elimination linkers. In some embodiments, a dendrimer conjugate of the present invention comprises two or more cyclization linkers. In some embodiments, a dendrimer conjugate of the present invention comprises a one or more elimination linkers and one or more cyclization linkers, or a combination of one or more different types of linkers described herein.

In some embodiments, a dendrimer conjugate of the present invention comprises branched self-elimination linkers. Thus, in some embodiments, use of branched linkers provides a conjugate that can present increased concentrations of a therapeutic agent to a target site (e.g., inflammatory site, tumor site, etc.).

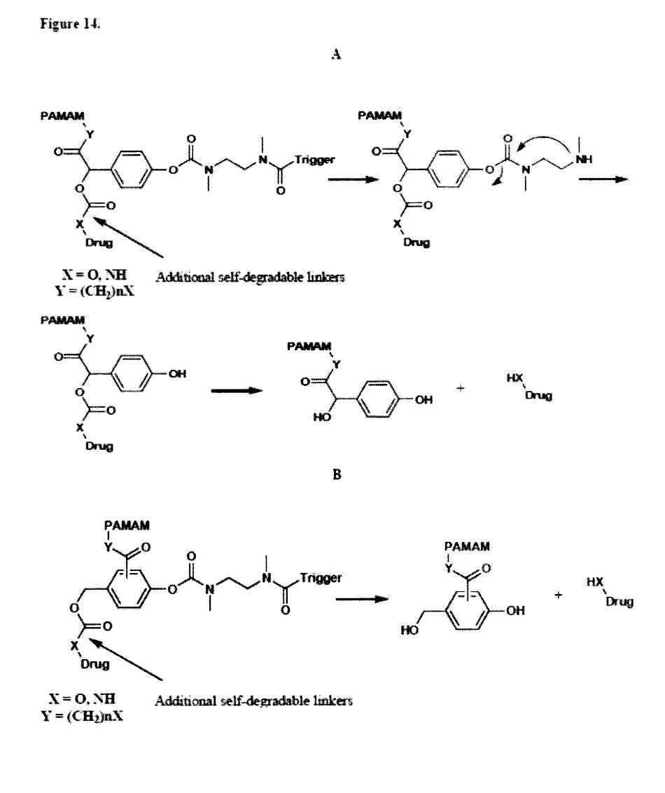

In some embodiments, a dendrimer conjugate of the present invention is generated by a process comprising conjugating a pre-formed tripartite piece (e.g., trigger, linker, and therapeutic agent) to a dendrimer (e.g., a G5 Baker-Huang PAMAM dendrimer (e.g., conjugated to one or more different types of agents (e.g., imaging agent)). In some embodiments, linkage between a tripartite piece and a dendrimer comprises a non-cleavable bond (e.g., an ether or an amide bond (e.g., thereby decreasing unwanted activation of a trigger and/or degradation of a linker and/or release of therapeutic drug). In some embodiments, a linker (e.g., linear or other type of linker described herein) is utilized to attach a tripartite moiety (e.g., trigger, linker, and therapeutic agent) to a dendrimer (e.g., in order to increase drug release, decrease steric hindrance, and/or increase stability of the dendrimer). For example, in some embodiments, the present invention provides a dendrimer conjugate as shown in FIGS. 14A-B.

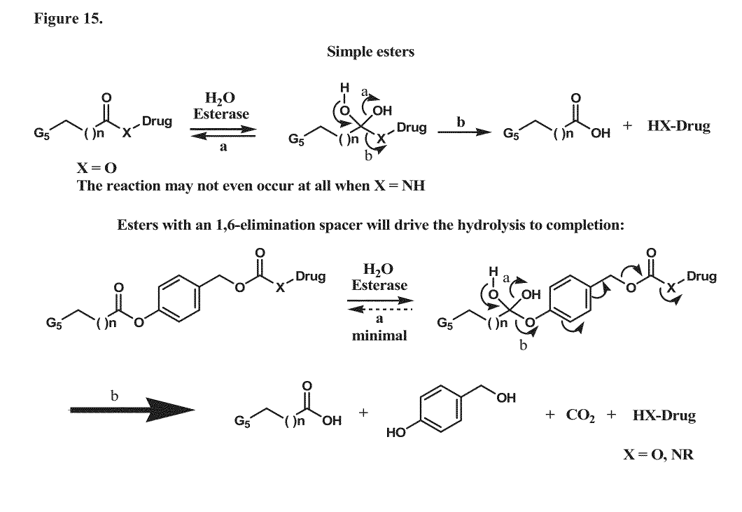

In some embodiments, a dendrimer conjugate of the present invention (e.g., a Baker-Huang PAMAM dendrimer conjugate) comprises a dendrimer conjugated to a linker (e.g., optionally conjugated to a trigger) that is conjugated to a therapeutic agent. In some embodiments, the dendrimer conjugate comprises a self-immolative connector between an ester bond (e.g., that is to be cleaved) and the therapeutic agent (e.g., thereby enhancing drug release). For example, although a mechanism is not necessary to practice the present invention and the present invention is not limited to any particular mechanism of action, in some embodiments, a dendrimer conjugate of the present invention comprising an ester linkage undergoes esterase catalyzed hydrolysis (e.g., as shown in FIG. 15 (e.g., G5 dendrimer comprising a self-degradable spacer and therapeutic agent)). Thus, in contrast to a dendrimer comprising a simple ester (e.g., a dendrimer in the top portion of FIG. 15 wherein therapeutic agent release may or may not occur, e.g., if x=NH), in some embodiments, the present invention provides a dendrimer conjugate comprising an elimination linker (e.g., a 1, 6, elimination linker/spacer as shown in the bottom portion of FIG. 15 (e.g., that permits complete hydrolysis of the linker (e.g., at a target site))).

The present invention is not limited by the type of linker configuration. In some embodiments, the linker is conjugated via a free amino group via an amide linkage (e.g., formed from an active ester (e.g., the N-hydroxysuccinimide ester)). In some embodiments, an ester linkage remains in the conjugate after conjugation. In some embodiments, linkage occurs through a lysine residue. In some embodiments, conjugation occurs through a short-acting, degradable linkage. The present invention is not limited by the type of degradable linkage utilized. Indeed, a variety of linkages are contemplated to be useful in the present invention including, but not limited to, physiologically cleavable linkages including ester, carbonate ester, carbamate, sulfate, phosphate, acyloxyalkyl ether, acetal, and ketal linkages. In some embodiments, a dendrimer conjugate comprises a cleavable linkage present in the linkage between the dendrimer and linker and/or targeting agent and/or therapeutic agent present therein (e.g., such that when cleaved, no portion of the linkage remains on the dendrimer). In some embodiments, a dendrimer conjugate comprises a cleavable linkage present in the linker itself (e.g., such that when cleaved, a small portion of the linkage remains on the dendrimer).

In some embodiments, conjugation between a dendrimer (e.g., terminal arm of a dendrimer) and a functional group or between functional groups is accomplished through use of a 1,3-dipolar cycloaddition reaction (“click chemistry”). ‘Click chemistry’ involves, for example, the coupling of two different moieties (e.g., a therapeutic agent and a functional group) (e.g., a first functional group and a second functional group) via a 1,3-dipolar cycloaddition reaction between an alkyne moiety (or equivalent thereof) on the surface of the first moeity and an azide moiety (e.g., present on a triazine composition) (or equivalent thereof) (or any active end group such as, for example, a primary amine end group, a hydroxyl end group, a carboxylic acid end group, a thiol end group, etc.) on the second moiety (see, e.g., U.S. Provisional Patent App. No. 61/140,480, herein incorporated by reference in its entirety. ‘Click’ chemistry is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932; each herein incorporated by reference in their entireties).

In some embodiments, conjugation between a dendrimer (e.g., terminal arm of a dendrimer) and a functional group or between functional groups is accomplished through use of copper-free click chemistry. “Copper-free click chemistry” involves, for example, the coupling of two different moieties (e.g., a therapeutic agent and a functional group) (e.g., a first functional group and a second functional group) via a copper-free Huisgen 1,3-dipolar cycloaddition reaction between a cyclooctyne moeity (or equivalent thereof) on the surface of the first moeity and an azide moiety (or equivalent thereof) (or any active end group such as, for example, a primary amine end group, a hydroxyl end group, a carboxylic acid end group, a thiol end group, etc.) on the second moiety. ‘Copper-free click chemistry’ is an attractive coupling method because, for example, it can be performed with a wide variety of solvent conditions including aqueous environments. Moreover, copper-free click chemistry avoids copper related cytotoxicity issues found in Cu(I)-catalyzed alkyne azide 1,3-dipolar cycloaddition.

The present invention is not limited to particular cyclooctyne moieties (or equivalents thereof). In some embodiments, the cyclooctyne moiety comprises the following formula:

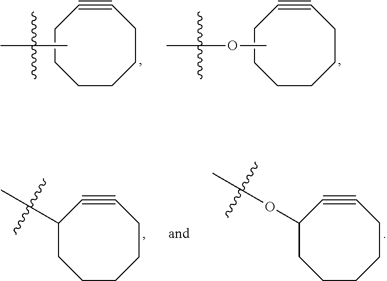

The present invention is not limited to a particular manner of conjugating the azide moieties (or equivalents thereof) and/or the cyclooctyne moieities (or equivalents thereof) wither either a dendrimer structure and/or a functional group. In some embodiments, the azide moieties (or equivalents thereof) and/or the cyclooctyne moieities (or equivalents thereof) are conjugated with either a dendrimer and/or a functional group via a primary amine end group, a hydroxyl end group, a carboxylic acid end group, a thiol end group, etc.). In certain embodiments, the dendrimer platform is linked with a cyclooctyne ligand and the functional group (e.g., methotrexate) linked with an azide moiety (e.g., as opposed to the dendrimer platform linked with the azide moiety and the functional group linked with the cyclooctyne ligand) (e.g., so as to increase solvent flexibility) (e.g., so as to reduce purification difficulty). As such, in preferred embodiments, the dendrimer platform is linked with the cyclooctyne ligand and the azide moiety linked with the functional group.

In some embodiments, conjugation between a dendrimer (e.g., a terminal arm of a dendrimer) and a functional ligand is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino) pyridine) (see, e.g., U.S. Provisional Patent App. No. 61/226,993, herein incorporated by reference in its entirety).

Functionalized nanoparticles (e.g., dendrimers) often contain moieties (including but not limited to ligands, functional ligands, conjugates, therapeutic agents, targeting agents, imaging agents, fluorophores) that are conjugated to the periphery. Such moieties may for example be conjugated to one or more dendrimer branch termini. Classical multi-step conjugation strategies used during the synthesis of functionalized dendrimers generate a stochastic distribution of products with differing numbers of ligands attached per dendrimer molecule, thereby creating a population of dendrimers with a wide distribution in the numbers of ligands attached. The low structural uniformity of such dendrimer populations negatively affects properties such as therapeutic potency, pharmacokinetics, or effectiveness for multivalent targeting. Difficulties in quantifying and resolving such populations to yield samples with sufficient structural uniformity can pose challenges. However, in some embodiments, use of separation methods (e.g., reverse phase chromatography) customized for optimal separation of dendrimer populations in conjunction with peak fitting analysis methods allows isolation and identification of subpopulations of functionalized dendrimers with high structural uniformity (see, e.g., U.S. Provisional Pat. App. No. 61/237,172; herein incorporated by reference in its entirety). In certain embodiments, such methods and systems provide a dendrimer product made by the process comprising: a) conjugation of at least one ligand type to a dendrimer to yield a population of ligand-conjugated dendrimers; b) separation of the population of ligand-conjugated dendrimers with reverse phase HPLC to result in subpopulations of ligand-conjugated dendrimers indicated by a chromatographic trace; and c) application of peak fitting analysis to the chromatographic trace to identify subpopulations of ligand-conjugated dendrimers wherein the structural uniformity of ligand conjugates per molecule of dendrimer within said subpopulation is, e.g., approximately 80% or more.

In some embodiments, the present invention also provides a kit comprising a composition comprising dendrimer conjugate comprising a linker and/or trigger and a therapeutic agent. In some embodiments, the kit comprises a fluorescent agent or bioluminescent agent.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, high performance liquid chromatography, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for applications and in vivo usage.

Dendrimer-antibody conjugates for use in in vitro diagnostic applications have previously been demonstrated (See, e.g., Singh et al., Clin. Chem., 40:1845 (1994)), for the production of dendrimer-chelant-antibody constructs, and for the development of boronated dendrimer-antibody conjugates (for neutron capture therapy); each of these latter compounds may be used as a cancer therapeutic (See, e.g., Wu et al., Bioorg. Med. Chem. Lett., 4:449 (1994); Wiener et al., Magn. Reson. Med. 31:1 (1994); Barth et al., Bioconjugate Chem. 5:58 (1994); and Barth et al.).

Some of these conjugates have also been employed in the magnetic resonance imaging of tumors (See, e.g., Wu et al., (1994) and Wiener et al., (1994), supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (See, e.g., Duncan and Malik, Control Rel. Bioact. Mater. 23:105 (1996)).

Dendrimers have also been conjugated to fluorochromes or molecular beacons and shown to enter cells. They can then be detected within the cell in a manner compatible with sensing apparatus for evaluation of physiologic changes within cells (See, e.g., Baker et al., Anal. Chem. 69:990 (1997)). Finally, dendrimers have been constructed as differentiated block copolymers where the outer portions of the molecule may be digested with either enzyme or light-induced catalysis (See, e.g., Urdea and Hom, Science 261:534 (1993)). This allows the controlled degradation of the polymer to release therapeutics at the disease site and provides a mechanism for an external trigger to release the therapeutic agents.

The dendrimers (e.g., Baker-Huang PAMAM dendrimers) may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the dendrimer population and are important in the quality control of dendrimer production for eventual use in in vivo applications. Most importantly, extensive work has been performed with dendrimers showing no evidence of toxicity when administered intravenously (Roberts et al., J. Biomed. Mater. Res., 30:53 (1996) and Boume et al., J. Magnetic Resonance Imaging, 6:305 (1996)).

An attractive feature of the present invention is that the therapeutic compositions may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

In some embodiments, the therapeutic complexes of the present invention comprise a photodynamic compound and a targeting agent that is administred to a patient. In some embodiments, the targeting agent is then allowed a period of time to bind the “target” cell (e.g. about 1 minute to 24 hours) resulting in the formation of a target cell-target agent complex. In some embodiments, the therapeutic complexes comprising the targeting agent and photodynamic compound are then illuminated (e.g., with a red laser, incandescent lamp, X-rays, or filtered sunlight). In some embodiments, the light is aimed at the jugular vein or some other superficial blood or lymphatic vessel. In some embodiments, the singlet oxygen and free radicals diffuse from the photodynamic compound to the target cell (e.g. cancer cell or pathogen) causing its destruction.

Where clinical applications are contemplated, in some embodiments of the present invention, the dendrimer conjugates are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In preferred embodiments, the dendrimer conjugates are used in conjunction with appropriate salts and buffers to render delivery of the compositions in a stable manner to allow for uptake by target cells. Buffers also are employed when the dendrimer conjugates are introduced into a patient. Aqueous compositions comprise an effective amount of the dendrimer conjugates to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active dendrimer conjugates may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, dendrimer conjugates are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The dendrimer conjugates also may be formulated as inhalants for the treatment of lung cancer and such like.

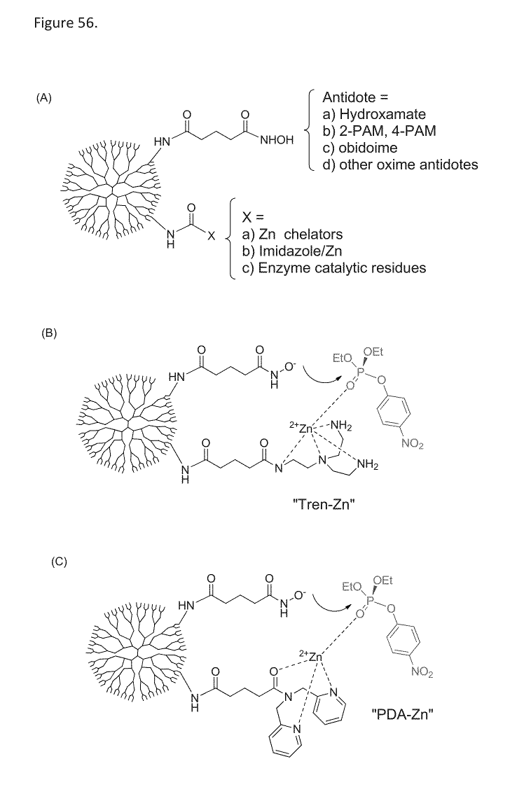

In certain embodiments, the present invention provides a dendrimer conjugate comprising both oxime-based therapeutic molecules and auxiliary groups such as metal chelators (FIG. 56). The therapeutic benefit for attaching such auxiliary groups is illustrated in the proposed mechanism of OP (PDX) hydrolysis where the auxiliary group plays a significant role by facilitating the catalytic reaction mediated by the oxime or hydroxamate of the attached drug molecule. Examples of those metal chelating auxiliary groups are based, for example, on the amine, imidazole, pyridine, and carboxylate groups, and include Tren, PDA, and PCA, but not limited here. Metal ions to be chelated include, but are not limited to, zinc, copper and other physiologic cations that are able to chelate to the P═O of the OP molecule and to make the phosphorous bond more susceptible for the hydrolytic cleavage.

### Examples

The following examples are provided in order to demonstrate and further illustrate certain preferred embodiments and aspects of the present invention and are not to be construed as limiting the scope thereof.

### Example I

Previous experiments involving dendrimer related technologies are located in U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/562,767, 61/568,521, 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/054202, PCT/US2010/050893, PCT/US2010/050893, PCT/U52010/042556, PCT/US2001/015204, PCT/US2005/030278, PCT/US2009/069257, PCT/US2009/036992, PCT/US2009/059071, PCT/US2007/015976, and PCT/US2008/061023, each herein incorporated by reference in their entireties.

### Example II

This example describes the synthesis of drug-conjugated dendrimers.

Materials and General Methods

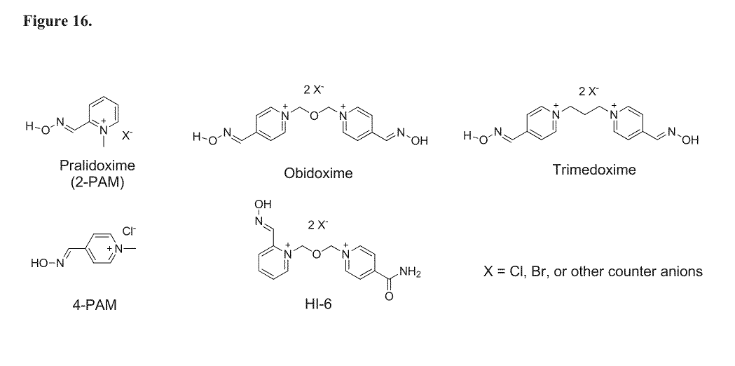

Pralidoxime chloride (2-PAM), and obidoxime chloride were purchased from Sigma-Aldrich, and used as received (FIG. 16). All solvents and reagents were purchased from commercial suppliers, and used without further purification. The PAMAM derimers studied here are based on ethylenediamine-cored a fifth generation (G5) PAMAM dendrimer (G5-NH₂, Dendritech, Inc). The commercial G5-NH₂ was provided in the methanolic solution, and purified prior to use by the process comprised of concentration in vacuo, and extensive dialysis of the residue against water (MWCO˜10,000) for 2 days. The number of primary amine groups per dendrimer molecule in G5-NH₂ was determined to be 114 on a mean basis by the potentiometric titration method as described elsewhere (see, e.g., Majoros, I. J.; J. Med. Chem. 2005, 48, 5892-99; herein incorporated by reference in its entirety). G5-(Glutaric Acid)₁₀₈ (G5-GA) was prepared by reacting G5-(NH₂)₁₁₄ with excess amount of glutaric anhydride in MeOH at room temperature (rt) as reported elsewhere (see, e.g., Choi, S. K.; Chem. Commun. 2010, 46, 2632-34; herein incorporated by reference in its entirety).

Characterization of compounds was typically carried out by ¹H NMR spectroscopy, mass spectrometry, and UV/vis spectrometry. For the NMR measurement, each sample was dissolved in a deuterated solvent (CD₃OD, D₂O), and the spectrum was acquired with a Varian nuclear magnetic resonance spectrometer at 500 MHz under a standard observation condition. The molecular weights (MW) for the G5 PAMAM dendrimer and its drug conjugates were measured by matrix assisted laser desorption ionization-time of flight (MALDI TOF) with a Waters TOfsPec-2E spectrometer as described elsewhere. The spectrometer was mass calibrated with BSA in sinapinic acid, and data was acquired and processed using Mass Lynx 3.5 software. UV-vis absorption spectra were recorded on a Perkin Elmer Lambda 20 spectrophotometer.

The purity of each dendrimer conjugate was determined by HPLC which was carried out on a Waters Acquity Peptide Mapping System equipped with a Waters photodiode array detector (an UPLC system). Each sample solution was run on a C4 BEH column (150×2.1 mm, 300 Å) connected to Waters Vanguard column. Elution of the conjugate was performed in a linear gradient beginning with 98:2 (v/v) water/acetonitrile (with trifluoroacetic acid at 0.14 wt % in each eluent) at a flow rate of 1 mL/min.

Gel permeation chromatography (GPC) experiments were performed to measure molecular weights and polydispersity index (PDI) of PAMAM G5 dendrimers. The GPC experiment was performed on an Alliance Waters 2695 separation module equipped with a 2487 dual wavelength UV absorbance detector (Waters Corporation), a Wyatt HELEOS Multi Angle Laser Light Scattering (MALLS) detector, and an Optilab rEX differential refractometer (Wyatt Technology Corporation). The isocratic mobile phase was 0.1 M citric acid and 0.025 wt % sodium azide, pH 2.74, at a flow rate of 1 mL/min. The sample concentration was 10 mg/5 mL. The weight average molecular weight (M_{w}) was determined by GPC data and the number average molecular weight (Mₙ) was calculated with Astra 5.3.14 software (Wyatt Technology Corporation) based on the molecular weight distribution. A polydispersity index (PDI=M_{w}/Mₙ) value determined for the purified G5 PAMAM dendrimer (G5-NH₂) is 1.010.

Representative Examples for the Synthesis of G5 PAMAM Dendrimers Conjugated with hydroxamate

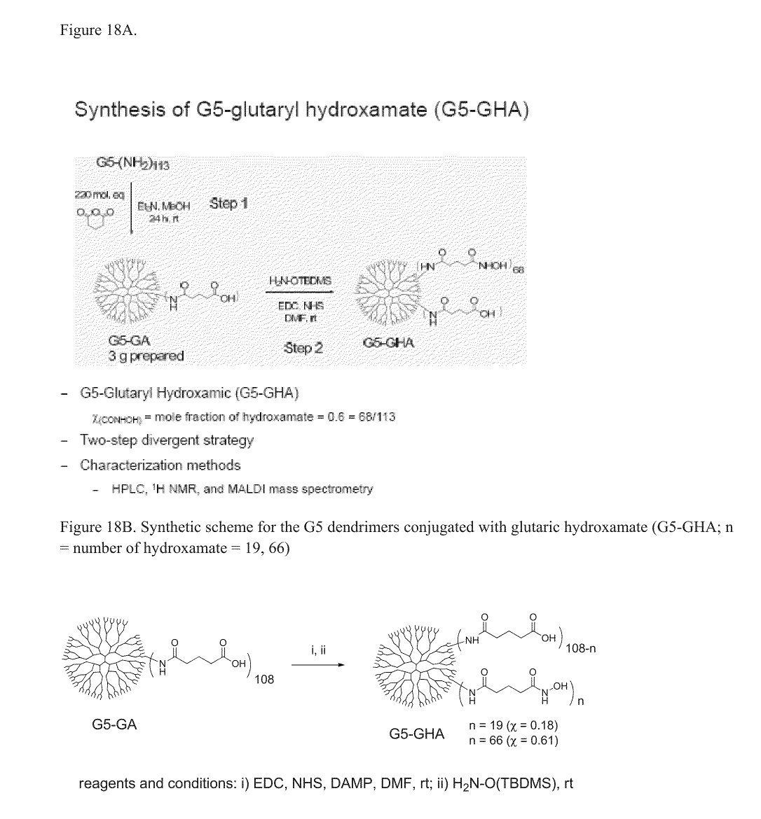

G5-GHA (n=66; FIG. 18B):

FIG. 18A shows a general synthesis scheme for G5-glutaryl hydroxamate (G5-GHA).

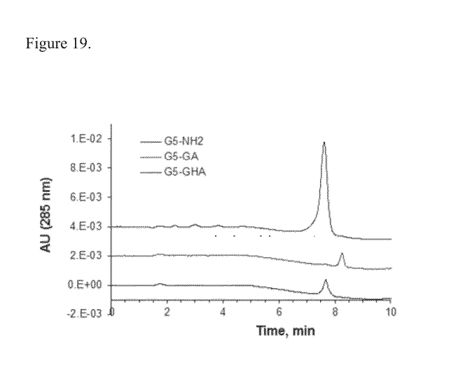

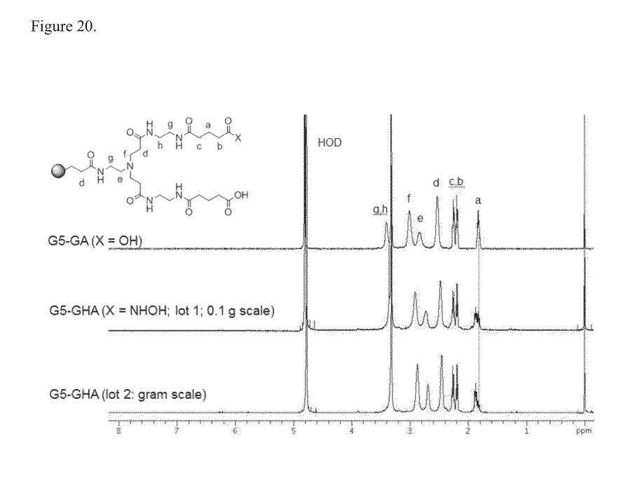

To the G5-GA dendrimer (MALDI MW=40,200 g/mol; 157 mg) suspended in anhydrous DMF (35 mL) was added N-hydroxysuccinimide (NHS, 97 mg), 4-dimethylaminopyridine (MDAP, 103 mg), and then 1-ethyl-3-(3-dimethylaminopropyl) carbodiimide hydrochloride (EDC, 105 mg). The reaction mixture was stirred at rt. After stirring for 24 hour (hr) when the solution became homogenous, O-(t-butyldimethylsilyl)protected hydroxylamine (124 mg) was added to the mixture: [H₂N—O(TBDMS)]/[glutaric acid]=2. The final reaction mixture was stirred at rt for additional 48 hr. To terminate the reaction, water (5 mL) was added to the mixture and concentrated in vacuo, yielding colorless residue. As an illustration for the typical purification process, the reaction mixture was concentrated in vacuo, and the residue was dissolved in 10 mL of phosphate buffered saline (PBS without Ca²⁺ and Mg²⁺; 10 mL), pH 7.4). The solution was loaded into a membrane dialysis bag (Spectrum® Labs, Inc.; MWCO 10 kDa), and dialyzed against PBS (2×2 L), and deionized water (3×2 L) over 3 days. The aqueous solution was collected and freeze-dried to afford the G5-GHA as white solid (125 mg). The purity of the dendrimer was analyzed by the HPLC method (FIG. 19): tᵣ=7.85 min; purity ≧99%. The number of hydroxamates attached to the peripheral branches of the dendrimer was estimated on a mean basis by the integration method of ¹H NMR spectral peaks (FIG. 20). The peaks used for the analysis come from CH₂ protons located in the middle of the glutaric acid spacer (assigned as protons a). Ratio of the integration area for CH₂ peaks between the glutaryl hydroxamate (Int_{C(═O)NHOH}) and the unmodified glutaric acid (Int_{C(═O)OH}) is used to determine the number (n) of the hydroxamate: Int_{C(═O)NHOH}/Int_{C(═O)OH}=n/(108-n). ¹H NMR (500 MHz, D₂O): δ 3.35 (s), 2.85 (s), 2.70 (s), 2.45 (s), 2.25 (m), 22.0 (m), 1.85 (m), 1.80 (m) ppm.

G5-GHA (n=19; FIG. 18B):

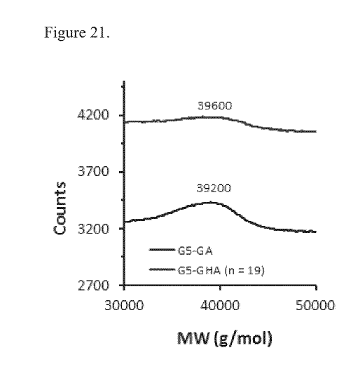

The other dendrimer hydroxamate that contains the lower number of glutaryl hydroxamates was prepared in the similar manner but by using of smaller amount of the hydroxylamine reactant ([H₂N-OTBDMS]/[glutaric acid]=0.3). The conjugation reaction starting with 123 mg of G5-GA led to isolation of the dendrimer hydroxamate (135 mg) as white fluffy solid. MALDI TOF mass spectrometry (m/z, gmol⁻¹): 39600 (FIG. 21). ¹H NMR (500 MHz, D₂O): δ 3.35 (s), 2.85 (s), 2.70 (s), 2.45 (s), 2.25 (m), 22.0 (m), 1.85 (m), 1.80 (m) ppm.

G5-Cyclopentane Fused Glutaric Hydroxamate (G5-GHAcp):

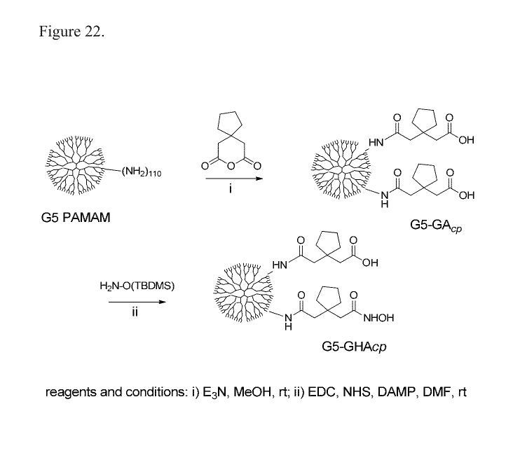

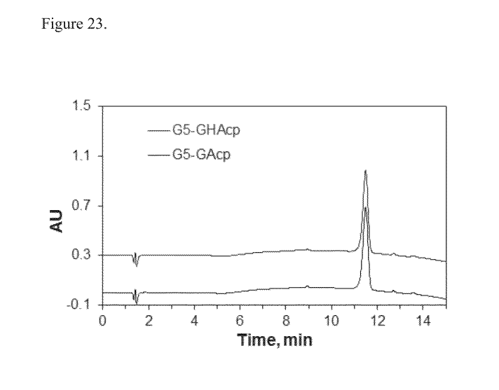

Step 1 (FIG. 22): To a solution of G5 PAMAM dendrimer (50 mg) dissolved in MeOH (15 mL) was added triethylamine (0.145 mL) and cyclopentane-fused glutaric anhydride (70 mg, 2 molar eq. to each NH₂ branch). The mixture was stirred at rt for 12 hr, and concentrated in vacuo. After dissolving the residue in PBS (10 mL), the solution was loaded into a membrane dialysis tubing (MWCO 10 kDa), and dialyzed extensively against PBS (1×2 L), and deionized water (3×4 L) over 3 days. The solution in the tubing was collected and lyophilized, yielding G5-GAcp as colorless foam (85 mg). The purity of the dendrimer was determined by the HPLC method (FIG. 23): tᵣ=11.4 min; purity ≧99%. The molecular weight of the dendrimer was characterized by measuring MALDI-TOF: m/z=45400. ¹H NMR (500 MHz, D₂O): δ 3.45 (broad s), 3.35 (s), 3.20-3.15 (broad s), 3.15-2.8 (broad m), 2.60 (broad s), 2.35 (s), 2.25 (m), 1.65 (m), 1.55 (m) ppm.

Step 2 (FIG. 22):

To the G5-GAcp dendrimer (35 mg) suspended in anhydrous DMF (10 mL) was added N-hydroxysuccinimide (NHS, 20 mg), 4-dimethylaminopyridine (MDAP, 21 mg), and then 1-ethyl-3-(3-dimethylaminopropyl) carbodiimide hydrochloride (EDC, 25 mg). The reaction mixture was stirred at rt, while the solution became homogenous. After stirring for 24 hr, O-(t-butyldimethylsilyl)protected hydroxylamine (19 mg) was added to the mixture: [H₂N—O(TBDMS)]/[dendrimer]=167. The final reaction mixture was stirred at rt for additional 24 hr. The reaction was terminated by adding water (5 mL) and the mixture was concentrated in vacuo, yielding colorless residue. The residue was dissolved in 5 mL of PBS (pH 7.4), and loaded into a membrane dialysis bag (MWCO 10 kDa). After the dialysis against PBS (2×2 L), and deionized water (3×2 L) over 3 days, the aqueous solution was collected and freeze-dried to afford the G5-GHAcp as white solid (29 mg). The purity of the dendrimer was analyzed by the HPLC method (FIG. 23): tᵣ=11.5 min; purity ≧99%. ¹H NMR (500 MHz, D₂O): δ 3.35 (broad s), 2.9 (broad m), 2.7 (broad s), 2.45 (broad s), 2.4-2.3 (m), 2.25 (m), 1.6 (braod m), 1.6-1.5 (m) ppm.

Synthesis of G5 Dendrimer Conjugated with Pyridiniumaldoxime (G5-PAM)

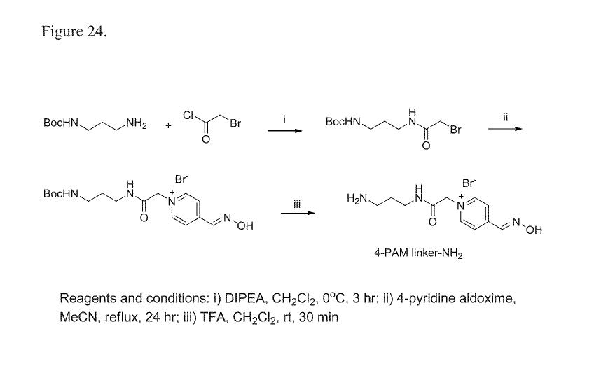

Synthesis of 4-PAM Linker-NH₂ (FIG. 24):

Step i) [(Bromoacetyl)amino]propyl]-carbamic acid 1,1-dimethylethyl ester was prepared as described elsewhere (see, e.g., Arimoto, M.; J. antibiot 1986, 39, 1243-56; Choi, S. K.; Bioorg. Med. Chem. 2012, 20, 1281-90; each herein incorporated by reference in its entirety). To a cold solution of 3-(N-tert-butoxycarbonylamino)propylamine (1.1 g, 6.31 mmol) in CHCl₃ (50 mL) cooled with an ice bath was added N,N-diisopropylethylamine (1.1 mL, 2.03 mmol), and bromoacetyl chloride (526 μL, 6.32 mmol) as neat liquid. The reaction mixture was stirred at 5° C. for 3 h under nitrogen atmosphere. The mixture was diluted with dichloromethane (200 mL), and it was washed with 1M H₃PO₄ (50 mL) solution, and a saturated sodium bicarbonate solution (50 mL). The organic layer was dried over Na₂SO₄, and evaporated to dryness in vacuo yielding colorless syrup. The product was gradually solidified to white crystals (1.9 g). It was used immediately for the next step withought further purification. R_{f} (50% EtOAc/hexane)=0.40.

Step ii) To a solution of N-bromoacetyl-1,3-diaminopropane (6.3 mmol) in acetonitrile (70 mL), prepared fresh in the earlier step, was added 4-pyridinealdoxime (0.848 g, 6.9 mmol). The mixture was refluxed under the nitrogen atmosphere for 24 hr, and concentrated to dryness in vacuo, yielding pale brown residue. This crude material was rinsed with a copious volume of ethyl acetate (50 mL), and the solid material was dried to afford the coupled product (N-Boc proteted 4-PAM linker-NHBoc) as pale brown solid (1.25 g, 48%). HRMS (ESI): m/z calcd for C₁₆H₂₅N₄O₄ [M-Br] 337.1870, found 337.1871. ¹H NMR (500 MHz, CD₃OD): δ 8.84 (d), 8.54 (d), 8.35 (s), 8.26 (d), 8.11 (s), 7.63 (d), 5.41 (s, 2H), 3.36 (m, 2H), 3.13 (m, 2H), 1.72 (m), 1.45 (s, 9H) ppm.

Step iii) To a suspension of the N-Boc proteted 4-PAM linker (1.15 g, 2.76 mmol) in dichloromethane (10 mL) was added trifluoroacetic acid (10 mL). The solid material was solubilized immediately. It was stirred at rt for 30 min, and concentrated to approximately 5 mL in vacuo. The solution was slolwy titarted into the stirred solution of diethylether (100 mL). The product was precipitated and collected by decanting the supernatant. The solid material was rinsed with ether (50 mL), and dried. This material was dissolved in water (20 mL), and freeze-dried to afford the product as the pale brown solid. ¹H NMR (500 MHz, CD₃OD): δ 8.84 (d, 2H), 8.23 (d, 2H), 5.44 (s, 2H), 3.36 (m, 2H), 3.00 (m, 2H), 1.99 (m, 2H) ppm.

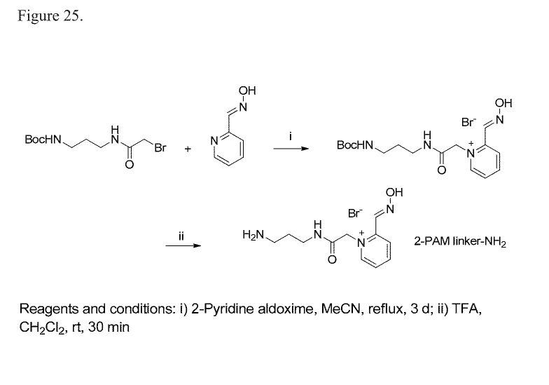

Synthesis of 2-PAM Linker-NH₂ (FIG. 25):

Step i) To a solution of N-bromoacetyl-1,3-diaminopropane (5.2 mmol) in acetonitrile (70 mL), prepared fresh in the earlier step, was added 2-pyridinealdoxime (0.694 g, 5.7 mmol). The mixture was refluxed under the nitrogen atmosphere for 3 d, and concentrated to dryness in vacuo, yielding pale brown residue. This crude material was suspended in ethyl acetate (50 mL), and collected by filtration. The solid material was then rinsed with acetone (50 mL), and dried to afford the desired product (N-Boc proteted 2-PAM linker-NHBoc) as pale brown solid (0.36 g, 17%). HRMS (ESI): m/z calcd for C₁₆H₂₅N₄O₄ [M-Br] 337.1870, found 337.1869. ¹H NMR (500 MHz, CD₃OD): δ 8.86 (d, 1H), 8.61 (t, 1H), 8.50-8.48 (m, 2H), 8.06 (t, 1H), 5.61 (s, 2H), 3.31 (m, 2H), 3.11 (t, 2H), 1.70 (m, 2H), 1.44 (s, 9H) ppm.

Step ii) To a suspension of the N-Boc proteted 2-PAM linker (0.237 g, 0.057 mmol) in dichloromethane (2 mL) was added trifluoroacetic acid (2 mL). The solid material was solubilized immediately. It was stirred at rt for 30 min, and concentrated to approximately ˜1 mL by using nitrogen flow. The solution was slowly titarted into the stirred solution of diethylether (50 mL). The product was precipitated and collected by spinning. The solid material was rinsed with ether (20 mL), and the hygroscopic solid was obtained. It was dissolved in water (5 mL), and freeze-dried to afford the product as the pale brown solid. HRMS (ESI): m/z calcd for C₁₁H₁₇N₄O₂ [M-Br] 237.1346, found 237.1347. ¹H NMR (500 MHz, D₂O): δ 8.80 (d, 1H), 8.65 (t, 1H), 8.52 (s, 1H), 8.41 (d, 1H), 8.09 (t, 1H), 5.67 (s, 2H), 3.40 (t, 2H), 3.03 (t, 2H), 1.93 (quin, 2H) ppm.

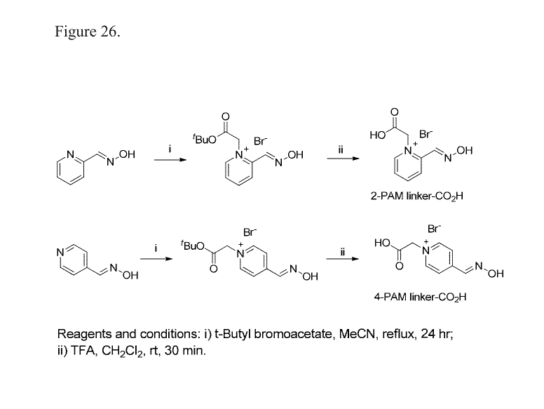

Synthesis of PAM Linker-CO₂H (FIG. 26):

2-PAM linker-CO₂H: To a solution of 2-pyridinealdoxime (1.0 g, 8.2 mmol) in acetonitrile (70 mL) was added t-butyl bromoacetate (2.4 g, 12.3 mmol). The mixture was refluxed under the nitrogen atmosphere for 24 hr, and evaporated to dryness in vacuo, yielding pale brown residue. This crude product was suspended in acetone (50 mL), collected and rinsed with acetone (50 mL). The product (t-butyl proteted 2-PAM linker-CO₂H) was obtained as pale brown solid (1.0 g, 39%). HRMS (ESI): m/z calcd for C₁₂HᵣN₂O₃ [M-Br] 237.1234, found 237.1233. ¹H NMR (500 MHz, D₂O): δ 8.82 (d, 1H), 8.66 (t, 1H), 8.57 (s, 1H), 8.39 (d, 1H), 8.11 (t, 1H), 5.65 (s, 2H), 1.34 (s, 9H) ppm. The t-butyl protecting group of the above product was removed by treatment with TFA as follows. To a suspension of the above product (0.7 g, 2.2 mmol) in dichloromethane (4 mL) was added trifluoroacetic acid (7 mL). The mixture was stirred at rt for 35 min, and concentrated to in vacuo, yielding brown oily residue. It was dissolved in acetonitrile (10 mL), and evaporated again to dryness. The oily material was dried under nitrogen flow, and slowly turned to the brown solid (0.703 g). HRMS (ESI): m/z calcd for C₈H₉N₂O₃ [M-Br] 181.0608, found 181.0603. ¹H NMR (500 MHz, D₂O): δ 8.78 (d, 1H), 8.60 (t, 1H), 8.59 (s, 1H), 8.38 (d, 1H), 8.05 (t, 1H), 5.46 (s, 2H) ppm.

4-PAM linker-CO₂H:

To a solution of 4-pyridinealdoxime (1.0 g, 8.2 mmol) in acetonitrile (70 mL) was added t-butyl bromoacetate (2.4 g, 12.3 mmol). The mixture was refluxed under the nitrogen atmosphere for 24 hr, and evaporated to dryness in vacuo, yielding pale brown residue. This crude product was suspended in acetone (50 mL), collected and rinsed with acetone (50 mL). The product (t-butyl proteted 2-PAM linker-CO₂H) was obtained as pale brown solid (2.49 g, 96%). HRMS (ESI): m/z calcd for C₁₁H₁₇N₄O₂ [M-Br] 237.1234, found 237.1237. The t-butyl protecting group of the above product was removed by treatment with TFA as follows. To a suspension of the above product (1.5 g, 4.7 mmol) in dichloromethane (10 mL) was added trifluoroacetic acid (15 mL). The mixture was stirred at rt for 30 min, and concentrated to in vacuo, yielding brown residue. It was suspended in acetone (50 mL), collected by filtration, and rinsed with acetone (50 mL). HRMS (ESI): m/z calcd for C₈H₉N₂O₃ [M-Br] 181.0608, found 181.0607. ¹H NMR (500 MHz, D₂O): δ 8.73 (d, 2H), 8.40 (s, 1H), 8.22 (d, 2H), 5.21 (s, 2H) ppm.

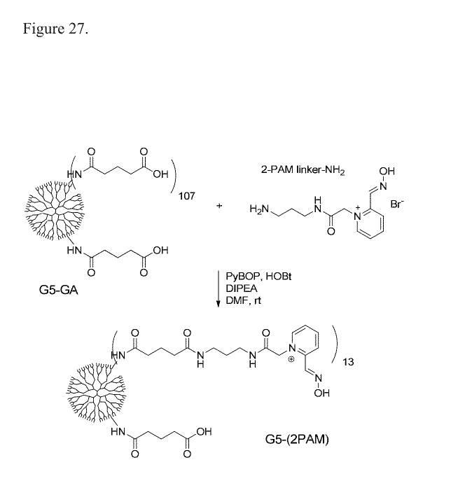

Synthesis of G5 Dendrimer Conjugated with 2-PAM (G5-2PAM; FIG. 27):

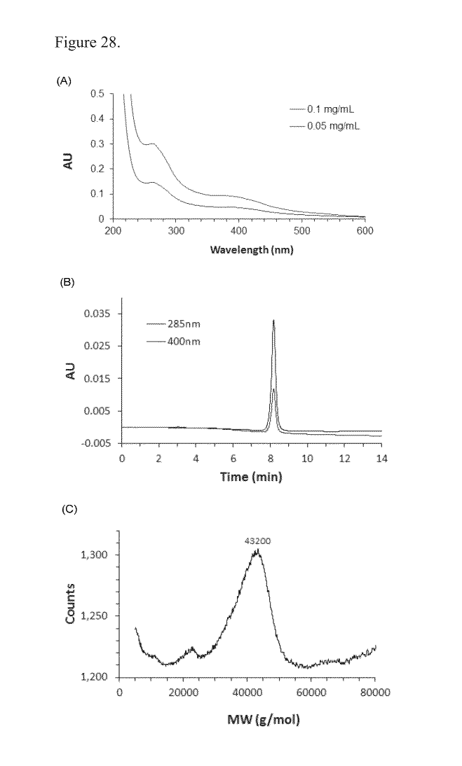

To the G5-GA dendrimer (MW=40,200 g/mol; 82 mg) suspended in anhydrous DMF (15 mL) was added N-hydroxysuccinimide (NHS, 28 mg), 4-dimethylaminopyridine (MDAP, 55 mg), and then 1-ethyl-3-(3-dimethylaminopropyl) carbodiimide hydrochloride (EDC, 63 mg). The mixture became homogenous while it was stirred at rt for 24 hr. Then a solution of 2-PAM linker-NH₂ (TFA salt; 35 mg, 40 molar eq to the dendrimer) dissolved in DMF (1 mL) containing triethylamine (0.057 mL) was added to the activated dendrimer solution. The final reaction mixture was stirred at rt for additional 12 hr. The reaction was terminated by adding water (5 mL), and the mixture was concentrated to approximately 2 mL in vacuo. The residue was dissolved in 10 mL of PBS (10 mL, pH 7.4), and loaded into a membrane dialysis bag (MWCO 10 kDa). The solution inside the tubing was dialyzed against PBS (2×2 L), and deionized water (3×2 L) over 3 days. The aqueous content was freeze-dried to afford the G5-(2PAM) as pale brown solid (74 mg). The dendrimer was characterized by a number of analytical methods as summarized in FIG. 28. HPLC analysis: tᵣ=8.19 min; purity ≧99%. MALDI-TOF: m/z (gmol⁻¹)=43200. UV/vis spectroscopy (PBS, pH 7.4): λₘₐₓ=390, 270 nm. ¹H NMR (500 MHz, D₂O): δ 8.9-7.9 (low intensity, multiple peaks), 7.8-7.4 (low intensity, multiple peaks), 5.7 (low intensity, broad), 3.4-3.2 (broad m), 3.0-2.8 (broad m), 2.75 (m), 2.6-2.4 (broad m), 2.3 (m), 2.2 (m), 1.8 (m) ppm. The number (n) of 2-PAM molecules attached to the dendrimer was estimated on a mean basis (n=13) from the analysis of UV/vis, ¹H NMR, and MALDI-TOF mass spectroscopic data.

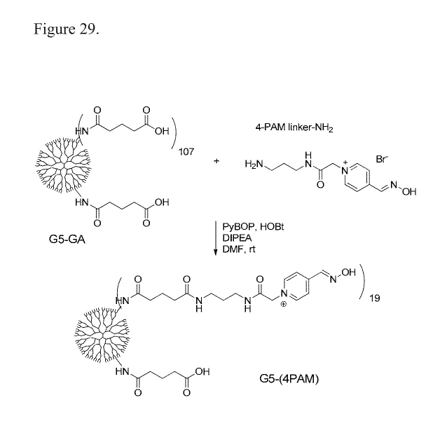

Synthesis of G5 dendrimer conjugated with 4-PAM (G5-(4PAM)ₙ₌₁₉ (FIG. 29):

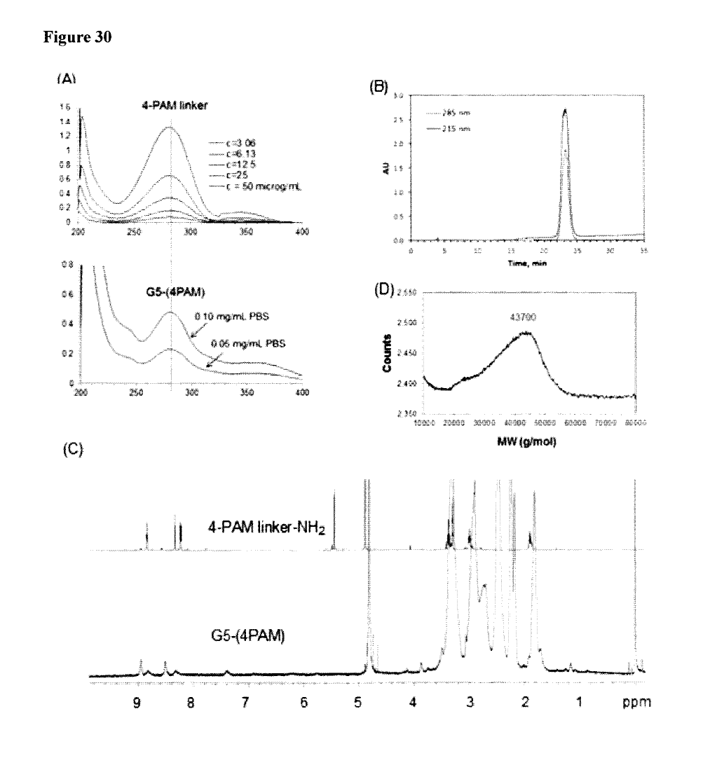

To the G5-GA dendrimer (MW=40,200 g/mol; 82 mg) suspended in anhydrous DMF (15 mL) was added N-hydroxysuccinimide (NHS, 28 mg), 4-dimethylaminopyridine (MDAP, 50 mg), and then 1-ethyl-3-(3-dimethylaminopropyl) carbodiimide hydrochloride (EDC, 63 mg). The mixture was stirred at rt and it became homogenous. After stirring for 12 hr, a solution of 4-PAM linker-NH₂ (TFA salt; 26 mg, 30 molar eq to the dendrimer) dissolved in DMF (1 mL) that contained triethylamine (0.040 mL) was added to the activated dendrimer solution. The final reaction mixture was stirred at rt for additional 18 hr. The reaction was terminated by adding water (5 mL), and the mixture was concentrated to approximately 2 mL in vacuo. The residue was dissolved in 10 mL of PBS (pH 7.4), and loaded into the membrane dialysis tubing (MWCO 10 kDa). The solution inside the tubing was dialyzed against PBS (2×2 L), and deionized water (3×2 L) over 3 days. The aqueous content was freeze-dried to afford the G5-(4PAM) as pale brown solid (64 mg). The dendrimer was characterized by a number of analytical methods as summarized in FIG. 30. HPLC analysis: tᵣ=23.1 min; purity ≧99%. MALDI-TOF: m/z (gmol⁻¹)=43700. UV/vis spectroscopy (PBS, pH 7.4): λₘₐₓ=360, 280 nm. ¹H NMR (500 MHz, D₂O): δ 9.0 (broad s), 8.8 (broad), 8.7 (broad s), 8.35 (broad), 7.4 (broad), 3.5-3.1 (broad m), 3.0-2.7 (broad m), 2.6-2.4 (broad m), 2.3-2.2 (broad m), 1.9-1.6 (broad m) ppm. The number (n) of 4-PAM molecules attached to the dendrimer was estimated on a mean basis (n=19) from the analysis of UV/vis, ¹H NMR, and MALDI-TOF mass spectroscopic data.

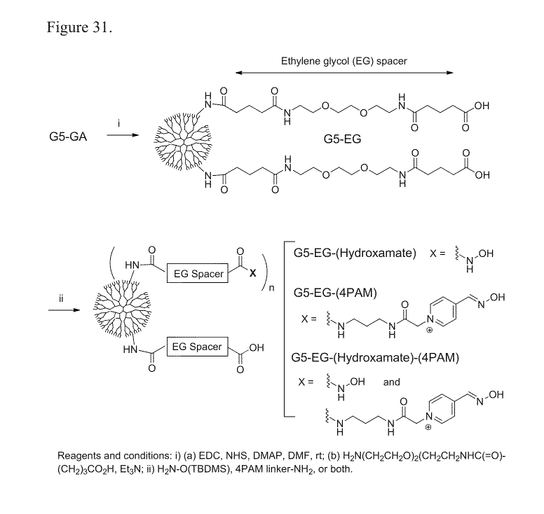

Synthesis of G5 Dendrimer Drug Conjugates, Each Linked with Hydroxamate and/or 4-PAM Via Extended Ethylene Glycol (EG) Spacer (G5-EG (FIG. 31)):

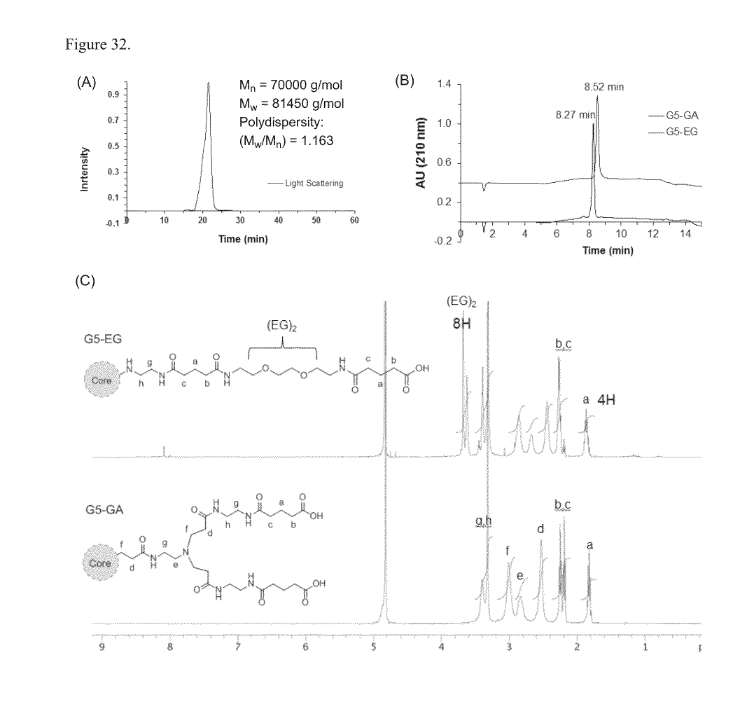

To the G5-GA dendrimer (MW=40,200 g/mol; 1.0 g, 0.25 mmol) suspended in anhydrous DMF (100 mL) was added N-hydroxysuccinimide (NHS, 515 mg), 4-dimethylaminopyridine (MDAP, 546 mg), and then 1-ethyl-3-(3-dimethylaminopropyl) carbodiimide hydrochloride (EDC, 618 mg, 1.2 mol eq to glutaric acid). The mixture was stirred at rt for 24 hr. Meantime, the ethylene glycol (EG)-based long spacer, H₂N(CH₂CH₂O)₂CH₂CH₂NHC(═O)(CH₂)₃CO₂H (or, 5-[[2-[2-(2-aminoethoxyl)ethoxy]ethyl]amino]-5-oxo-pentanoic acid), was prepared in a separate flask as follows. To the DMF (50 mL) solution containing triethylamine (1.88 mL, 13.5 mmol) was added 3,6-dioxaoctane-1,8-diamine (1.0 g, 6.7 mmol), and then glutaric anhydride (0.785 g, 6.9 mmol) as the solution in DMF (5 mL). The reaction mixture was stirred at rt while it became heterogenous as the thick oily residue was precipitated. After stirring for 12 hr, the mixture was concentrated to approximately 15 mL in vacuo, and the crude product was used for the next step without further treatment. This EG-based spacer in the DMF solution (2.5 molar eq to each glutaric acid residue of the dendrimer) was added to the preactivated dendrimer solution prepared earlier above. The final reaction mixture was stirred at rt for additional 12 hr prior to the addition of glutaric anhydride (0.4 g) to cap unreacted primary amine molecules left in the reaction mixture. After stirring for 12 hr, the reaction was terminated by adding water (20 mL), and the mixture was concentrated in vacuo. The wet residue was dissolved in 50 mL of PBS (pH 7.4), and loaded into the membrane dialysis tubing (MWCO 10 kDa). The solution inside the tubing was dialyzed against PBS (2×4 L), and deionized water (3×4 L) over 3 days. The aqueous content was freeze-dried to afford the G5-EG as beige solid (1.23 g). The dendrimer was characterized by a number of analytical methods as summarized in FIG. 32. HPLC analysis: tᵣ=8.52 min; purity ≧99%. GPC: M_{w}=81450 gmol⁻¹; Mₙ, =70000 gmol⁻¹; PDI=1.163. ¹H NMR (500 MHz, D₂O): δ 3.7 (s), 3.6 (s), 3.4-3.3 (m), 2.85 (broad s), 2.7 (broad s), 2.45 (broad s), 2.25 (m), 1.85 (m) ppm.

G5-EG-(Hydroxamate) (FIG. 31):

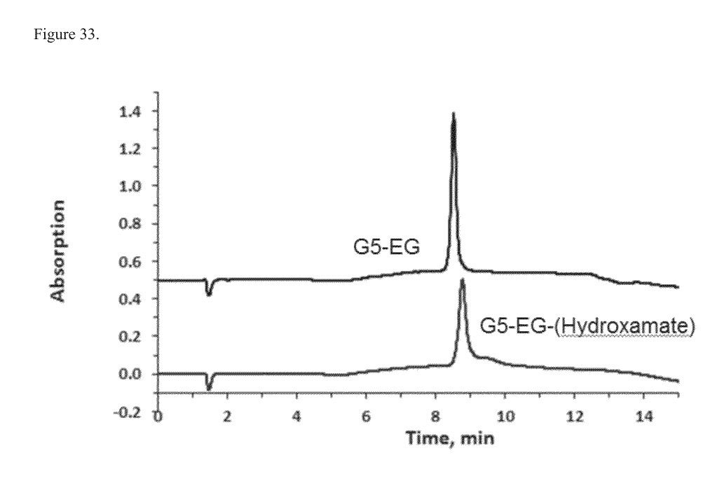

To the G5-EG dendrimer (mean MW=76000 gmol⁻¹; 54 mg) suspended in anhydrous DMF (10 mL) was added N-hydroxysuccinimide (NHS, 20 mg), 4-dimethylaminopyridine (MDAP, 42 mg), and then 1-ethyl-3-(3-dimethylaminopropyl) carbodiimide hydrochloride (EDC, 65 mg). The reaction mixture was stirred at rt. After stirring for 12 hr, O-(t-butyldimethylsilyl)protected hydroxylamine (23 mg) was added to the mixture: [H₂N—O(TBDMS)]/[carboxylic acid]≈2. The final reaction mixture was stirred at rt for additional 24 hr. To terminate the reaction, water (2 mL) was added to the mixture and concentrated in vacuo, yielding colorless residue. The residue was dissolved in 5 mL of PBS (pH 7.4), loaded into the membrane dialysis tubing (MWCO 10 kDa), and dialyzed against PBS (2×2 L), and deionized water (3×2 L) over 3 days. The aqueous solution was collected and freeze-dried to afford the G5-EG-(Hydroxamate) as white solid (30 mg). The purity of the dendrimer was analyzed by the HPLC method (FIG. 33): tᵣ=8.75 min; purity ≧99%.

G5-EG-(4PAM)ₙ₌₁₆ (FIG. 31):

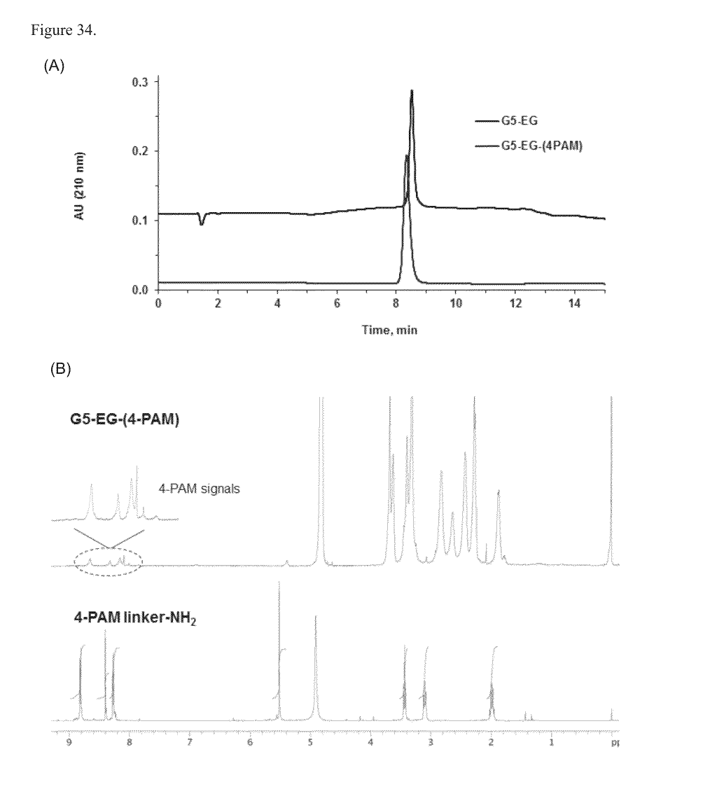

To the G5-EG dendrimer (mean MW=76000 gmol⁻¹; 100 mg) suspended in anhydrous DMF (10 mL) was added N-hydroxybenzotriazole (HOBt, 31 mg), diisopropylethylamine (DIPEA, 0.07 mL), and then PyBOP (104 mg). The reaction mixture was stirred at rt for 14 hr, and followed by the addition of 4-PAM linker-NH₂ (TFA salt; 99 mg) dissolved in DMF (1 mL) containing DIPEA (0.2 mL): [4-PAM linker]/[carboxylic acid]≈1.6. The final reaction mixture was stirred at rt for additional 24 hr. The reaction was terminated by adding water (2 mL) and the mixture was concentrated in vacuo, yielding colorless residue. The residue was dissolved in 5 mL of PBS (pH 7.4), loaded into the membrane dialysis tubing (MWCO 10 kDa), and dialyzed against PBS (2×2 L), and deionized water (3×2 L) over 3 days. The aqueous solution was collected and freeze-dried to afford the G5-EG-(4PAM) as white solid (87 mg). The purity of the dendrimer was determined by the HPLC method (FIG. 34): tᵣ=8.34 min; purity ≧99%. ¹H NMR (500 MHz, D₂O): δ 8.65 (broad s), 8.32 (broad s), 8.16 (broad s), 8.10 (s), 3.7-3.6 (m), 3.4-3.3 (m), 2.8 (broad s), 2.65 (broad s), 2.5 (broad s), 2.3 (broad s), 1.9 (m) ppm. The number (n) of 4-PAM molecules attached to the dendrimer was estimated on a mean basis (n=16) by the NMR integration method.

G5-EG-(Hydroxamate)-(4PAM):

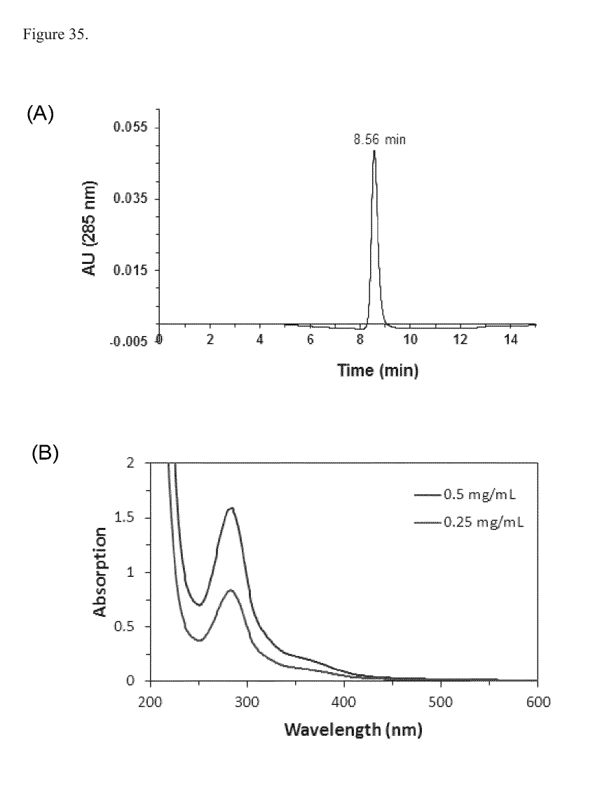

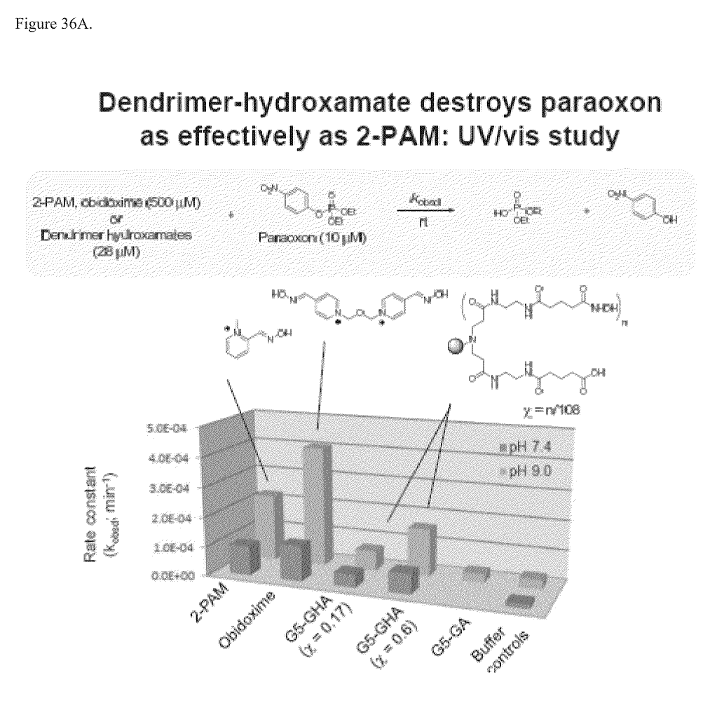

To the G5-EG dendrimer (mean MW=76000 gmol⁻¹; 1.3 g) suspended in anhydrous DMF (130 mL) was added N-hydroxybenzotriazole (HOBt, 421 mg), diisopropylethylamine (DIPEA, 0.96 mL), and then PyBOP (1.57 g). The reaction mixture was stirred at rt for 15 hr, and followed by the addition of 4-PAM linker-NH₂ (TFA salt; 432 mg) dissolved in DMF (1 mL) containing DIPEA (1.4 mL): [4-PAM linker]/[carboxylic acid]≈0.5. The final reaction mixture was stirred at rt for additional 24 hr when O-(t-butyldimethylsilyl)protected hydroxylamine (368 mg) was added to the mixture: [H₂N—O(TBDMS)]/[carboxylic acid]≈1.4. The reaction mixture was stirred further for 12 hr, and the reaction was terminated by adding water (20 mL). The mixture was concentrated in vacuo, yielding pale brown residue. The residue was dissolved in 50 mL of PBS (pH 7.4), loaded into the membrane dialysis tubing (MWCO 10 kDa), and dialyzed against PBS (2×4 L), and deionized water (3×4 L) over 3 days. The aqueous solution was collected and freeze-dried to afford the G5-EG-(Hydroxamate)-(4PAM) as pale brown solid (1.15 g). The purity of the dendrimer was determined by the HPLC method (FIG. 35): tᵣ=8.56 min; purity ≧99%. UV/vis spectroscopy (PBS, pH 7.4): λₘₐₓ=370, 280 nm. MALDI-TOF: m/z (gmol⁻¹)=83600. The number (n) of 4-PAM molecules attached to the dendrimer was estimated on a mean basis (n=15) from the analysis of UV/vis spectrometric, and ¹H NMR data.

### Example III

This example describes the determination of the OP scavenging activity of drug-conjugated dendrimers.

Colorimetric Assay

An in vitro reaction kinetics method was developed that enables determination of the chemical scavenging activity for the OP destruction. Paraoxon (PDX) was used as the model OP agent. The colorimetric method is based on UV/vis spectrometry, and allows evaluatation of the catalytic activity of oxime antidote molecules (eg., 2-PAM, obidoxime) or the dendrimer-drug conjugates for the PDX hydrolysis. This UV-based assay monitors the concentration of 4-nitrophenol, the byproduct of the PDX hydrolysis, at 400 nm which is strongly absorbed by 4-nitrophenol.

Experimental Details and Data Analysis

To a solution of 2-PAM (1 mL, 0.5 mM) prepared in PBS (pH 7.4) was added 0.01 mL of paraoxon (PDX; 1 mM in MeCN). This mixture was incubated at rt and its UV/vis spectra were taken at a series of time points: t=0 min (right after the mixing of the two compounds), 12 hr, 24 hr, 36 hr, 48 hr. This kinetic reaction was repeated in triplicate, each using three different sample solutions. The observed rate for the PDX hydrolysis was determined on the assumption that the reaction kinetics follows the pseudo-first order kinetics ([Oxime]/[PDX]≈500 for the early phase of the kinetics where less than 10% PDX hydrolysis occurs). By using the rate equation 1 (below), the observed rate constant (k_{obsd}) was determined from the slope. The half life (t_{1/2}) for the PDX hydrolysis is defined when [PDX]ₜ=0.5 [PDX]ₜ₌₀, and calculated as t_{1/2}=ln(2)/k_{obsd}.

ln[PDX]ₜ=−k_{obsd}×t+ln[PDX]ₜ₌₀ where[PDX]ₜ=[PDX]ₜ₌₀−[4-Nitrophenol]ₜ Eqn1.

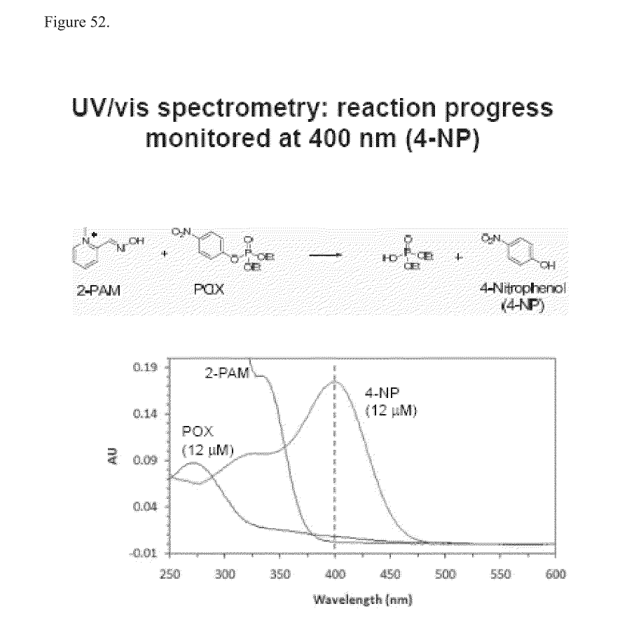

FIG. 52 shows the UV/vis spectrometry for 4-Nitrophenol production from 2-PAM and PDX.

2-PAM, obidoxime, and G5-GHA

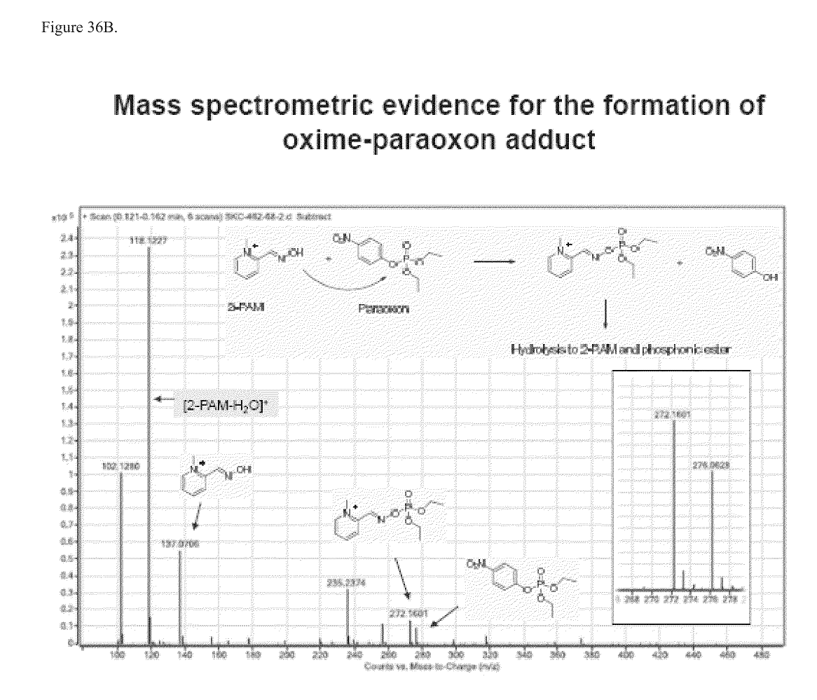

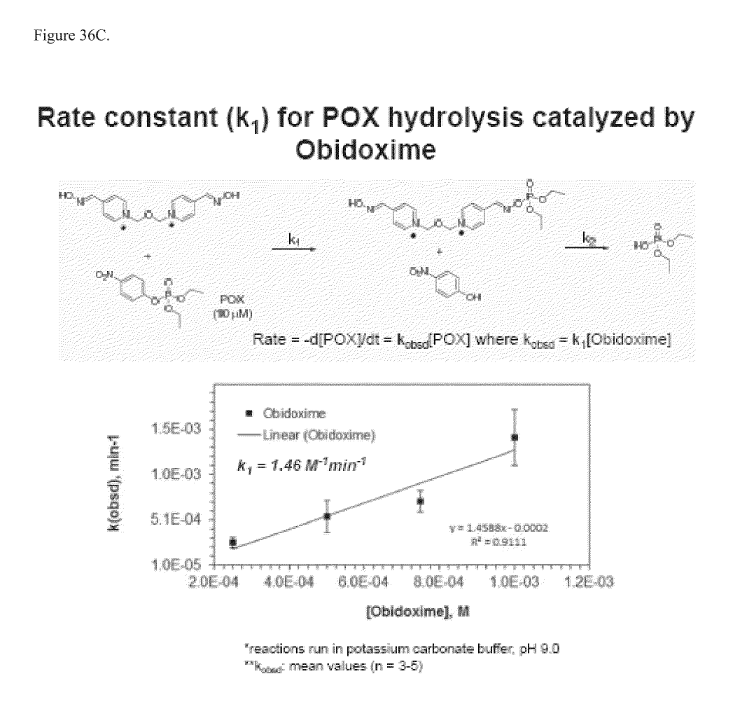

FIG. 36A shows the chemical scavenging activity pertinent to the PDX hydrolysis performed at two different pH conditions (pH 7.4, and 9.0). It also summarizes the catalytic activities determined for the two oxime drug molecules (2-PAM, obidoxime), and two hydroxamate-conjugated dendrimers (G5-GHA; not in complex with 2-PAM). The activity of the catalytic reaction is expressed in terms of an observed rate constant (k_{obsd}) such that higher rate constants refer to greater activities. At the physiological pH 7.4, both 2-PAM (k_{obsd}≈1.0×10⁻⁴), and obidoxime (k_{obsd}≈1.2×10⁻⁴) are catalytically active compared to the buffer control (k_{obsd}<1.0×10⁻⁵). Both of the G5-GHA conjugates showed significant activities though tested at the lower concentration (n=19: k_{obsd}≈4.5×10⁻⁵; n=66: k_{obsd}≈6.9×10⁻⁵). The scavenging activity by the drugs and the dendrimer conjugates are greater generally at pH 9. This pH dependency is consistent with the mechanism of drug action in which the oxime or hydroxamate exists as the more reactive deprotonated form at the alkaline condition.

FIG. 36B displays mass spectrometric evidence for the formation of oxime-paraoxon adduct.

FIG. 36C shows the rate constant (k₁) for PDX hydrolysis catalyzed by obidoxime.

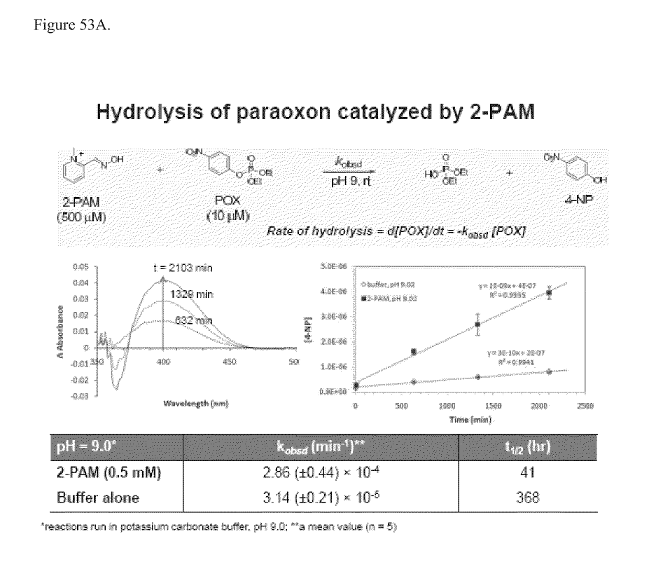

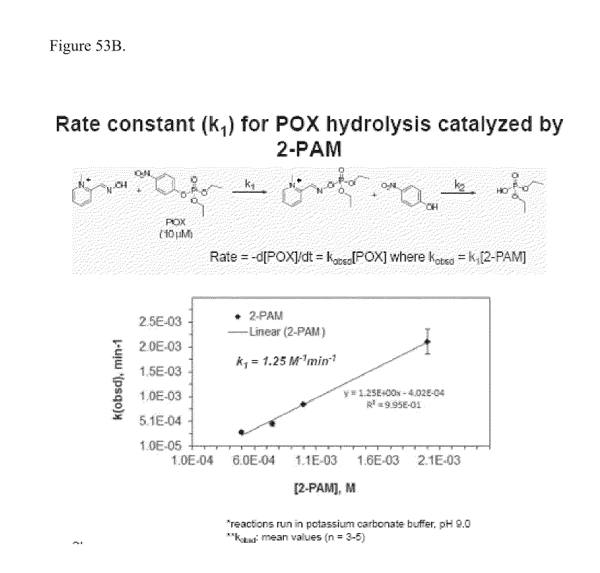

FIGS. 53A and 53B show additional data related to hydrolysis of paraoxon catalyzed by 2-PAM.

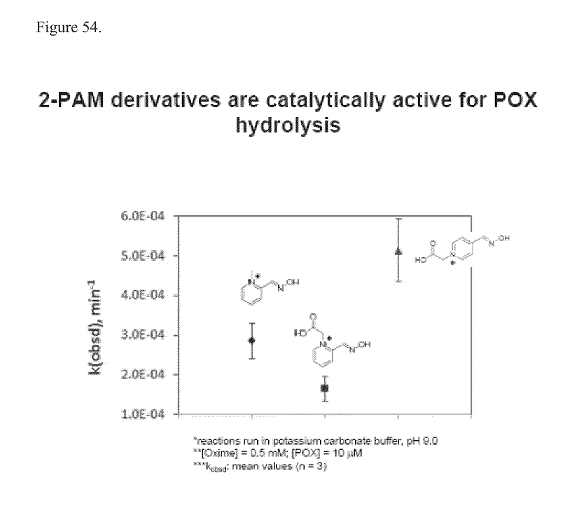

FIG. 54 shows that 2-PAM derivatives are catalytically active for PDX hydrolysis.

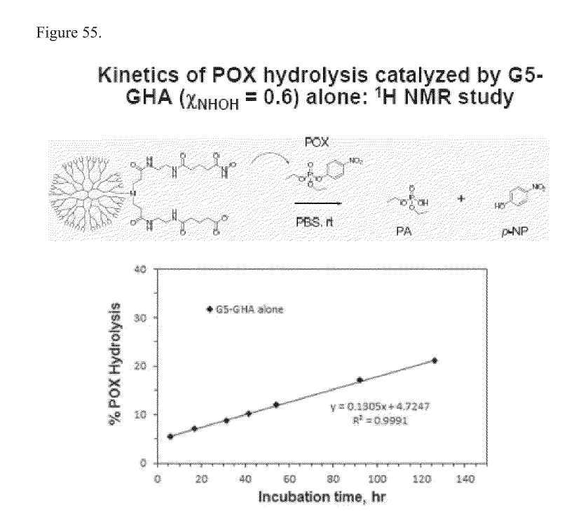

FIG. 55 shows that kinetics of PDX hydrolysis is catalyzed by G5-GHA (χ_{NHOH}=0.6) alone.

PAM Linkers

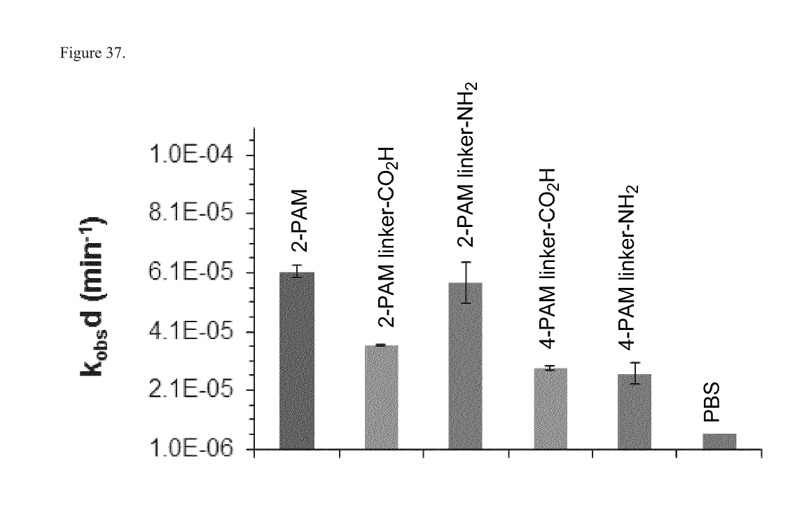

FIG. 37 shows the chemical scavenging activity at pH 7.4 displayed by four PAM linker molecules, each terminated with NH₂ or CO₂H as the chemical handle for the covalent attachment to the dendrimer. Of those, 2-PAM linker-NH₂ is almost as active as 2-PAM, and the remaining molecules showed the scavenging activity approximately 2-fold lower than 2-PAM. This result suggests that the installation of the linker for 2-PAM or 4-PAM at the nitrogen position retains the activity.

NMR Spectroscopy

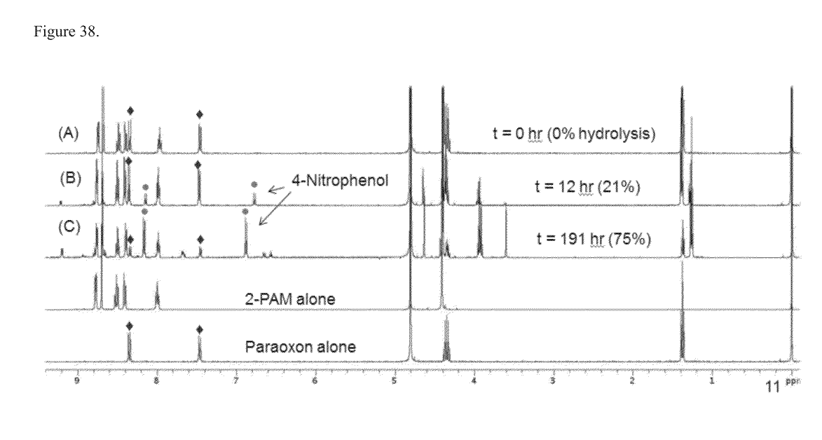

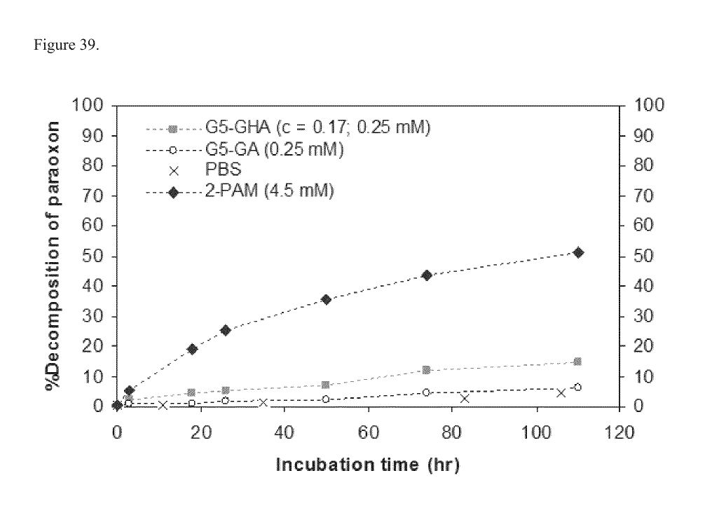

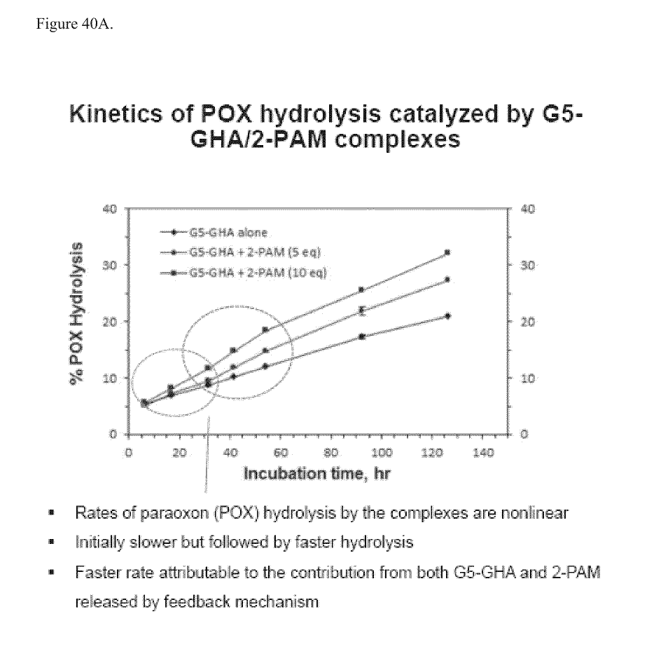

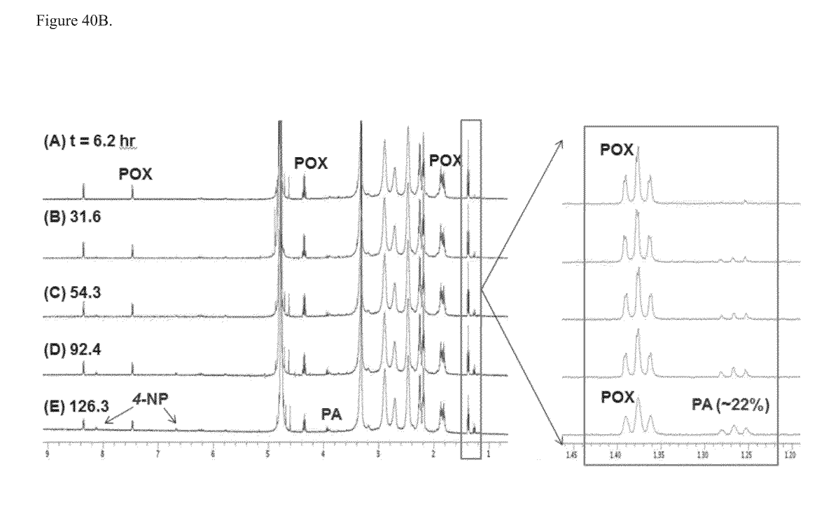

Nuclear magnetic resonance (NMR) spectroscopy was employed as the complimentary method to evaluate the chemical scavenging activity for the OP destruction. This NMR method requires larger amounts of each reactant to be studied and is more suitable for studying the conditions that require higher concentrations of PDX. The ¹H NMR spectra shown in FIG. 38 illustrates the progress of paraoxon (PDX; 0.5 mM) hydrolysis as a function of time. This reaction was catalyzed by 2-PAM at the equimolar concentration (0.5 mM) in deuterated PBS (pH 7.4) at rt. The aromatic signals for free 4-nitrophenol (4-NP) grew over time as the result of PDX hydrolysis. Integration of the area for each 4-NP signal was performed for each time point and it was compared to that for intact PDX signal determined in the same manner. Their integration ratio enabled to determine the amount of PDX hydrolyzed: t=12 hr (21%), 191 hr (75%). ¹H NMR spectroscopy was performed for monitoring the hydrolysis of paraoxon (PDX; 0.5 mM) catalyzed by G5-GHA (n=66; 0.05 mM) at pH 7.4. The PDX scavenging activity of the dendrimer hydroxamate was observed even at this lower dendrimer concentration ([G5-GHA]/[PDX]=0.1), and approximately 22% of PDX was destroyed after incubation for 126 hr at rt. FIG. 39 summarizes the similar results obtained in the condition that used the identical concentration of PDX (4.5 mM). In summary, the NMR method was employed, and the PDX scavenging activity evaluated by oxime drugs and the dendrimer drug conjugates. FIG. 40B shows ¹H NMR spectroscopy for determining the kinetics of paraoxon (PDX; 0.5 mM) hydrolysis catalyzed by G5-GHA (n=66; 0.05 mM) in deuterated PBS (pH 7.4). The PDX hydrolysis was studied at rt. Note that 4-NP (4-nitrophenol), and PA (diethylphosphoric acid) are the two degradation products of PDX.

Feedback-Regulated Drug Release

After demonstration of the OP scavenging activity by the hydroxamate-conjugated dendrimers (G5-GHA) as its built-in therapeutic activity, the NMR study was continued to study the feedback-regulated release mechanism as proposed. The G5-GHA (χ=0.6; 0.0486 mM) was selected, and prepared two dendrimer-drug complexes, each in complex with 2-PAM molecules but at a different drug to dendrimer ratio ([2-PAM]/[G5-GHA]=5 or 10). The rate of PDX (0.476 mM) hydrolysis was determined for each G5-GHA/2-PAM complex or G5-GHA itself (pH 7.4, rt) by ¹H NMR spectroscopy. FIG. 40 summarizes the relative rates of hydrolysis by plotting the percent amount of PDX hydrolyzed as a function of incubation time. The rate for the G5-GHA alone shows a single linear slope, but the rate for each complex shows nonlinear slopes (see the region marked by two circles). In the latter case, the slope for the hydrolysis is lower initially, but becomes higher (faster) later. It is believed that this nonlinear kinetics of PDX hydrolysis results from the combination of two catalytic components. First, its initial rate is determined primarily by G5-GHA itself because most of 2-PAM molecules are still bound in the dendrimer and thus not able to make contribution for the scavenging activity. Second, as 2-PAM molecules are released in response to individual OP scavenging reactions occurring on the dendrimer periphery, the rate is accordingly affected and enhanced due to the catalytic contribution made by 2-PAM molecules released. This study illustrates that the drug release is regulated by the OP-responsive feedback mechanism.

Mass Spectrometry.

Kinetics of PDX hydrolysis was studied in guinea pig plasma by using LCMS/MS. A plasma solution from guinea pig was prepared by spinning at 10,000 rpm for 10 min of its blood which was stored in a heparin coated tube.

In a control run, 25 μL of the plasma solution was placed in an Eppendorf vial, and diluted with 20 μL of PBS prior to the addition of 5 μL PDX in MeCN (300 μM). The mixture was vortexed, and immediately, 10 μL of aliquot was taken into a separate vial containing 190 μL of cold methanol (t₁). This methanol-treated solution was immediately frozen with liquid nitrogen and stored in the freezer (−80° C.) until it was further processed along with other samples to be generated later. The 40 μL solution remained in the vial continued to be kept in an incubator (37±2° C.) for a day during which 10 μL aliquot was taken out each at a specific time point and treated similarly with methanol (t₂=4 hr, t₃=6 hr, t₄=24 hr). Each of the methanol-treated samples was processed further by spinning down at 10,000 rpm for 10 min in a cold centrifuge. The supernatant was separated and stored immediately in a freezer (−80° C.) until it was analyzed by mass spectrometry. This plasma control experiment was repeated independently more than 5 times.

In a representative test run, 25 μL of the plasma solution was diluted with 15 μL of PBS and 5 μL of 2-PAM (15 mM) and G5-(GHA)ₙ₌₆₆ (1.2 mM). Each solution was mixed with 5 μL PDX in MeCN (300 μM), and incubated at 37° C. while a series of aliquots were taken out at the time points indicated in the control run. Each aliquot was treated with methanol and processed in the same manner as described above. This test experiment was performed in at least triplicate, each for 2-PAM and G5-(GHA)ₙ₌₆₆. On the day of the LCMS/MS analysis, 50 μL of the saved supernatant was diluted with 200 μL of an LCMS/MS eluent (9:1 ammonium formate (10 mM)/acetonitrile) and injected for the analysis.

LCMS/MS analysis was performed by using a Waters Acquity UPLC system equipped with the Waters TQ detector mass spectrometer. The method for the LC system includes: i) ODS column (XBridge BEH C18 2.5 um; 2.1×50 mm, Waters); ii) gradient elution starting with 90% aqueous ammonium formate (10 mM; A)/10% aqueous acetonitrile (B) and ends with 50/50 (A/B) in 5 min; iii) flow rate=0.5 ml/min; iv) column temperature=40° C. Detection of 4-NP is based on single reaction monitoring (SRM) parameters (negative ionization mode; source temperature=150° C.; desolvation temperature=400° C.; cone voltage=38 V; collision energy=16 eV). 4-NP was detected and quantified by focusing on a molecular species at tᵣ=2.6 min in the LC trace that has a parent mass (m/z) of 137.95.

Calibration curves for 4-NP were generated in triplicate in the range of 0.5 nM to 100 nM using an LCMS/MS eluent as the solvent. Another set of calibration curves were also generated in triplicate for 4-NP spiked in guinea pig plasma and processed in the same manner. Limit of detection (LOD) determined for 4-NP was lower than the nanomolar concentration (0.125-0.25 nM).

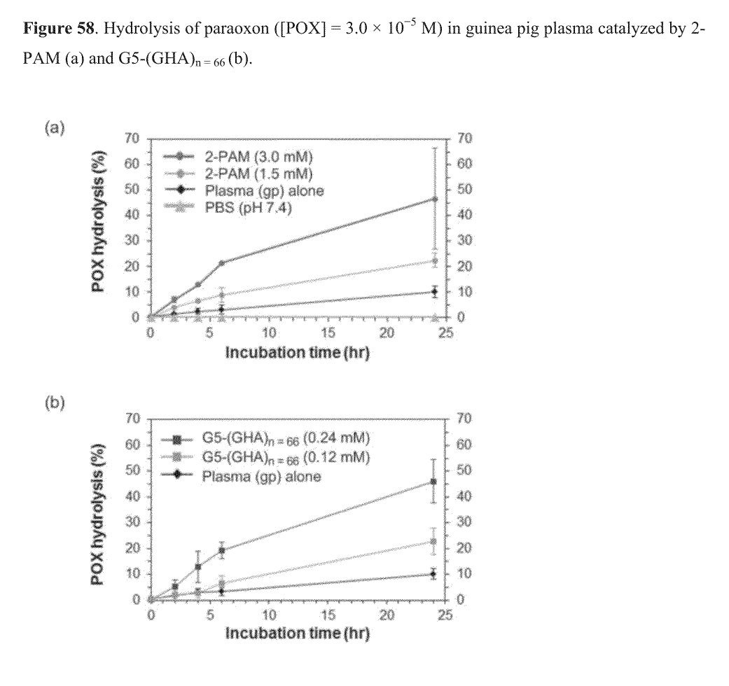

FIG. 58 summarizes the hydrolysis (%) of paraoxon catalyzed by 2-PAM or G5-(GHA)ₙ₌₆₆, each tested in guinea pig plasma.

### Example IV

This example describes the materials and methods for Example V.

### 1) Materials and Characterization

Deuterium oxide (99.9 atom % D, containing 0.05 wt % 3-(trimethylsilyl)propionic-2,2,3,3-d4 acid, sodium salt), pralidoxime chloride (pyridine-2-aldoxime methochloride, 99%) and obidoxime chloride (≧95%) were all purchased from Sigma-Aldrich, and used as received. Phosphate buffered saline (PBS) powder was purchased from Sigma-Aldrich, and dissolved in deuterium oxide to prepare 1×PBS (0.01 M phosphate, NaCl 0.138 M, KCl 0.0027 M, pH 7.4) following the instruction on the powder pouch.

Generation 5 (G5) poly(amidoamine) (PAMAM) dendrimer was purchased as a 17.5% (wt/wt) methanol solution (Dendritech, Inc., Midland, Mich.). The dendrimer was purified prior to use. Typically the methanol solution (15 mL) was concentrated by a rotary evaporator under a vacuum, and the residue was diluted with deionized water (20 mL). This solution was loaded into a membrane dialysis bag (MWCO 10 kDa), and dialyzed against deionized water (4×4 L) over 3 days. The aqueous solution was collected and freeze dried to afford G5 PAMAM dendrimer as colorless glassy solid (˜60-70% wt recovery). The average number of primary amines per dendrimer molecule was determined by potentiometric titration as described elsewhere (see, e.g., Majoros, I. J.; Thomas, T. P.; Mehta, C. B.; Baker, J. R. J. Med. Chem. 2005, 48, 5892-5899; incorporated herein by reference in its entirety). The titration was carried out manually using a Mettler Toledo MP230 pH meter and an InLab® Micro electrode at room temperature, 23±1° C. The average number (n) of primary amines per dendrimer was determined by back-titration (n=114).

Molecular weight of PAMAM G5 dendrimer was measured by matrix assisted laser desorption ionization time of flight (MALDI TOF) with a Waters TOfsPec-2E spectrometer. The MALDI spectra were acquired using a matrix solution of 2,5-dihydroxybenzoic acid (10 mg/ml in 50% aqueous acetonitrile) in a linear mode with a high mass detector, and data was processed using Mass Lynx 3.5 software. Molecular weight (MW) of the purified PAMAM dendrimer was determined to be 27600 gmol-1, a mean value calculated from three independent measurements.

Gel permeation chromatography (GPC) was used to measure polydispersity index (PDI) of PAMAM G5 dendrimer (see, e.g., Thomas, T. P.; J. Bioorg. Med. Chem. Lett. 2010, 20, 5191-5194; incorporated herein by reference in its entirety). The GPC experiment was performed on an Alliance Waters 2695 separation module equipped with a 2487 dual wavelength UV absorbance detector (Waters Corporation), a Wyatt HELEOS Multi Angle Laser Light Scattering (MALLS) detector, and an Optilab rEX differential refractometer (Wyatt Technology Corporation). Columns employed were TosoHaas TSK-Gel Guard PHW 06762 (75 mm×7.5 mm, 12 mm), G 2000 PW 05761 (300 mm×7.5 mm, 10 mm), G 3000 PW 05762 (300 mm×7.5 mm, 10 mm), and G 4000 PW (300 mm×7.5 mm, 17 mm). Column temperature was maintained at 25±0.1° C. with a Waters temperature control module. The isocratic mobile phase was 0.1 M citric acid and 0.025 wt % sodium azide, pH 2.74, at a flow rate of 1 mL/min. The sample concentration was 10 mg/5 mL. The weight average molecular weight (Mw) was determined by GPC (w M=26550 gmol-1), and the number average molecular weight (n M=26270 gmol-1) was calculated with Astra 5.3.14 software (Wyatt Technology Corporation) based on the molecular weight distribution. A polydispersity index (PDI=Mₙ/M_{w}) value determined for the purified G5 PAMAM dendrimer is 1.010.

GPC analysis for G5PAMAM dendrimers was carried out with an 100% mass recovery assumption. In a separate experiment to ascertain a dn/dc value, a known amount of G5 PAMAM dednrimer each from 7 different batches was analyzed using the GPC setup. To calculate dn/dc values, an inbuilt template in the ASTRA 5.3.14 was utilized that uses the instrument calibration constant and the amount of sample injected (concentration). The average dn/dc value obtained was 0.337+0.024. dn/dc values for various generation of PAMAM dendrimer were also calculated, and the dn/dc values were in the similar range.

### 2) NMR Experiments

One dimensional (1D) titration, and two dimensional (2D) NMR experiments (COSY, NOESY) were performed at 499.9 MHz (11.7 Tesla) for 1H nucleus using a Varian NMR spectrometer. The spectrometer was equipped with Performa I, Z-axis pulsed field gradient module and automatic gradient shimming module. Diffusion ordered spectroscopy (DOSY) NMR experiments were carried out using another Varian NMR spectrometer at 499.9 MHz (11.7 Tesla) for 1H nucleus. This spectrometer was equipped with a pulsed field gradient (62 G/cm) amplifier, and a dual channel protune module. Chemical shifts (δ) in each 1H NMR spectrum were measured in ppm, and referenced to internal 2,2-dimethyl-2-silapentane-5-sulfonate sodium salt (DSS; δ=0.00). All 1D and 2D experiments were performed at 297.3 K (±0.2) using standard pulse sequences unless noted otherwise in the specific NMR experiments described below.

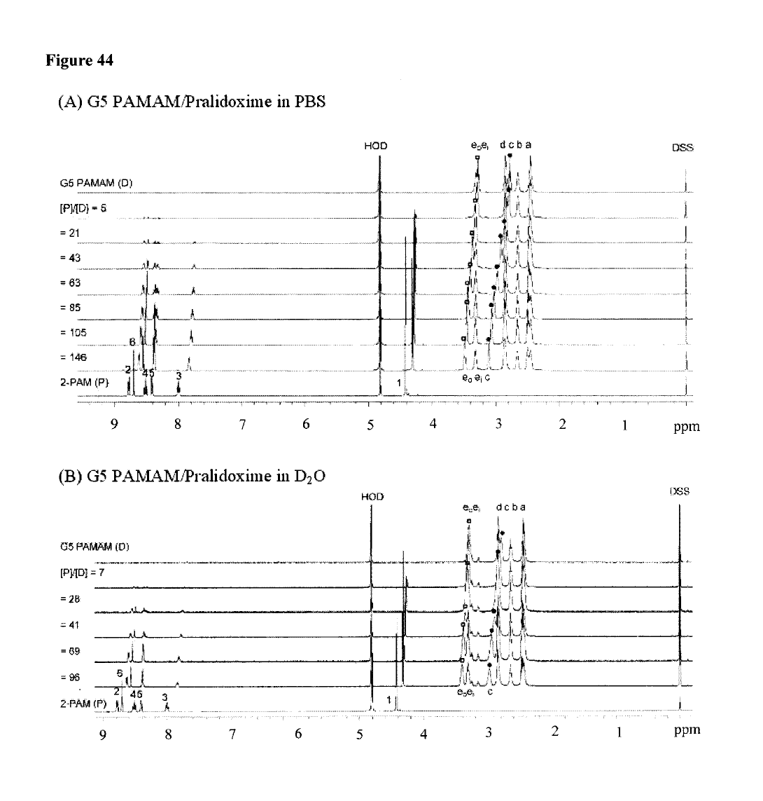

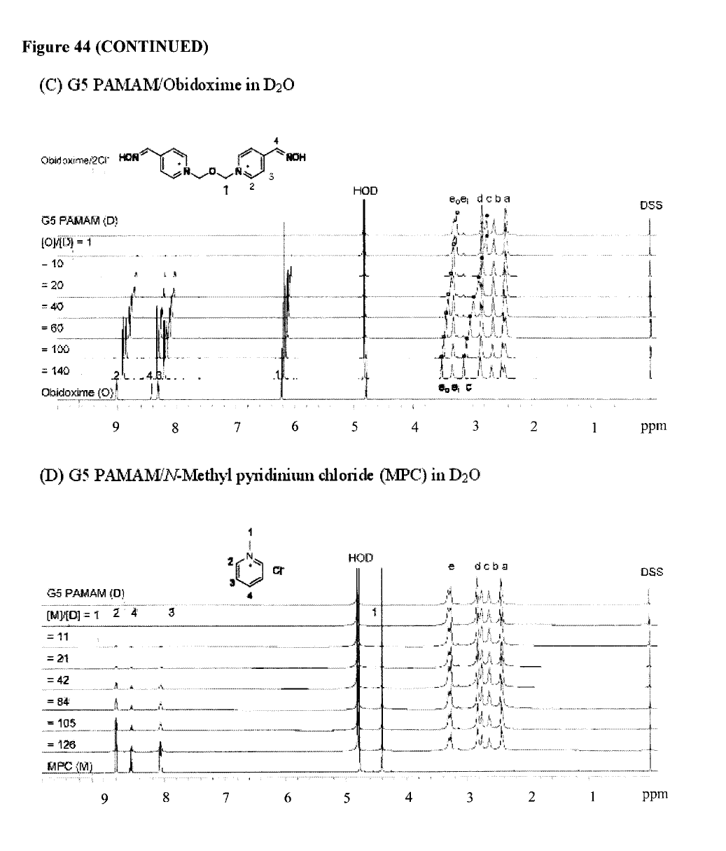

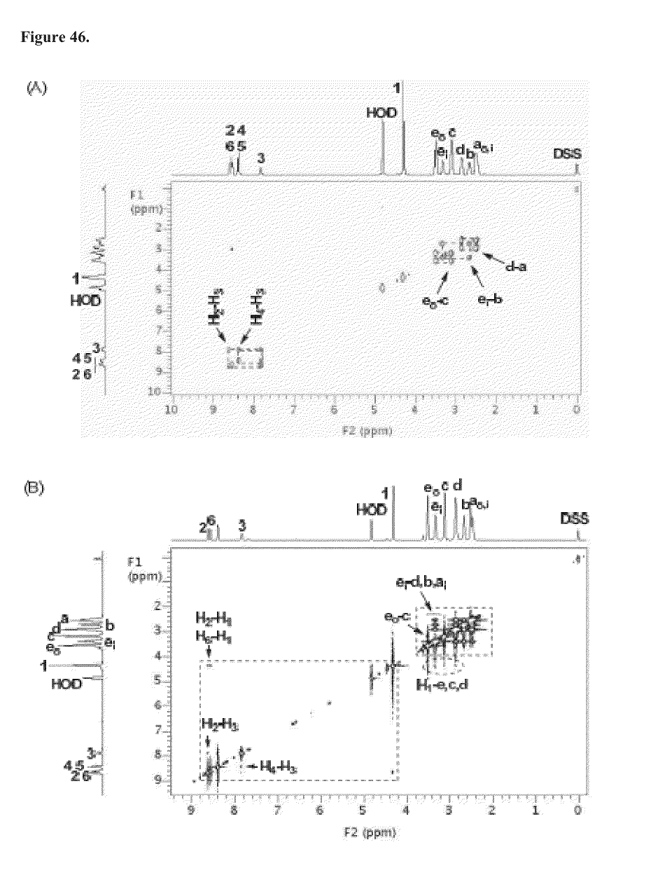

NMR titration experiments were carried out by acquiring 1H NMR spectra on samples of G5 PAMAM dendrimer in deuterium oxide ([D]=6.23×10-4M) with the addition of increasing amounts of pralidoxime (2-PAM) or obidoxime. The experiments were performed in duplicate for each drug. Fractions for bound drugs at equilibrium were determined by the equation under a fast exchange condition: δobsd=(Frfree×δfree+Frbound×δbound) where (Frfree+Frbound)=1. Protons of the pralidoxime molecule applied for thisequation comprise of H1 (N—CH3), H2, H3, and H6. Those for the obidoxime molecule include H2, H3, and H4 (see, FIG. 44C). The chemical shift value at the free state (δfree) refers to that of a free drug molecule (alone) in D2O or deuterated PBS solution. The chemical shift value at the bound state (δbound) is determined from that of a drug molecule bound to the dendrimer where [drug]/[D] is 1 or less than 5. Typically, the fraction of bound drug molecules was determined as average of fractional values obtained from multiple protons. Fractions of bound drug molecules and the occupied binding sites shown in FIG. 46A, B are given as mean of the averages from the three independent measurements for each complex. The error bars in the Figure represent standard deviations calculated from the mean value. The uncertainty value associated with the measurement of chemical shift (δ) is ±0.002 ppm.

2D 1H-1H COSY experiments for the dendrimer-oxime drug complexes in solution were performed each at 7.9 μs of 1H pulse width, 1 s of relaxation delay, and 0.15 s of acquisition time. Number of scans per t1 increment, and number of t1 increment are 2, and 128, respectively. 2D 1H-1H NOESY experiments for the dendrimer-oxime drug complexes in solution were performed with a nOe mixing time of 200 ms and 10.44 μs of 1H pulse width. The data acquisition was carried out at relation delay of 2 s, and acquisition time of 0.2 s. Eight transients were averaged per t1 increment, and number of t1 increment was 128. Each of the 2D spectral data was processed using Varian's Vnmr J software.

The DOSY NMR study was performed for each dendrimer-pralidoxime complex prepared in deuterium oxide solution ([D]=6.04×10-5M; [2-PAM]/[D]=0, 7, 21, 48, 69, 96, 123.5). The experiments were carried out using the DOSY gradient compensated stimulated echo with spin lock and convection compensation (DgcsteSL_cc), which is an enhancement of the classical pulsed gradient spin-echo (PGSE) pulse sequence (see, e.g., Alvarado, E.; University Of Michigan: Ann Arbor, 2010; incorporated herein by reference in its entirety). Key DOSY parameters include 15 increments in the gradient strength, 2.0 ms of diffusion gradient length, and 200 ms of diffusion delay. Diffusion gradient level was set up from 0 to 2048, a maximum value allowed by the gradient amplifier. These numbers are in an arbitrary scale without units provided by a digital-to-analog converter (DAC), and the instrument was calibrated with D2O at 290 K to gauss/cm (0.00961 gauss/(cm×DAC)). DOSY spectral data were processed using Varian's VnmrJ software. Diffusion coefficient (D, m2s-1) for a given dendrimer-drug complex was determined by using a default method 3 in the software which is based on fitting of the integration of the dendrimer peak to the Stejskal-Tanner function (see, e.g., Pelta, M. D.; Magn. Reson. Chem. 1998, 36, 706-714; incorporated by reference in its entirety)

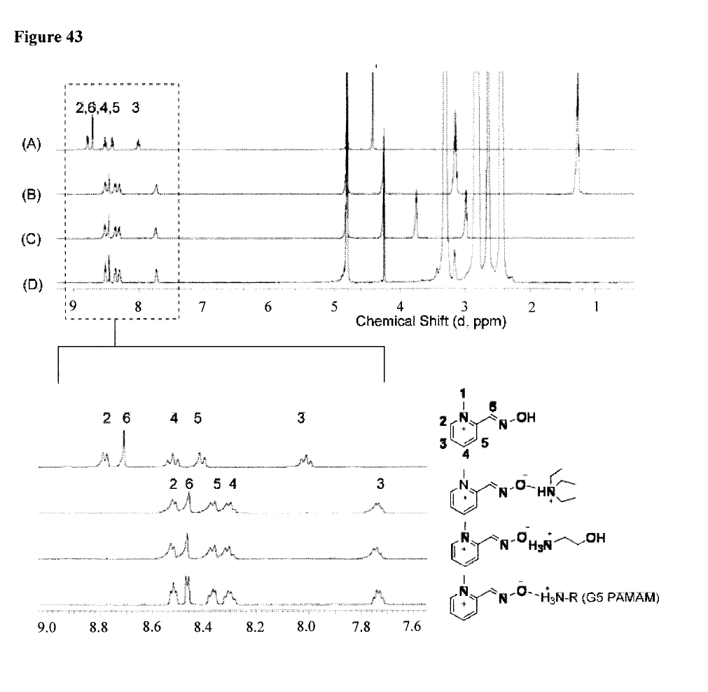

ln(I/I0)=−γ2δ2G2(Δ−δ/3)D

where:
I=intensity or integral of the peak at a given G
I0=intensity or integral of the peak at G=0
γ=magnetogyric constant of the nucleus (for 1H, γ=2.675×108 T-1s-1)
δ=diffusion gradient length
Δ=diffusion delay
G=gradient field strength
D=diffusion coefficient
   Diffusion coefficient determined for each complex in FIG. 43B refers to a mean value obtained from at least three independent sets of measurements, and the error represents the standard deviation from the mean value.

### Example V

Pralidoxime (2-PAM) and obidoxime belong to a class of oxime antidotes developed for the treatment of organophosphate poisoning (see, e.g., Edery, H.; Science 1958, 128, 1137-1138; Cohen, S.; J. Med. Chem. 1971, 14, 621-626; each herein incorporated by reference in its entirety). Both drugs have short durations of action that could, in principle, be extended by complexing the drugs to nanocarriers for increasing their circulation half-lives. In a first study 1H NMR titration experiments were performed to locate structural determinants for the complex formation between a generation 5 (G5) PAMAM dendrimer and 2-PAM (FIG. 1). Upon the addition of 2-PAM, only a few subsets of the dendrimer protons that belong to terminal branches (c, eo, ao) apparently shifted downfield as a function of the [2-PAM]/[D] ratio, while other inner protons remained almost unchanged. The relative magnitudes of such changes (c, eₒ, aₒ) suggest that binding of the guest molecules selectively occurs at the terminal branches of the dendrimer. On the guest side, the proton signal associated with 2-PAM shifted upfield as the ratio decreased.

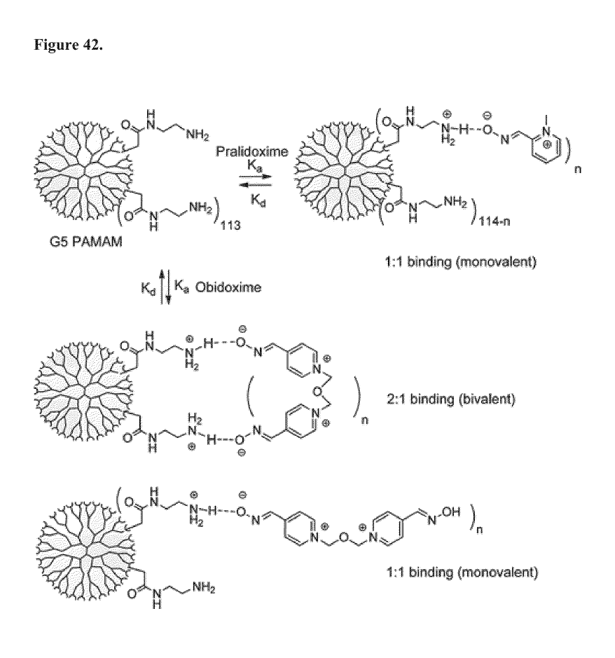

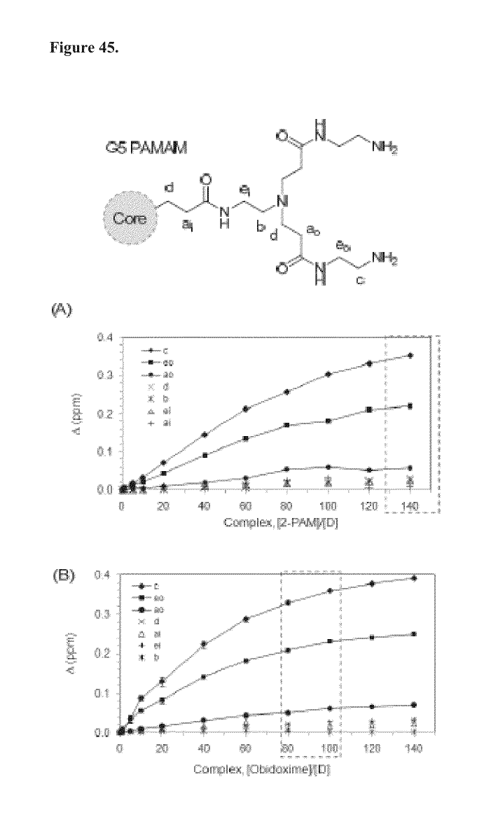

Given the pKaof 2-PAM (8.1) (see, e.g., Karljikovic-Rajic, K.; J. Pharm. Biomed. Anal. 1990, 8, 705-709; %%), lower than the pKa of a terminal primary amine (9.0-10.77) (see, e.g., Cakara, D.; Macromolecules 2003, 36, 4201-4207; Diallo, M. S.; Langmuir 2004, 20, 2640-2651; each herein incorporated by reference in its entirety), it was hypothesized that electrostatic interaction is the driving force for the drug complexation (FIG. 42). This hypothesis was verified by measuring the 1HNMR spectra of 2-PAM mixed with an equimolar amount of triethylamine (pKa=10.78) and of ethanolamine (pKa=9.50). Here, the Δ values for 2-PAM observed in a bound state ([2-PAM]/[D]=10) are correlated with those from each of the mixtures (FIG. 43). Furthermore, the 1H NMR titration experiments performed with N-methylpyridinium chloride (MPC), a molecule that lacks such an aldoxime moiety, under otherwise an identical condition led to no evidence for the complexation (FIG. 44). In contrast, the titration experiments performed with obidoxime resulted in the changes in chemical shifts that are consistent with those seen with 2-PAM (FIGS. 44 and 45). Thus, obidoxime, like 2-PAM, binds to the dendrimer through the electrostatic interactions. While the above experiments were carried out in a nonionic solution (D2O), the same experiments performed for 2-PAM in a high ionic strength solution (PBS pH 7.4, I=0.15) led to almost identical complexation trends (FIGS. 44 and 45).

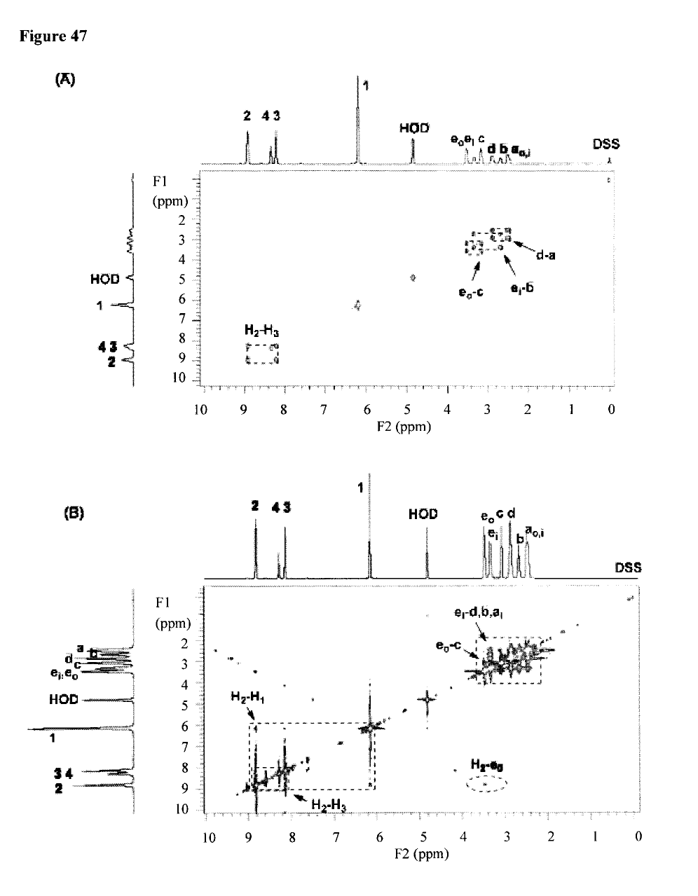

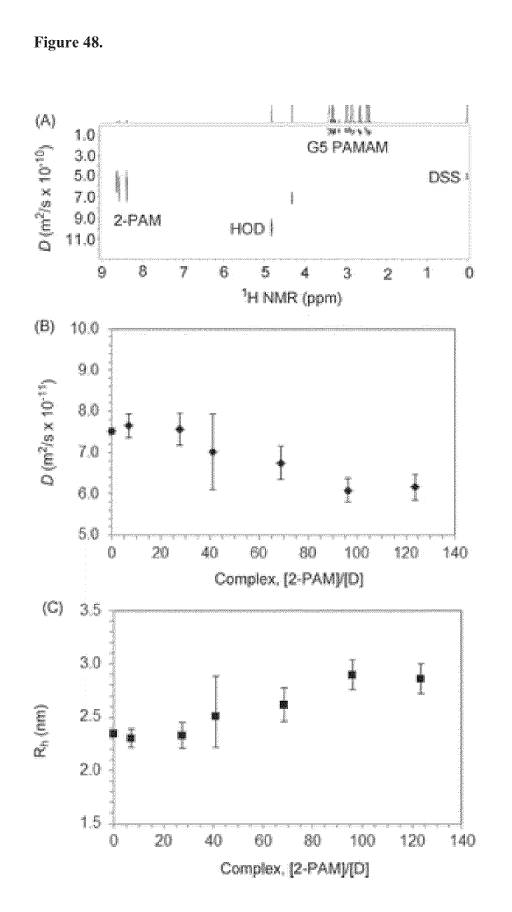

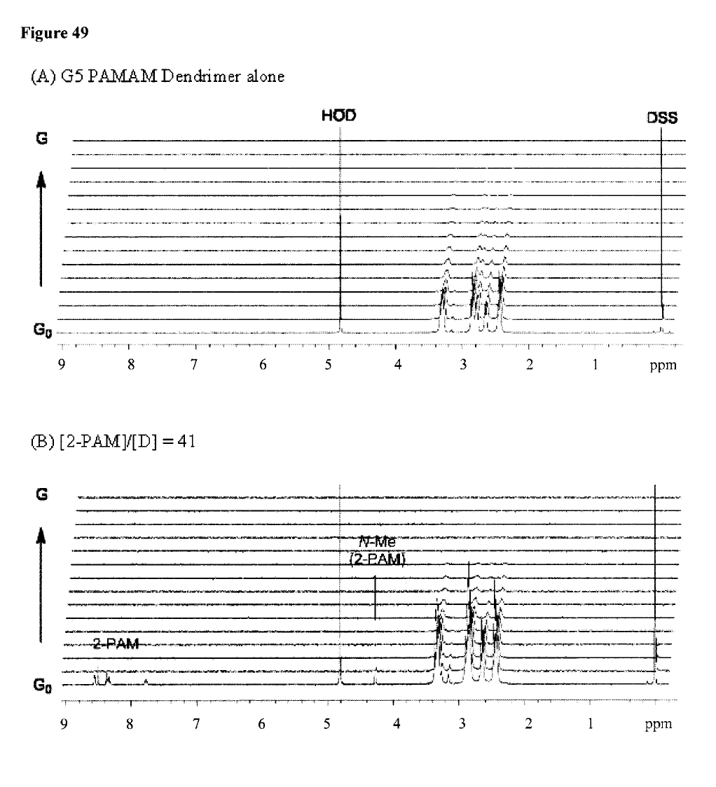

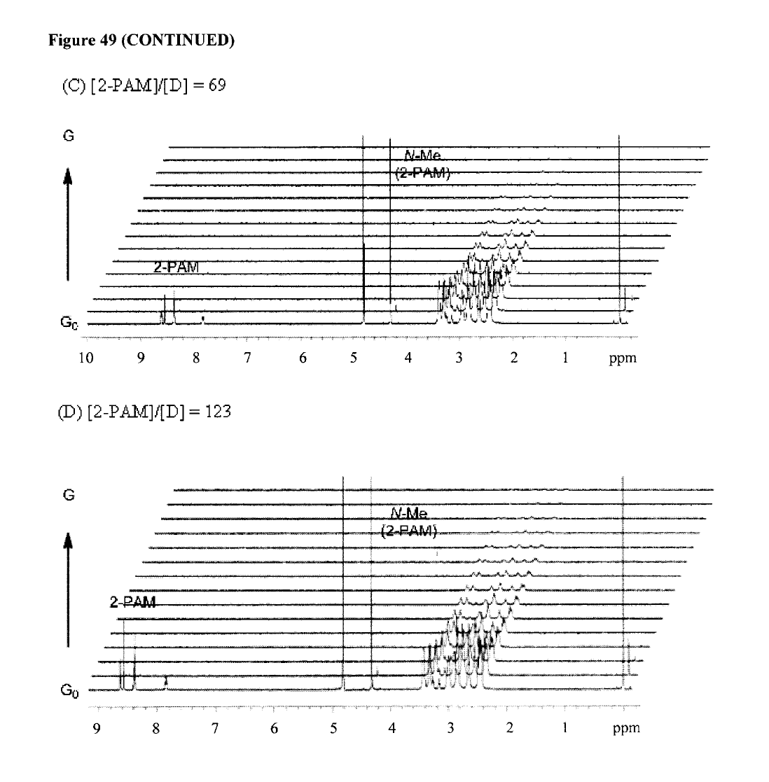

The binding models proposed in FIG. 42 were next explored by using other NMR techniques. First, 2D ¹H-¹H COSY and NOESY NMR experiments were performed for the dendrimer complexes with the oxime drugs (FIGS. 46 and 47). Notably, certain cross-peaks observed in the NOESY spectra are attributable to through-space intermolecular correlation, an evidence for spatial proximity (d≦5 Å) between the drug molecules and dendrimer branches (H₁-eₒ,c,d for 2-PAM; H₂-eₒ for obidoxime). Second, hydrodynamic properties of dendrimer/2-PAM complexes by ¹H diffusion-ordered spectroscopy (DOSY) was studied (FIG. 48). Diffusion coefficients (D, m² s⁻¹) determined for the complexes by fitting the peak-integration decay curves of the DOSY spectra (FIG. 49) decrease relative to the dendrimer alone (D=(7.49±0.30)×10⁻¹¹ m² s⁻¹) and in response to the ratio [2-PAM]/[D]. The diffusion coefficients allowed to calculate hydrodynamic radii (Rₕ) for the complexes according to the Einstein-Stokes equation (eq 1, where η=viscosity of D₂O=1.24×10⁻³ kg m⁻¹ s⁻¹ and other parameters defined in the literature) (see, e.g., Gomez, M. V.; J. Am. Chem. Soc. 2009, 131, 341-350; Pavan, G. M.; Chem.-Eur. J. 2010, 16, 7781-7795; each herein incorporated by reference in its entirety).

D=κ_{B}T(1−κφ)/6πηRₕ (1)

The dendrimer complexes display greater hydrodynamic radii than the free dendrimer (Rₕ=2.35±0.09 nm) in a drug concentration-dependent manner, which strongly supports the formation of specific complexes.

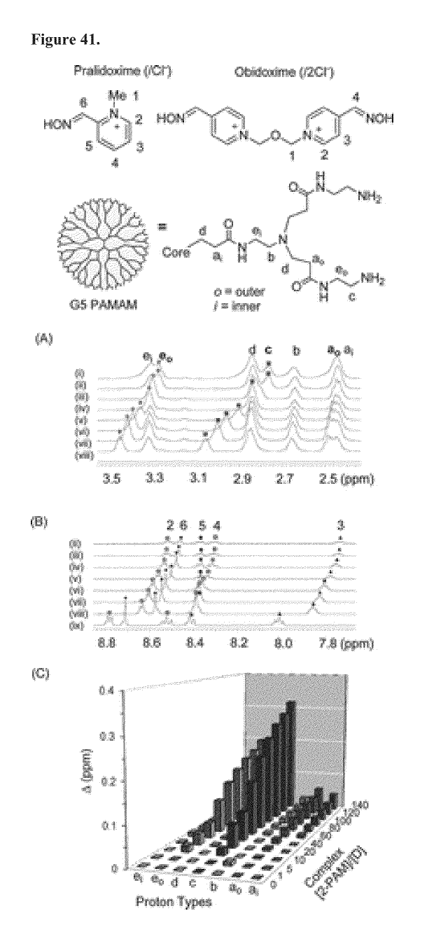

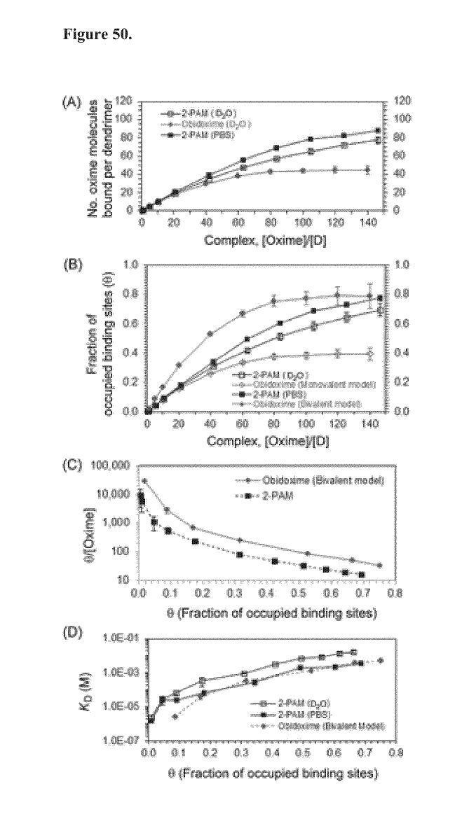

In efforts to quantitatively understand individual binding events in ¹H NMR titration experiments (FIGS. 41 and 44), the fractions of drugs bound at steady state were calculated and, complementarily, those of occupied binding sites. According to the NMR responses, the present dendrimer-drug complexation belongs to a system undergoing fast on/off exchange and could be analyzed by eq 2, which determines fractions for the drugs bound (FIG. 50) (see, e.g., Fielding, L. Prog. Nucl. Magn. Reson. Spectrosc. 2007, 51, 219-242; %%). Unlike 2-PAM, obidoxime has a C₂ symmetry with two identical aldoxime moieties, and thus two modes of association (monovalent, bivalent) were considered separately for analysis (FIG. 42).

δ_{obsd}=(Fr_{free}×δ_{free}+Fr_{bound}×δ_{bound}) (2)

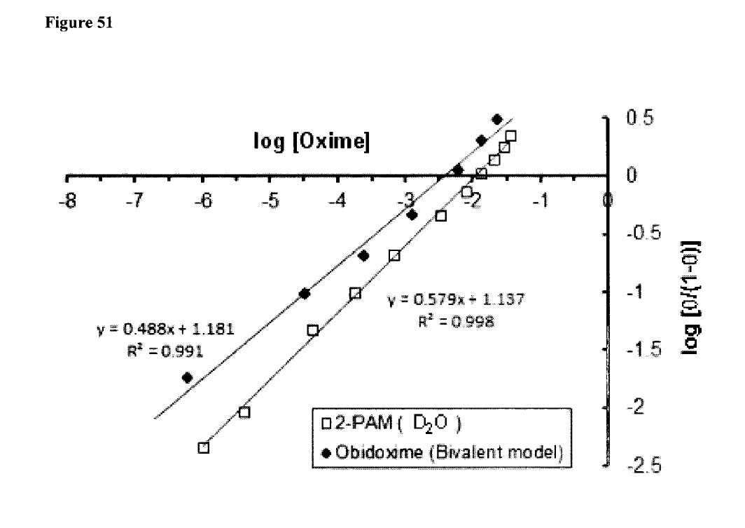

FIG. 50A illustrates that the number of bound oxime molecules increases as a function of the ratio [oxime]/[D]. Binding of 2-PAM in D₂O appears to be saturated at the level of 78 drugs bound per dendrimer ([2-PAM]/[D]=145). The ionic strength of the medium affects the binding level such that 2-PAM molecules bound more in PBS than D₂O by up to 10 drug molecules per dendrimer. Such difference is attributable to, for example, structural and conformational flexibility of the dendrimer which is influenced by external factors such as solvent, pH, and ionic strength (see, e.g., Tomalia, D. A.; Angew. Chem., Int. Ed. 1990, 29, 138-175; Maiti, P. K.; Macromolecules 2005, 38, 979-991; Liu, Y.; J. Am. Chem. Soc. 2009, 131, 2798-2799; Porcar, L.; J. Phys. Chem. B 2010, 114, 1751-1756; each herein incorporated by reference in its entirety). Qualitatively, it is plausible that the presence of counterions associated with the dendrimer scaffold may open up dendritic branches by interrupting their intramolecular interactions and as a consequence relieve the degree of unfavorable steric congestion arising from drug binding. Remarkably, obidoxime shows a saturation behavior with its maximum (≈40 bound per dendrimer) reached at the lower ratio ([obidoxime]/[D]≈80). Such a drug saturation curve corresponds with the dendrimer response curve (FIG. 45). The changes (Δ) for the terminal branches reach the maximal level at a similar ratio ([obidoxime]/[D]≈90 where ˜40 obidoxime molecules bound). In addition, its level is very comparable to that observed in experiments with the other drug ([2-PAM]/[D]≈145 where ˜80 of 2-PAM molecules are bound). These findings are supportive of functional bivalency of obidoxime and consistent with the fractional analysis of occupied binding sites (θ) for obidoxime calculated on the basis of its bivalent model (FIG. 50B). Scatchard analysis performed for each drug shows nonlinear decay, and thus, instead of calculating average affinity, the affinity distribution was estimated as a function of θ (FIGS. 50C and 50D). Generally, affinities are greater for obidoxime than pralidoxime in D₂O, suggesting the difference in their modes of binding (bivalent vs monovalent). In addition, the affinities are higher (K_{D}˜10⁻⁶ M) at lower binding fractions (θ<0.1) and decrease as more sites are occupied, indicative of repulsive interactions between successive binding events. The Hill coefficient (n) determined for each drug provides a quantitative index for such negative cooperativity with values of 0.58 (2-PAM) and 0.49 (obidoxime) (FIG. 51). It is believed that steric congestion plays a dominant role for this effect as suggested in a broad range of antibody-antigen recognition processes and specifically in the reactions catalyzed by metallodendrimers (see, e.g., Kleij, A. W.; Angew. Chem., Int. Ed. 2000, 39, 176-178; Edberg, S. C.; J. Immunochemistry 1972, 9, 273-288; Goldstein, B. Biophys. Chem. 1975, 3, 363-367; each herein incorporated by reference in its entirety). However, it is in contrast to the proximity effects reported for other dendrimer-based catalytic reactions (see, e.g., Breinbauer, R.; Angew. Chem., Int. Ed. 2000, 39, 3604-3607; Francavilla, C.; J. Am. Chem. Soc. 2000, 123, 57-67; each herein incorporated by reference in its entirety), suggesting that substrate binding in the catalytic dendrimer might be less sensitive to steric effects due to its rapid turnover.

### Example VI

This example describes a strategy for co-presentation of oxime antidotes and auxiliary metal chelators.

The present invention further provides another class of polyamidoamine (PAMAM) dendrimers, each conjugated with both oxime-based therapeutic molecules and metal chelators (FIG. 56). The therapeutic benefit for attaching such auxiliary groups is illustrated in the proposed mechanism of OP (PDX) hydrolysis where the auxiliary group plays a significant role by facilitating the catalytic reaction mediated by the oxime or hydroxamate of the attached drug molecule. Such auxiliary groups for metal chelation are based on the amine, imidazole, pyridine, and carboxylate group such as Tren, PDA, and PCA, but not limited here. Metal ions to be chelated include zinc, copper and other physiologic cations that are able to chelate to the P═O of the OP molecule and to make the phosphorous bond more susceptible for the hydrolytic cleavage.

Synthesis of G5-PAM Dendrimers Conjugated with Auxiliary Metal Chelators

G5-(GHA)ₙ-(Zn²⁺):

To a solution of G5-(GHA)₆₅ (10 mg) in water (1 mL) is added 1 mL of ZnCl₂ (1 mM). The mixture is shaken at room temperature for 30 min, and the unbound Zn ions in the mixture are removed by ultrafiltration (Centricon; MWCO 10,000). The residual supernatant is diluted with water to 2 mL, and the freeze-drying of the solution affords the G5-(GHA)₆₅-(Zn²) as solid.

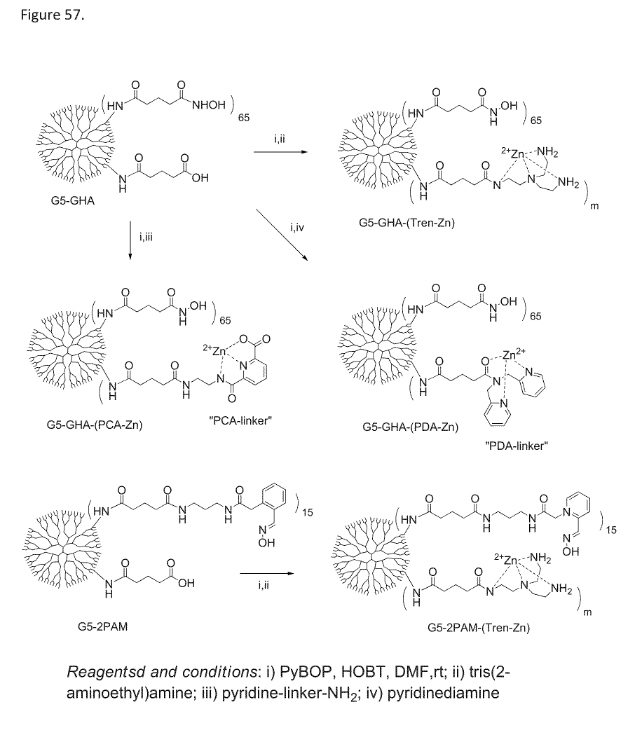

G5-(GHA)ₙ-(Tren-Zn)ₘ (FIG. 57):

To the G5-(GHA)65 dendrimer (100 mg) suspended in anhydrous DMF (50 mL) is added N-hydroxybenzotriazole (HOBt, 17 mg), 4-dimethylaminopyridine (MDAP, 29 mg), and then PyBOP (64 mg). The reaction mixture is stirred at room temperature for 24 hr until the solution becomes homogenous. Tren (18 mg) is added to the mixture at the [Tren]/[G5-GHA] ratio of 50. The final reaction mixture is stirred for additional 12 hr. The conjugation reaction is terminated by adding water (5 mL) to the mixture, and it is concentrated in vacuo, yielding dendrimer residue. To purify the denrimer product, the residue is dissolved in 10 mL of phosphate buffered saline (PBS without Ca²⁺ and Mg²⁺, pH 7.4). The solution is loaded into a membrane dialysis bag (Spectrum® Labs, Inc.; MWCO 10 kDa), and dialyzed against PBS (2×2 L), and deionized water (3×2 L) over 2 days. The aqueous solution is collected and freeze-dried to afford the G5-GHA-Tren as the colorless solid. The purity of the dendrimer is analyzed by the HPLC method (≧99%), and the number of tren attached to the peripheral branches of the dendrimer is determined on a mean basis by the integration method of ¹H NMR spectral peaks where the CH₂ of Tren-glutaric amide are used for the analysis.

The metal chelated dendrimer, G5-(GHA)₆₅-(Tren-Zn), is obtained by treatment of G5-(GHA)-(Tren) with ZnCl₂ (1 mM in water).

G5-(GHA)-(Tren) (FIG. 57):

To a mixture of G5-GA (50 mg, 1.24 μmol), NHS (33 mg, 287 μmol) and DMAP (35 mg, 287 μmol) in DMF (10 mL) was added EDC-HCl (41 mg, 214 μmol). The mixture was stirred at RT for 24 hr prior to the addition of N¹,N¹-bis(2-aminoethyl)ethane-1,2-diamine (5.5 mg, 38 μmol; [Tren]/[dendrimer]=30). The mixture was stirred for an additional 3 hr and followed by the addition of O-(TBDMS)hydroxylamine (42 mg, 286 μmol). The final mixture was stirred at RT for 12 hr prior to quenching with water (5 mL). The residue was concentrated in vacuo, yielding a pale brown residue. To purify the denrimer conjugate, the residue was dissolved in 10 mL of phosphate buffered saline (PBS without Ca²⁺ and Mg²⁺, pH 7.4) and loaded into a membrane dialysis bag (Spectrum® Labs, Inc.; MWCO 10 kDa). It was dialyzed against PBS (2×2 L), and deionized water (3×2 L) over 2 days. The aqueous solution was collected and freeze-dried to afford the G5-GHA-(Tren) as white solid (30 mg). HPLC analysis: tᵣ=7.66 min; purity ≧99%. MALDI-TOF: m/z (gmol⁻¹)=43800. UV/vis spectroscopy (PBS, pH 7.4): λₘₐₓ=294 nm.

G5-(GHA)-(PDA) (FIG. 57):

To a mixture of G5-GA (50 mg, 1.24 μmol), NHS (33 mg, 287 μmol) and DMAP (35 mg, 287 μmol) in DMF (10 mL) was added EDC (41 mg, 214 μmol). The mixture was stirred at RT for 24 hr prior to the addition of di-(2-pycolyl)amine (7.4 mg, 37 μmol; [PDA]/[dendrimer]=30). The mixture was stirred for an additional 3 hr and followed by the addition of O-(TBDMS)hydroxylamine (42 mg, 286 μmol). The final mixture was stirred at RT for 12 hr prior to quenching with water (5 mL). The residue was concentrated in vacuo, yielding a pale brown residue. To purify the denrimer conjugate, the residue was dissolved in 10 mL of phosphate buffered saline (PBS without Ca²⁺ and Mg²⁺, pH 7.4) and loaded into a membrane dialysis bag (Spectrum® Labs, Inc.; MWCO 10 kDa). It was dialyzed against PBS (2×2 L), and deionized water (3×2 L) over 2 days. The aqueous solution was collected and freeze-dried to afford the G5-GHA-(PDA) as white solid (25 mg). HPLC analysis: tᵣ=7.66 min; purity ≧99%. MALDI-TOF: m/z (gmol⁻¹)=43800. UV/vis spectroscopy (PBS, pH 7.4): λₘₐₓ=294 nm.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A method for treating a subject having organophosphate poisoning and/or preventing a subject from developing organophosphate poisoning comprising administering to the subject an effective amount of one or more dendrimers conjugated with one or more therapeutic agents, wherein said one or more therapeutic agents comprises one or more organophosphate poisoning antidote agents.

2. The method of claim 1, wherein said one or more dendrimers is selected from the group consisting of a Baker-Huang dendrimer and a PAMAM dendrimer.

3. The method of claim 1, wherein said dendrimer has a generation between 0 and 5.

4. The method of claim 1, wherein said organophosphate is one or more selected from the group consisting of parathion, paraoxon, sarin, and VX.

5. The method of claim 1, wherein said one or more organophosphate poisoning antidotes is selected from the group consisting of one or more of pralidoxime (2-PAM) (4-PAM), obidoxime, trimedoxime, hydroxamate, and asoxime (HI-6).

6. The method of claim 1, wherein said dendrimer is conjugated with said one or more organophosphate poisoning antidotes via a spacing agent.

7. The method of claim 6, wherein said spacing agent comprises a oligoethyleneglycol linear chain.

8. The method of claim 1, wherein administration of said dendrimer to said subject results in hydrolysis of organophosphate molecules.

9. The method of claim 1, wherein said administration of said dendrimer to said subject results in reactivation of inhibited acetylcholine esterase.

10. The method of claim 1, wherein said dendrimer is co-administered with one or more additional agents known to be effective in treating organophosphate poisoning, wherein said additional agents are selected from the group consisting of oxime agents, anticholinergic agents, and benzodiazepine agents.

11. A method of reactivating acetylcholine esterase inhibited by an organophosphate, comprising exposing said acetylcholine esterase to one or more dendrimers conjugated with one or more therapeutic agents, wherein said one or more therapeutic agents comprises one or more organophosphate poisoning antidote agents.

12. The method of claim 11, wherein said one or more dendrimers is selected from the group consisting of a Baker-Huang dendrimer and a PAMAM dendrimer, wherein said dendrimer has a generation between 0 and 5.

13. The method of claim 11, wherein said organophosphate is one or more selected from the group consisting of parathion, paraoxon, sarin, and VX.

14. The method of claim 11, wherein said one or more organophosphate poisoning antidotes is selected from the group consisting of one or more of pralidoxime (2-PAM) (4-PAM), obidoxime, trimedoxime, hydroxamate, and asoxime (HI-6).

15. The method of claim 11, wherein said dendrimer is conjugated with said one or more organophosphate poisoning antidotes via a spacing agent, wherein said spacing agent comprises a oligoethyleneglycol linear chain.

16. A composition comprising one or more dendrimers conjugated with one or more therapeutic agents, wherein said one or more therapeutic agents comprises one or more organophosphate poisoning antidote agents.

17. The composition of claim 16, wherein said one or more dendrimers is selected from the group consisting of a Baker-Huang dendrimer and a PAMAM dendrimer.

18. The composition of claim 16, wherein said dendrimer has a generation between 0 and 5.

19. The composition of claim 26, wherein said one or more organophosphate poisoning antidotes is selected from the group consisting of one or more of pralidoxime (2-PAM) (4-PAM), obidoxime, trimedoxime, hydroxamate, and asoxime (HI-6).

20. The composition of claim 16, wherein said dendrimer is conjugated with said one or more organophosphate poisoning antidotes via a spacing agent, wherein said spacing agent comprises a oligoethyleneglycol linear chain.
